(19) United States
(12) Patent Application Publication
(10) Pub. No.: US 2026/0327614 A1
(43) Pub. Date: Oct. 22, 2026

(54) METHOD OF MODULATING RNA SPLICING

(71) Applicant: Peking University, Beijing (CN)

(72) Inventors: Wensheng WEI, Beijing (CN); Zongyi YI, Beijing (CN); Huixian TANG, Beijing (CN)

(21) Appl. No.: 19/485,241

(22) PCT Filed: May 16, 2024
(86) PCT No.: PCT/CN2024/093632

§ 371 Date: Nov. 17, 2025

(30) Foreign Application Priority Data
May 16, 2023 (WO) PCT/CN2023/094452

(51) Int. Cl. C12N 15/113

(52) U.S. Cl. CPC C12N 15/113; C12N 2310/532; C12N 2320/33

(57) ABSTRACT

Provided are methods for inducing exon skipping in a pre-mRNA in a host cell comprising introducing a deaminase-recruiting RNA (dRNA) or a construct comprising a nucleic acid encoding the dRNA into the host cell, wherein the dRNA is a circular RNA (circRNA) or forms a circRNA within the host cell.

### CROSS-REFERENCE TO RELATED APPLICATIONS

This application is a U.S. national stage application under 35 U.S.C. § 371 of International Application No. PCT/CN2024/093632, filed internationally on May 16, 2024, which claims the benefit of priority of International Patent Application No. PCT/CN2023/094452, filed May 16, 2023, the contents of each of which are incorporated herein by reference in their entirety.

### REFERENCE TO AN ELECTRONIC SEQUENCE LISTING

The contents of the electronic sequence listing (165392001541SEQLIST.xml; Size: 48,248 bytes; and Date of Creation: May 14, 2024) is herein incorporated by reference in its entirety.

### FIELD OF INVENTION

The present disclosure relates generally to methods and compositions for modulating RNA splicing using an engineered RNA capable of recruiting an adenosine deaminase acting on RNA (ADAR) to one or more splice sites on a pre-mRNA.

### BACKGROUND

Transcription and translation constitute the process of gene expression, and splicing of precursor mRNA (pre-mRNA) is a crucial step in this process, which cuts out the introns in the pre-mRNA and connects the exons in an orderly manner for the final processing into mature mRNAs. The splicing reaction is carried out by the spliceosome, a dynamic complex of five small nuclear ribonucleoproteins (snRNPs)—U1, U2, U4, U5, and U6—and many auxiliary proteins. Each snRNP contains an snRNA, 7 Sm or LSm proteins and a number of snRNP-specific proteins. The spliceosome assembles de novo on each intron and, through a myriad of RNA-RNA, RNA-protein, and protein-protein interactions, acts to excise each intron and ligate the exons.

Spliceosome assembly requires specific recognition of splice site signals: the 5′ splice site (5′ss) at the 5′ end of the intron, the 3′ splice site (3′ss) at the 3′ end, and branch point site located 18-40 nucleotides upstream of the 3′ss.

As the first step of RNA splicing, U1 snRNP recognizes the 5′ss by base pairing between U1 snRNA and the 5′ss exon-intron junction; in the meanwhile, U2 auxiliary factor 65 (U2AF65) binds to a pyrimidine-rich sequence and interacts with the branch point site binding protein (SF1), assisting its binding to the branch point site, while U2 auxiliary factor 35 (U2AF35) binds to the 3′ss, thus the “Early” or “E” complex is formed. Subsequently, with the assistance of U2AF, U2 snRNP replaces SF1 and binds to the branch site by base pairing to form the A complex, a process that involves a large number of splicing factors. The subsequent binding of the preassembled U4/U6.U5 tri-snRNP allows formation of the fully assembled spliceosomal B complex, which is converted to the catalytically active B* complex through extensive structural and compositional remodeling. During this process, U4 and U6 snRNAs, extensively base-paired in tri-snRNP are unwound, U1 and U4 snRNPs are released, U6 snRNP binds to U2 snRNP by base pairing between U6 and U2 snRNAs, and many new proteins join the spliceosome. This leads to the formation of a highly structured RNA network between U2, U5 and U6 snRNAs and the 5′SS and branch point site sequences in the pre-mRNA. The extensively base-paired U2-U6 snRNAs harbor catalytic magnesium ions and position the branch point site and 5′ss for the first trans-esterification reaction, which produces exon 1 and lariat intron-exon 2 intermediates. Further remodeling to C complex enables U5 snRNA loop 1 to align exons 1 and 2 for nucleophilic attack of exon 1 at the 3′SS, yielding spliced mRNA and lariat intron products. Finally the spliceosome is disassembled before the next round of splicing.

It is estimated that 35-50% of human diseases are caused by gene splicing abnormalities, including some cancers, genetic diseases, etc., and purposeful regulation or modulation of the RNA splicing process provides a new concept for the treatment of these diseases.

The disclosures of all publications, patents, patent applications and published patent applications referred to herein are hereby incorporated herein by reference in their entirety.

### BRIEF SUMMARY

In some embodiments, provided herein is a method of modulating splicing of a target exon in a pre-mRNA in a host cell, the pre-mRNA comprising the target exon flanked by a 5′ intron and a 3′ intron, wherein the method comprises introducing a deaminase-recruiting RNA (dRNA) or a construct comprising a nucleic acid encoding the dRNA into the host cell, wherein: (1) dRNA comprises a targeting RNA sequence that is at least partially complementary to a target sequence in the pre-mRNA comprising a portion of the target exon, a portion of the 5′ intron, and/or a portion of the 3′ intron; and (2) the dRNA recruits an adenosine deaminase acting on RNA (ADAR) to the target exon, the 5′ intron, and/or the 3′ intron; wherein the recruitment of the ADAR modulate the splicing of the target exon in the pre-mRNA.

In some embodiments, the recruitment of the ADAR blocks access of one or more splicing factors to a splicing site for the target exon, thereby modulating the splicing of the target exon in the pre-mRNA. In some embodiments, the recruitment of the ADAR does not deaminate a target adenosine in the 5′ intron or the 3′ intron. In any of the embodiments herein, the recruitment of the ADAR can deaminate a target adenosine in the 5′ intron or the 3′ intron, thereby modulating the splicing of the target exon in the pre-mRNA.

In any of the embodiments herein, the pre-mRNA can be spliced in the host cell to form a mature mRNA not including the target exon. In any of the embodiments herein, the pre-mRNA can be spliced in the host cell to form a mature mRNA including the target exon.

In any of the embodiments herein, the target sequence can comprise a 3′ splice site of the 5′ intron comprising an AG splicing sequence. In some embodiments, the targeting sequence comprises a mismatch nucleotide opposite to the adenosine in the AG splicing sequence, thereby inducing deamination at the adenosine upon recruitment of the ADAR. In some embodiments, the targeting sequence does not comprise a mismatch nucleotide opposite to the adenosine in the AG splicing sequence.

In any of the embodiments herein, the target sequence can comprise a branch point of the 5′ intron. In some embodiments, the targeting RNA sequence comprises a mismatch nucleotide opposite to the adenosine in the branch point, thereby inducing deamination at the adenosine upon recruitment of the ADAR. In some embodiments, the targeting RNA sequence does not comprise a mismatch nucleotide opposite to the adenosine in the branch point.

In any of the embodiments herein, the target sequence can comprise a pyrimidine-rich sequence of the 5′ intron. In any of the embodiments herein, the target sequence can comprise an exonic splicing enhancer sequence of the target exon.

In any of the embodiments herein, the targeting RNA sequence can be more than about 30 nucleotides in length. In some embodiments, the targeting RNA sequence is about 70 to about 150 nucleotides in length.

In any of the embodiments herein, the dRNA can further comprise a 3′ ligation sequence and a 5′ ligation sequence.

In any of the embodiments herein, the dRNA can be a circRNA. In any of the embodiments herein, the dRNA can be a linear RNA. In some embodiments, the dRNA is a linear RNA that forms a circRNA within the host cell.

In any of the embodiments herein, the method can comprise introducing a construct comprising a nucleic acid encoding the dRNA into the host cell. In some embodiments, the construct further comprises a 3′ twister ribozyme sequence linked to the 3′ end of the nucleic acid encoding the dRNA and a 5′ twister ribozyme sequence linked to the 5′ end of the nucleic acid encoding the dRNA.

In any of the embodiments herein, the construct can be a viral vector or a plasmid. In some embodiments, the construct is an AAV vector, lentiviral vector, adenoviral vector, RNA replicon, pox virus vector, enteric virus vector, Venezuelan Equine Encephalitis virus vector, Semliki Forest Virus vector, or Tobacco Mosaic Virus vector.

In any of the embodiments herein, the dRNA further comprises an ADAR recruiting domain. In any of the embodiments herein, the dRNA can but does not need to comprise an ADAR recruiting domain.

In any of the embodiments herein, the method can comprise introduction of at least two dRNAs or constructs comprising a nucleic acid encoding the dRNA into the host cell, each comprising a targeting RNA sequence that is at least partially complementary to a different target sequence in the pre-mRNA.

In any of the embodiments herein, the dRNA can but does not need to be chemically modified. In any of the embodiments herein, the dRNA can be chemically modified.

In any of the embodiments herein, the pre-mRNA can be from a gene selected from the group consisting of dystrophin, RELA, Trp53, PPIB, STAT3, SMN1, CFTR, beta-globin, Factor VIII, Factor IX, TP53, mesenchymal epithelial transition factor receptor (MET), KRAS, RAB7A, ABCA4, SERPINA1, HEXA, LRRK2, SNCA, DMD, APP, MAPT, CFTR, ALAS1, ATP7B, HFE, LIP A, CEP290, USH2A, CD274, HTT, COL7A1, MS4A, SGCG, ATXN3, NOTCH3, COL4A5, NF1, PCSK9 start site, SCNN1A, a collagen VI alpha 1 gene (COL6A1), a myotubular myopathy 1 gene (MTM1), a dysferlin gene (DYSF), a laminin-alpha 2 gene (LAMA2), an emery-dreyfuss muscular dystrophy gene (EMD), a calpain 3 gene (CAPN3), and superoxide dismutase 1 (SOD1). In some embodiments, (1) the pre-mRNA is from a dystrophin gene, and wherein the target exon is selected from the group consisting of exons 3, 4, 5, 8, 10-16, 19-40, 42-44, 46, 47, and 50-53; (2) the pre-mRNA is from an SMN1 gene and wherein the target exon is exon 7; (3) the pre-mRNA is from a CFTR gene, and wherein the target exon is selected from the group consisting of exon 10, 11, 12, and 13; (4) the pre-mRNA is from a beta-globin gene and wherein the target exon is selected from the group consisting of exon 51, 44, 45, and 27; (5) the pre-mRNA is from a Factor VIII gene, and wherein the target exon is selected from the group consisting of exon 5, 6, 7, and 8; (6) the pre-mRNA is from a Factor IX gene, and wherein the target exon is exon 2; (7) the pre-mRNA is from a RELA gene, and wherein the target exon is exon 7; (8) the pre-mRNA is from a Trp53 genes, and wherein the target exon is selected from the group consisting of exons 2 and 5; (9) the pre-mRNA is from a PPIB gene, and wherein the target exon is exon 3.

In any of the embodiments herein, the host cell can be an eukaryotic cell.

In some embodiments, provided herein is a method of treating or preventing a disease of an individual, comprising inducing exon skipping in a pre-mRNA of a host cell in the individual according to the method of any of the embodiments herein.

In some embodiments, the pre-mRNA is from a mutant dystrophin gene, and the disease or condition is Duchenne muscular dystrophy (DMD); the pre-mRNA is from a mutant SMN1 gene, and the disease or condition is Spinal muscular atrophy (SMA); the pre-mRNA is from a mutant CFTR gene, and the disease or condition is Cystic fibrosis (CF); the pre-mRNA is from a mutant beta-globin gene, and the disease or condition is Beta-thalassemia; or the pre-mRNA is from a mutant Factor VIII or Factor IX gene, and the disease or condition is hemophilia.

In certain embodiments, the pre-mRNA is a pre-mRNA in need of modulation of RNA splicing, such as a pre-mRNA of a dystrophin gene or an oncogene, such as a TP53 gene, a MET gene, a KRAS gene, or STAT3, or a gene selected from the group consisting of RAB7A, ABCA4, SERPINA1, HEXA, LRRK2, SNCA, DMD, APP, MAPT, CFTR, ALAS1, ATP7B, HFE, LIP A, SOD1, CEP290, USH2A, CD274, HTT, COL7A1, MS4A, SGCG, DYSF, COL6A1, MTM1, LAMA2, EMD, CAPN3, ATXN3, NOTCH3, COL4A5, NF1, PCSK9 start site, or SCNN1A start site, or a fragment or any combination thereof.

In any of the embodiments herein, the dRNA or construct encoding the dRNA is introduced into the individual intravenously.

In some embodiments, provided herein is a ribonucleic acid (RNA) molecule, comprising a targeting RNA sequence at least partially complementary to a target RNA sequence, wherein the target RNA sequence comprises one or more splice sites, such as at least one of a 5′ splice site (5′ ss), a polypyrimidine tract, a branchpoint and a 3′ splice site (3′ ss) of a pre-mRNA, and the dRNA molecule is capable of recruiting an ADAR to the target RNA sequence. In some embodiments, the RNA molecule is a circular RNA. In any of the embodiments herein, the RNA molecule can further comprise a 3′ ligation sequence and a 5′ ligation sequence. In any of the embodiments herein, the RNA molecule can be a linear RNA capable of forming a circular RNA, preferably, the RNA molecule can be capable of being circularized by RNA ligase RtcB in a host cell.

In any of the embodiments herein, the targeting RNA sequence can have a length of at least 30 nucleotides, such as at least 40, 50, 60, 70, 80, 90, 100, 110, 120, 130, 140, 150, 160, 170, 180, 190, 200, or more nucleotides.

In any of the embodiments herein, the target RNA sequence can comprise at least one, two, or three of the 3′ splice site (3′ ss), the polypyrimidine tract, and the branchpoint. In certain embodiments, the target RNA sequence comprises at least one, two, three, or four of the 5′ splice site (5′ ss), 3′ splice site (3′ ss), the polypyrimidine tract, and the branchpoint.

In certain embodiments,
a. the 3′ ss comprises the nucleotide sequence of YAG,
b. the polypyrimidine tract has a length of 4-25 nucleotides, such as 4, 5, 6, 7, 8, 9, 10, 11, 12, 13, 14, 15, 16, 17, 18, 19, 20, 21, 22, 23, 24 or 25 nucleotides, and/or
c. the branchpoint comprises the nucleotide sequence of YUNAY, such as CURAY, YNCURAY, YNCURAC or YNYURAY; where Y is independently a pyrimidine, N is any nucleotide, and R is a purine.

In certain embodiments, the target RNA sequence comprises, in 5′ to 3′ direction:
a. the branchpoint comprising the nucleotide sequence of YNYURAY and the polypyrimidine tract having a length of 10-20 nucleotides;
b. the polypyrimidine tract having a length of 10-20 nucleotides and the 3′ ss comprising the nucleotide sequence of YAG; or
c. the branchpoint comprising the nucleotide sequence of YNYURAY, the polypyrimidine tract having a length of 10-20 nucleotides; and the 3′ ss comprising the nucleotide sequence of YAG, optionally, the target RNA sequence further comprises a 5′ splice site (5′ ss).

In some embodiments, provided herein is a system for modulating splicing of a target exon in a pre-mRNA in a host cell, the pre-mRNA comprising the target exon flanked by a 5′ intron and a 3′ intron, wherein the system comprises a dRNA or a construct comprising a nucleic acid encoding the dRNA, wherein: (1) dRNA comprises a targeting RNA sequence that is at least partially complementary to a target sequence in the pre-mRNA comprising a portion of the target exon, a portion of the 5′ intron, and/or a portion of the 3′ intron; and (2) the dRNA is capable of recruiting an adenosine deaminase acting on RNA (ADAR) to the target exon, the 5′ intron, and/or the 3′ intron.

In some embodiments, the target sequence comprises a 3′ splice site of the 5′ intron comprising an AG splicing sequence. In some embodiments, the targeting sequence comprises a mismatch nucleotide opposite to the adenosine in the AG splicing sequence. In some embodiments, the targeting sequence does not comprise a mismatch nucleotide opposite to the adenosine in the AG splicing sequence.

In any of the embodiments herein, the target sequence can comprise a branch point of the 5′ intron. In some embodiments, the targeting RNA sequence comprises a mismatch nucleotide opposite to the adenosine in the branch point. In some embodiments, the targeting RNA sequence does not comprise a mismatch nucleotide opposite to the adenosine in the branch point.

In any of the embodiments herein, the target sequence can comprise a pyrimidine-rich sequence of the 5′ intron. In any of the embodiments herein, the target sequence can comprise an exonic splicing enhancer sequence of the target exon.

In any of the embodiments herein, the targeting RNA sequence can be more than about 30 nucleotides in length. In some embodiments, the targeting RNA sequence is about 70 to about 150 nucleotides in length.

In any of the embodiments herein, the dRNA can further comprise a 3′ ligation sequence and a 5′ ligation sequence.

In any of the embodiments herein, the dRNA can be a circRNA. In any of the embodiments herein, the dRNA can be a linear RNA. In some embodiments, the dRNA is a linear RNA that is capable of forming a circRNA within a host cell.

In any of the embodiments herein, the system can comprise a construct comprising a nucleic acid encoding the dRNA. In some embodiments, the construct further comprises a 3′ twister ribozyme sequence linked to the 3′ end of the nucleic acid encoding the dRNA and a 5′ twister ribozyme sequence linked to the 5′ end of the nucleic acid encoding the dRNA.

In any of the embodiments herein, the construct can be a viral vector or a plasmid. In some embodiments, the construct is an AAV vector, lentiviral vector, adenoviral vector, RNA replicon, pox virus vector, enteric virus vector, Venezuelan Equine Encephalitis virus vector, Semliki Forest Virus vector, or Tobacco Mosaic Virus vector.

In any of the embodiments herein, the dRNA can further comprise an ADAR recruiting domain. In any of the embodiments herein, the dRNA can but does not need to comprise an ADAR recruiting domain.

In any of the embodiments herein, the system can comprise at least two dRNAs or constructs comprising a nucleic acid encoding the dRNA, each comprising a targeting RNA sequence that is at least partially complementary to a different target sequence in the pre-mRNA.

In any of the embodiments herein, the dRNA can but does not need to be chemically modified. In any of the embodiments herein, the dRNA can be chemically modified.

In any of the embodiments herein, the pre-mRNA can be from a gene selected from the group consisting of dystrophin, TP53 gene, a MET gene, a KRAS gene, or STAT3, or a gene selected from the group consisting of RAB7A, ABCA4, SERPINA1, HEXA, LRRK2, SNCA, DMD, APP, MAPT, CFTR, ALAS1, ATP7B, HFE, LIP A, SOD1, CEP290, USH2A, CD274, HTT, COL7A1, MS4A, SGCG, DYSF, COL6A1, MTM1, LAMA2, EMD, CAPN3, ATXN3, NOTCH3, COL4A5, NF1, PCSK9 start site, or SCNN1A start site, or a fragment or any combination thereof.

In some embodiments, (1) the pre-mRNA is from a dystrophin gene, and wherein the target exon is selected from the group consisting of exons 3, 4, 5, 8, 10-16, 19-40, 42-44, 46, 47, and 50-53; (2) the pre-mRNA is from an SMN1 gene and wherein the target exon is exon 7; (3) the pre-mRNA is from a CFTR gene, and wherein the target exon is selected from the group consisting of exon 10, 11, 12, and 13; (4) the pre-mRNA is from a beta-globin gene and wherein the target exon is selected from the group consisting of exon 51, 44, 45, and 27; (5) the pre-mRNA is from a Factor VIII gene, and wherein the target exon is selected from the group consisting of exon 5, 6, 7, and 8; or (6) the pre-mRNA is from a Factor IX gene, and wherein the target exon is exon 2; (7) the pre-mRNA is from a RELA gene, and wherein the target exon is exon 7; (8) the pre-mRNA is from a Trp53 genes, and wherein the target exon is selected from the group consisting of exons 2 and 5; (9) the pre-mRNA is from a PPIB gene, and wherein the target exon is exon 3.

In any of the embodiments herein, the host cell can be an eukaryotic cell.

In some embodiments, provided herein is use of the system of any one of the embodiments herein for the manufacture of a medicament for treating or preventing a disease in an individual.

In some embodiments, the pre-mRNA is from a mutant dystrophin gene, and the disease or condition is Duchenne muscular dystrophy (DMD); the pre-mRNA is from a mutant SMN1 gene, and the disease or condition is Spinal muscular atrophy (SMA); the pre-mRNA is from a mutant CFTR gene, and the disease or condition is Cystic fibrosis (CF); the pre-mRNA is from a mutant beta-globin gene, and the disease or condition is Beta-thalassemia; or the pre-mRNA is from a mutant Factor VIII or Factor IX gene, and the disease or condition is hemophilia.

In any of the embodiments herein, the dRNA or construct encoding the dRNA can be suitable for intravenous administration.

It is to be understood that one, some, or all of the properties of the various embodiments described herein may be combined to form other embodiments of the present invention. These and other embodiments of the invention are further described by the detailed description that follows.

### BRIEF DESCRIPTION OF THE DRAWINGS

FIGS. 1A-1D show modulation of RELA and Trp53 pre-mRNA splicing in vitro. FIG. 1A shows the exon skipping efficiency for RELA. FIG. 1B shows that exon 7 was skipped in the mature RELA mRNA using circular arRNA. FIG. 1C shows the exon skipping efficiency for Trp53. FIG. 1D shows exon skipping efficiency in HEK293T cells transfected by different dosage of circ-arRNA. The number of cells transfected was 2×10⁵.

FIGS. 2A-2F show modulation of PPIB and RELA pre-mRNA splicing in vitro in HEK293T cells and HEK293T ADAR1 knockout cells. FIG. 2A shows the exon skipping efficiency for PPIB was lower in ADAR1 knockout cells compared to in HEK293T cells. FIG. 2B shows the exon skipping efficiency for RELA was lower in ADAR1 knockout cells compared to in HEK293T cells. FIG. 2C shows the presence of circ-arRNA in HEK293T cells was confirmed via Urea-PAGE. 150 nt and 300 nt markers are also shown on the figure. FIG. 2D shows exon skipping efficiency at exon 5 in monkey DMD with no ADAR1 or with ADAR1 in its various forms. HAE represents deaminase active form of ADAR1 while QAA and HAG are inactive forms. FIG. 2E shows RNA editing efficiency at exon 5 in monkey DMD with no ADAR1 or with ADAR1 in its various forms. HAE represents deaminase active form of ADAR1 while QAA and HAG are inactive forms. FIG. 2F shows heat map of the rates of editing in dsRNA region covered by circ-arRNA151. SRp55/SC35 and SF2/ASF represent Exon Splicing Enhancer sequences (ESE).

FIG. 3A-3B show the exon skipping efficiencies using different promoters, different arRNA lengths, and/or with or without a Tornado expression system. FIG. 3A shows the exon skipping efficiencies for the DMD gene in vitro using different promoters, different arRNA lengths, and and/or with or without a Tornado expression system. FIG. 3B shows different exon skipping efficiencies for the STAT3 gene in vitro using linear or circular arRNA and/or using different promoters.

FIGS. 4A-4E show modulation of DMD splicing in vivo. FIG. 4A shows the exon skipping efficiencies in the tissues 8 weeks after injection. FIG. 4B shows the relative expression of the circular arRNAs in the tissues normalized to GAPDH 8 weeks after injection. FIG. 4C shows the editing rates in the tissues measured by next-generation sequencing 8 weeks after injection. FIG. 4D shows exon skipping efficiency of DMD in various muscle areas at different time points in monkeys. FIG. 4E shows correlation (Pearson R) among exon skipping efficiency of DMD, circ-arRNA abundance and editing rates of A at 5A site in monkeys.

FIGS. 5A-5D show effects of arRNA length and targeting site on modulation of DMD splicing. FIG. 5A shows RT-PCR results of exon skipping of DMD exon 51 by circ-arRNA151 targeting splicing acceptor (SA) of intron 50 or Exon Splicing Enhancer (ESE) in exon 51. FIG. 5B illustrates ESE motifs prediction in mouse DMD exon 51 (top) and designs of circ-arRNAs in different length targeting ESE motifs (bottom white bars). FIG. 5C shows RT-PCR results of exon skipping ratios in DMD exon 51 by circ-arRNAs targeting exon 51 using arRNAs shown in FIG. 5B. FIG. 5D shows efficiency of modulation of skipping of exon 51 by circ-arRNAs of different lengths (via in vitro transcription) at 12 h and 24 h post transfection.

### DETAILED DESCRIPTION

The present description provides an effective method of modulating splicing of pre-mRNAs using specifically designed RNAs, namely, deaminase-recruiting RNAs (“dRNAs”) which are either circular (circRNA) or linear dRNA, optionally a linear dRNA that can form circRNA within a cell. The dRNAs contain a targeting RNA sequence that is at least partially complementary to a portion of a target exon and/or a portion of its flanking intron and recruits an adenosine deaminase acting on RNA (ADAR) to the target exon and/or intron, thereby modulating the splicing of the pre-mRNA. In some embodiments, the modulation of the pre-mRNA splicing allows the pre-mRNA to be spliced to form a mature mRNA not including the target exon (“exon skipping”). In some embodiments, the recruitment of the ADAR blocks access of one or more splicing factors to a splicing site for the target exon, thereby modulating the splicing of the target exon. This can be accomplished, for example, without editing of the pre-mRNA. In some embodiments, on the other hand, the recruitment of the ADAR results in editing of the pre-mRNA to change sites which are critical for splicing, further reducing splicing efficiency of the target exon. Surprisingly, the splicing modulation is much more effective when the dRNA is circRNA or forms circRNA within a cell. It was also observed that the splicing modulation is much more effective above certain threshold length of the targeting RNA sequence.

The present application thus in some embodiments provides a method of modulating splicing of a target exon in a pre-mRNA in a host cell, the pre-mRNA comprising the target exon flanked by a 5′ intron and a 3′ intron, wherein the method comprises introducing a deaminase-recruiting RNA (dRNA) or a construct comprising a nucleic acid encoding the dRNA into the host cell, wherein: (1) dRNA comprises a targeting RNA sequence that is at least partially complementary to a target sequence in the pre-mRNA comprising a portion of the target exon, a portion of the 5′ intron, and/or a portion of the 3′ intron; and (2) the dRNA recruits an ADAR to the target exon, the 5′ intron, and/or the 3′ intron; wherein the recruitment of the ADAR modulate the splicing of the target exon in the pre-mRNA. In some embodiments, the recruitment of the ADAR blocks access of one or more splicing factors to a splicing site for the target exon, thereby modulating the splicing of the target exon in the pre-mRNA. In some embodiments, the recruitment of the ADAR deaminates a target adenosine in the 5′ intron or the 3′ intron, thereby modulating the splicing of the target exon in the pre-mRNA.

In some embodiments, there is provided a method of inducing exon skipping in a pre-mRNA in a host cell, comprising introducing a dRNA or construct comprising a nucleic acid encoding the dRNA into the host cell, wherein the dRNA is circRNA, a linear dRNA, or a linear dRNA that forms a circRNA within the host cell, and wherein the dRNA comprises a targeting RNA sequence that is at least partially complementary to a portion of the target exon and/or a portion of the 5′ intron (or 3′ intron) and recruits an ADAR to the target exon and/or 5′ intron (or 3′ intron).

Also provided are systems for carrying out the methods described herein. Also provided herein are methods and compositions for treating or preventing a disease or condition in an individual using the exon skipping methods described here.

### I. Definitions

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to particular method steps, reagents, or conditions are specifically embraced by the present disclosure and are disclosed herein just as if each and every combination was individually and explicitly disclosed.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The terms “deaminase-recruiting RNA,” “dRNA,” “ADAR-recruiting RNA” and “arRNA” are used herein interchangeably to refer to an engineered RNA capable of recruiting an ADAR to a target exon and/or a flanking 5′ intron.

The terms “polynucleotide,” “nucleotide sequence” and “nucleic acid” are used interchangeably. They refer to a polymeric form of nucleotides of any length, either deoxyribonucleotides or ribonucleotides, or analogs thereof.

The terms “adenine,” “guanine,” “cytosine,” “thymine,” “uracil” and “hypoxanthine” as used herein refer to the nucleobases as such. The terms “adenosine,” “guanosine,” “cytidine,” “thymidine,” “uridine” and “inosine,” refer to the nucleobases linked to the ribose or deoxyribose sugar moiety. The term “nucleoside” refers to the nucleobase linked to the ribose or deoxyribose. The term “nucleotide” refers to the respective nucleobase-ribosyl-phosphate or nucleobase-deoxyribosyl-phosphate. Sometimes the terms adenosine and adenine (with the abbreviation, “A”), guanosine and guanine (with the abbreviation, “G”), cytosine and cytidine (with the abbreviation, “C”), uracil and uridine (with the abbreviation, “U”), thymine and thymidine (with the abbreviation, “T”), inosine and hypo-xanthine (with the abbreviation, “I”), are used interchangeably to refer to the corresponding nucleobase, nucleoside or nucleotide. Sometimes the terms nucleobase, nucleoside and nucleotide are used interchangeably, unless the context clearly requires differently.

The term “introducing” or “introduction” used herein means delivering one or more polynucleotides, such as dRNAs or one or more constructs including vectors as described herein, one or more transcripts thereof, to a host cell. The methods of the present application can employ many delivery systems, including but not limited to, viral, liposome, electroporation, microinjection and conjugation, to achieve the introduction of the dRNA or construct as described herein into a host cell. Conventional viral and non-viral based gene transfer methods can be used to introduce nucleic acids into mammalian cells or target tissues. Such methods can be used to administer nucleic acids encoding dRNA of the present application to cells in culture, or in a host organism. Non-viral vector delivery systems include DNA plasmids, RNA (e.g. a transcript of a construct described herein), naked nucleic acid, and nucleic acid complexed with a delivery vehicle, such as a liposome. Viral vector delivery systems include DNA and RNA viruses, which have either epitomal or integrated genomes for delivery to the host cell.

“pre-mRNA” used in the present application refers to the primary transcript of a gene that can contain intron and exons and requires further splicing to produce mature mRNA molecules containing only exons.

The terms “Group I intron” and “Group I catalytic intron” are used interchangeably to refer to a self-splicing ribozyme that can catalyze its own excision from an RNA precursor. Group I introns comprise two fragments, the 5′ catalytic Group I intron fragment and the 3′ catalytic Group I intron fragment, which retain their folding and catalytic function (i.e., self-splicing activity). In its native environment, the 5′ catalytic Group I intron fragment is flanked at its 5′ end by a 5′ exon, which comprises a 5′ exon sequence that is recognized by the 5′ catalytic Group I intron fragment; and the 3′ catalytic Group I intron fragment is flanked at its 3′ end by a 3′ exon, which comprises a 3′ exon sequence that is recognized by the 3′ catalytic Group I intron fragment.

In the context of the present application, “target sequence” refers to a sequence in a pre-mRNA to which a dRNA sequence is designed to have perfect complementarity or substantial complementarity, and hybridization between the target sequence and the dRNA forms a double stranded RNA (dsRNA) region containing a target adenosine, which recruits an ADAR which optionally deaminates the target adenosine. In some embodiments, the ADAR is naturally present in a host cell, such as a eukaryotic cell (preferably, a mammalian cell, more preferably, a human cell). In some embodiments, the ADAR is introduced into the host cell.

As used herein, “complementarity” refers to the ability of a nucleic acid to form hydrogen bond(s) with another nucleic acid by traditional Watson-Crick base-pairing. A percent complementarity indicates the percentage of residues in a nucleic acid molecule which can form hydrogen bonds (i.e., Watson-Crick base pairing) with a second nucleic acid (e.g., about 5, 6, 7, 8, 9, 10 out of 10, being about 50%, 60%, 70%, 80%, 90%, and 100% complementary respectively). “Perfectly complementary” means that all the contiguous residues of a nucleic acid sequence form hydrogen bonds with the same number of contiguous residues in a second nucleic acid sequence. “Substantially complementary” as used herein refers to a degree of complementarity that is at least about any one of 70%, 75%, 80%, 85%, 90%, 95%, 97%, 98%, 99%, or 100% over a region of about 40, 50, 60, 70, 80, 100, 150, 200, 250 or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay,” Elsevier, N,Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. A sequence capable of hybridizing with a given sequence is referred to as the “complement” of the given sequence.

As used herein, the terms “including,” “containing,” and “comprising” are used in their open, non-limiting sense. It is also understood that aspects and embodiments of the invention described herein may include “consisting” and/or “consisting essentially of” aspects and embodiments.

It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

As used herein, a “carrier” includes pharmaceutically acceptable carriers, excipients, or stabilizers that are nontoxic to the cell or mammal being exposed thereto at the dosages and concentrations employed. Often the physiologically acceptable carrier is an aqueous pH buffered solution. Non-limiting examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

As used herein, the term “effective amount” or “therapeutically effective amount” of a substance is at least the minimum concentration required to affect a measurable improvement or prevention of a particular disorder. An effective amount herein may vary according to factors such as the disease state, age, sex, and weight of the patient, and the ability of the substance to elicit a desired response in the individual. An effective amount is also one in which any toxic or detrimental effects of the treatment are outweighed by the therapeutically beneficial effects. An effective amount can be administered in one or more administrations. For purposes of this disclosure, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

A “host cell” as described herein refers to any cell type that can be used as a host cell provided it can be modified as described herein. For example, the host cell may be a host cell with endogenously expressed ADAR, or may be a host cell into which an ADAR is introduced by a known method in the art. For example, the host cell may be a prokaryotic cell, a eukaryotic cell or a plant cell. In some embodiments, the host cell is derived from a pre-established cell line, such as mammalian cell lines including human cell lines or non-human cell lines. In some embodiments, the host cell is derived from an individual, such as a human individual.

A “recombinant AAV vector (rAAV vector)” refers to a polynucleotide vector comprising one or more heterologous sequences (i.e., nucleic acid sequence not of AAV origin) that are flanked by at least one, and in embodiments two, AAV inverted terminal repeat sequences (ITRs). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. An rAAV vector can be in any of a number of forms, including, but not limited to, plasmids, linear artificial chromosomes, complexed with lipids, encapsulated within liposomes, and encapsidated in a viral particle, particularly an AAV particle. A rAAV vector can be packaged into an AAV virus capsid to generate a “recombinant adeno-associated viral particle (rAAV particle)”.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments, etc.

A “subject,” “patient” or “individual” includes a mammal, such as a human or other animal, and typically is human. In some embodiments, the subject, e.g., patient, to whom the therapeutic agents and compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent, a dog, a cat, a farm animal, such as a cow or a horse, etc.

As used herein, the term “treatment” refers to clinical intervention designed to have beneficial and desired effects to the natural course of the individual or cell being treated during the course of clinical pathology. For the purpose of this disclosure, desirable effects of treatment include, without limitation, decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, increasing cancer cell-killing, decreasing symptoms resulting from the disease, preventing spread of diseases, preventing recurrence of disease, increasing the quality of life of those suffering from the disease, decreasing the dose of other medications required to treat the disease, delaying the progression of the disease, and/or prolonging survival of individuals.

### II. Methods of Modulating RNA Splicing

Provided herein are method of modulating splicing of a target exon in a target RNA (such as a pre-mRNA) in a host cell, the target RNA comprising the target exon flanked by a 5′ intron and a 3′ intron, wherein the method comprises introducing a deaminase-recruiting RNA (dRNA) or a construct comprising a nucleic acid encoding the dRNA into the host cell, wherein: (1) dRNA comprises a targeting RNA sequence that is at least partially complementary to a target sequence in the target RNA (such as pre-mRNA) comprising a portion of the target exon, a portion of the 5′ intron, and/or a portion of the 3′ intron; and (2) the dRNA recruits an ADAR to the target exon, the 5′ intron, and/or the 3′ intron; wherein the recruitment of the ADAR modulate the splicing of the target exon in the target RNA (such as pre-mRNA). In some embodiments, the dRNA is a circular RNA (circRNA). In some embodiments, the dRNA is a linear dRNA. In some embodiments the dRNA is a linear dRNA that forms a circRNA within the host cell, and the circRNA recruits the ADAR. In some embodiments, the method comprises introducing the dRNA. In some embodiments, the method comprises introducing the construct comprising a nucleic acid encoding the dRNA. In some embodiments, the targeting RNA sequence is more than about 30 (such as more than about any of 40, 50, 60, 70, 75, 80, 85, 90, 95, 100, 110, 120, 130, 140, 150, 160, 170, 180, 190, or 200) nucleotides in length.

In some embodiments, there is provided a method of modulating splicing of a target exon in a target RNA (such as a pre-mRNA) in a host cell, the target RNA comprising the target exon flanked by a 5′ intron and a 3′ intron, wherein the method comprises introducing a deaminase-recruiting RNA (dRNA) or a construct comprising a nucleic acid encoding the dRNA into the host cell, wherein: (1) dRNA comprises a targeting RNA sequence that is at least partially complementary to a target sequence in the target RNA (such as pre-mRNA) comprising a portion of the target exon, a portion of the 5′ intron, and/or a portion of the 3′ intron; and (2) the dRNA recruits an ADAR to the target exon, the 5′ intron, and/or the 3′ intron; wherein the recruitment of the ADAR blocks access of one or more splicing factors to a splicing site for the target exon, thereby modulating the splicing of the target exon in the target RNA (such as pre-mRNA). In some embodiments, the recruitment of the ADAR does not deaminate a target adenosine in the 5′ intron or the 3′ intron. In some embodiments, the dRNA is a circRNA. In some embodiments, the dRNA is a linear dRNA. In some embodiments the dRNA is a linear dRNA that forms a circRNA within the host cell, and the circRNA recruits the ADAR. In some embodiments, the method comprises introducing the dRNA. In some embodiments, the method comprises introducing the construct comprising a nucleic acid encoding the dRNA. In some embodiments, the targeting RNA sequence is more than about 30 (such as more than about any of 40, 50, 60, 70, 75, 80, 85, 90, 95, 100, 110, 120, 130, 140, 150, 160, 170, 180, 190, or 200) nucleotides in length.

In some embodiments, there is provided a method of modulating splicing of a target exon in a target RNA (such as a pre-mRNA) in a host cell, the target RNA comprising the target exon flanked by a 5′ intron and a 3′ intron, wherein the method comprises introducing a deaminase-recruiting RNA (dRNA) or a construct comprising a nucleic acid encoding the dRNA into the host cell, wherein: (1) dRNA comprises a targeting RNA sequence that is at least partially complementary to a target sequence in the target RNA (such as pre-mRNA) comprising a portion of the target exon, a portion of the 5′ intron, and/or a portion of the 3′ intron; and (2) the dRNA recruits an ADAR to the target exon, the 5′ intron, and/or the 3′ intron; wherein the recruitment of the ADAR deaminates a target adenosine in the 5′ intron or the 3′ intron, thereby modulating the splicing of the target exon in the target RNA (such as pre-mRNA). In some embodiments, the dRNA is a circRNA. In some embodiments, the dRNA is a linear dRNA. In some embodiments the dRNA is a linear dRNA that forms a circRNA within the host cell, and the circRNA recruits the ADAR. In some embodiments, the method comprises introducing the dRNA. In some embodiments, the method comprises introducing the construct comprising a nucleic acid encoding the dRNA. In some embodiments, the targeting RNA sequence is more than about 30 (such as more than about any of 40, 50, 60, 70, 75, 80, 85, 90, 95, 100, 110, 120, 130, 140, 150, 160, 170, 180, 190, or 200) nucleotides in length.

In some embodiments, there is provided a method of modulating splicing of a target exon in a target RNA (such as a pre-mRNA) in a host cell, the target RNA comprising the target exon flanked by a 5′ intron and a 3′ intron, wherein the method comprises introducing a deaminase-recruiting RNA (dRNA) or a construct comprising a nucleic acid encoding the dRNA into the host cell, wherein: (1) dRNA comprises a targeting RNA sequence that is at least partially complementary to a target sequence in the target RNA (such as pre-mRNA) comprising a portion of the target exon, a portion of the 5′ intron, and/or a portion of the 3′ intron; and (2) the dRNA recruits an ADAR to the target exon, the 5′ intron, and/or the 3′ intron; wherein the recruitment of the ADAR blocks access of one or more splicing factors to a splicing site for the target exon and deaminates a target adenosine in the 5′ intron or the 3′ intron, thereby modulating the splicing of the target exon in the target RNA (such as pre-mRNA). In some embodiments, the dRNA is a circRNA. In some embodiments, the dRNA is a linear dRNA. In some embodiments the dRNA is a linear dRNA that forms a circRNA within the host cell, and the circRNA recruits the ADAR. In some embodiments, the method comprises introducing the dRNA. In some embodiments, the method comprises introducing the construct comprising a nucleic acid encoding the dRNA. In some embodiments, the targeting RNA sequence is more than about 30 (such as more than about any of 40, 50, 60, 70, 75, 80, 85, 90, 95, 100, 110, 120, 130, 140, 150, 160, 170, 180, 190, or 200) nucleotides in length.

In some embodiments, there is provided a method of inducing exon skipping in a pre-mRNA in a host cell, the pre-mRNA comprising a target exon flanked by a 5′ intron and a 3′ intron, wherein the method comprises introducing a deaminase-recruiting RNA (dRNA) or a construct comprising a nucleic acid encoding the dRNA into the host cell, wherein: (1) the dRNA comprises a targeting RNA sequence that is at least partially complementary to a target sequence in the pre-mRNA comprising a portion of the target exon and/or a portion of the 5′ intron (or 3′ intron); and (2) the dRNA recruits an ADAR to the target exon and/or 5′ intron (or 3′ intron); and wherein the pre-mRNA is spliced in the host cell to form a mature mRNA not including the target exon. In some embodiments, the dRNA is a circRNA. In some embodiments, the dRNA is a linear dRNA. In some embodiments the dRNA is a linear dRNA that forms a circRNA within the host cell, and the circRNA recruits the ADAR. In some embodiments, the method comprises introducing the dRNA. In some embodiments, the method comprises introducing the construct comprising a nucleic acid encoding the dRNA. In some embodiments, the targeting RNA sequence is more than about 30 (such as more than about any of 40, 50, 60, 70, 75, 80, 85, 90, 95, 100, 110, 120, 130, 140, 150, 160, 170, 180, 190, or 200) nucleotides in length.

In some embodiments, there is provided a method of modulating splicing in a pre-mRNA in a host cell, the pre-mRNA comprising a target exon flanked by a 5′ intron and a 3′ intron, wherein the method comprises introducing a deaminase-recruiting RNA (dRNA) or a construct comprising a nucleic acid encoding the dRNA into the host cell, wherein: (1) the dRNA is a circRNA, a linear dRNA, or a linear dRNA that forms a circRNA within the host cell; (2) the dRNA comprises a targeting RNA sequence that is at least partially complementary to a target sequence in the pre-mRNA comprising a 3′ splice site of the 5′ intron comprising an AG splicing sequence; and (3) the dRNA (directly or via circRNA formed from the dRNA) recruits an ADAR to the target exon and/or 5′ intron; thereby modulating splicing of the pre-mRNA. In some embodiments, there is provided a method of inducing exon skipping in a pre-mRNA in a host cell, the pre-mRNA comprising a target exon flanked by a 5′ intron and a 3′ intron, wherein the method comprises introducing a deaminase-recruiting RNA (dRNA) or a construct comprising a nucleic acid encoding the dRNA into the host cell, wherein: (1) the dRNA is a circRNA, a linear dRNA, or a linear dRNA that forms a circRNA within the host cell; (2) the dRNA comprises a targeting RNA sequence that is at least partially complementary to a target sequence in the pre-mRNA comprising a 3′ splice site of the 5′ intron comprising an AG splicing sequence; and (3) the dRNA (directly or via circRNA formed from the dRNA) recruits an ADAR to the target exon and/or 5′ intron; and wherein the pre-mRNA is spliced in the host cell to form a mature mRNA not including the target exon. In some embodiments, the targeting sequence comprises a mismatch nucleotide opposite to the adenosine in the AG splicing sequence, thereby inducing deamination at the adenosine upon recruitment of the ADAR. In some embodiments, the target sequence comprises a pyrimidine-rich sequence of the 5′ intron. In some embodiments, the target sequence comprises an exonic splicing enhancer sequence of the target exon. In some embodiments, the targeting RNA sequence is more than about 30 (such as more than about any of 40, 50, 60, 70, 75, 80, 85, 90, 95, 100, 110, 120, 130, 140, 150, 160, 170, 180, 190, or 200) nucleotides in length.

In some embodiments, there is provided a method of modulating splicing in a pre-mRNA in a host cell, the pre-mRNA comprising a target exon flanked by a 5′ intron and a 3′ intron, wherein the method comprises introducing a construct comprising a nucleic acid encoding a dRNA into the host cell, wherein: (1) the dRNA forms a circRNA within the host cell; (2) the dRNA comprises a targeting RNA sequence that is at least partially complementary to a target sequence in the pre-mRNA comprising a 3′ splice site of the 5′ intron comprising an AG splicing sequence; and (3) the circRNA recruits an ADAR to the target exon and/or 5′ intron; thereby modulating the splicing of the pre-mRNA. In some embodiments, there is provided a method of inducing exon skipping in a pre-mRNA in a host cell, the pre-mRNA comprising a target exon flanked by a 5′ intron and a 3′ intron, wherein the method comprises introducing a construct comprising a nucleic acid encoding a dRNA into the host cell, wherein: (1) the dRNA forms a circRNA within the host cell; (2) the dRNA comprises a targeting RNA sequence that is at least partially complementary to a target sequence in the pre-mRNA comprising a 3′ splice site of the 5′ intron comprising an AG splicing sequence; and (3) the circRNA recruits an ADAR to the target exon and/or 5′ intron; and wherein the pre-mRNA is spliced in the host cell to form a mature mRNA not including the target exon. In some embodiments, the targeting sequence comprises a mismatch nucleotide opposite to the adenosine in the AG splicing sequence, thereby inducing deamination at the adenosine upon recruitment of the ADAR. In some embodiments, the target sequence comprises a pyrimidine-rich sequence of the 5′ intron. In some embodiments, the target sequence is comprises an exonic splicing enhancer sequence of the target exon. In some embodiments, the targeting RNA sequence is more than about 30 (such as more than about any of 40, 50, 60, 70, 75, 80, 85, 90, 95, 100, 110, 120, 130, 140, 150, 160, 170, 180, 190, or 200) nucleotides in length. In some embodiments, the construct is a viral vector (such as an AAV vectors).

In some embodiments, there is provided a method of modulating splicing in a pre-mRNA in a host cell, the pre-mRNA comprising a target exon flanked by a 5′ intron and a 3′ intron, wherein the method comprises introducing a deaminase-recruiting RNA (dRNA) or a construct comprising a nucleic acid encoding the dRNA into the host cell, wherein: (1) the dRNA is a circRNA, a linear dRNA, or a linear dRNA that forms a circRNA within the host cell; (2) the dRNA comprises a targeting RNA sequence that is at least partially complementary to a target sequence in the pre-mRNA comprising a branch point of the 5′ intron; and (3) the dRNA (directly or via circRNA formed therefrom) recruits an ADAR to the target exon and/or 5′ intron, thereby modulating the splicing of the pre-mRNA. In some embodiments, there is provided a method of inducing exon skipping in a pre-mRNA in a host cell, the pre-mRNA comprising a target exon flanked by a 5′ intron and a 3′ intron, wherein the method comprises introducing a deaminase-recruiting RNA (dRNA) or a construct comprising a nucleic acid encoding the dRNA into the host cell, wherein: (1) the dRNA is a circRNA, a linear dRNA, or a linear dRNA that forms a circRNA within the host cell; (2) the dRNA comprises a targeting RNA sequence that is at least partially complementary to a target sequence in the pre-mRNA comprising a branch point of the 5′ intron; and (3) the dRNA (directly or via circRNA formed therefrom) recruits an ADAR to the target exon and/or 5′ intron; and wherein the pre-mRNA is spliced in the host cell to form a mature mRNA not including the target exon. In some embodiments, the target sequence comprises a branch point sequence comprising YNYURAY, wherein U is C or U, R is A or G, and Y is C or T. In some embodiments, the targeting RNA sequence comprises a mismatch nucleotide opposite to the adenosine in the branch point, thereby inducing deamination at the adenosine upon recruitment of the ADAR. In some embodiments, the target sequence comprises a pyrimidine-rich sequence of the 5′ intron. In some embodiments, the targeting RNA sequence is more than about 30 (such as more than about any of 40, 50, 60, 70, 75, 80, 85, 90, 95, 100, 110, 120, 130, 140, 150, 160, 170, 180, 190, or 200) nucleotides in length.

In some embodiments, there is provided a method of modulating splicing in a pre-mRNA in a host cell, the pre-mRNA comprising a target exon flanked by a 5′ intron and a 3′ intron, wherein the method comprises introducing a construct comprising a nucleic acid encoding a dRNA into the host cell, wherein: (1) the dRNA forms a circRNA within the host cell; (2) the dRNA comprises a targeting RNA sequence that is at least partially complementary to a target sequence in the pre-mRNA comprising a branch point of the 5′ intron; and (3) the circRNA recruits an ADAR to the target exon and/or 5′ intron, thereby modulating the splicing of the pre-mRNA. In some embodiments, there is provided a method of inducing exon skipping in a pre-mRNA in a host cell, the pre-mRNA comprising a target exon flanked by a 5′ intron and a 3′ intron, wherein the method comprises introducing a construct comprising a nucleic acid encoding a dRNA into the host cell, wherein: (1) the dRNA forms a circRNA within the host cell; (2) the dRNA comprises a targeting RNA sequence that is at least partially complementary to a target sequence in the pre-mRNA comprising a branch point of the 5′ intron; and (3) the circRNA recruits an ADAR to the target exon and/or 5′ intron; and wherein the pre-mRNA is spliced in the host cell to form a mature mRNA not including the target exon. In some embodiments, the target sequence comprises a branch point sequence comprising YNYURAY, wherein U is C or U, R is A or G, and Y is C or T. In some embodiments, the targeting RNA sequence comprises a mismatch nucleotide opposite to the adenosine in the branch point, thereby inducing deamination at the adenosine upon recruitment of the ADAR. In some embodiments, the target sequence comprises a pyrimidine-rich sequence of the 5′ intron. In some embodiments, the target sequence is comprises an exonic splicing enhancer sequence of the target exon. In some embodiments, the targeting RNA sequence is more than about 30 (such as more than about any of 40, 50, 60, 70, 75, 80, 85, 90, 95, 100, 110, 120, 130, 140, 150, 160, 170, 180, 190, or 200) nucleotides in length. In some embodiments, the construct is a viral vector (such as an AAV vectors).

In some embodiments, there is provided a method of modulating splicing in a pre-mRNA in a host cell, the pre-mRNA comprising a target exon flanked by a 5′ intron and a 3′ intron, wherein the method comprises introducing a deaminase-recruiting RNA (dRNA) or a construct comprising a nucleic acid encoding the dRNA into the host cell, wherein: (1) the dRNA is a circRNA or forms a circRNA within the host cell; (2) the dRNA comprises a targeting RNA sequence that is at least partially complementary to a target sequence in the pre-mRNA comprising a 3′ splice site of the 5′ intron comprising an AG splicing sequence and a branch point of the 5′ intron; and (3) the circRNA recruits an ADAR to the target exon and/or 5′ intron; thereby modulating splicing of the pre-mRNA. In some embodiments, there is provided a method of inducing exon skipping in a pre-mRNA in a host cell, the pre-mRNA comprising a target exon flanked by a 5′ intron and a 3′ intron, wherein the method comprises introducing a deaminase-recruiting RNA (dRNA) or a construct comprising a nucleic acid encoding the dRNA into the host cell, wherein: (1) the dRNA is a circRNA or forms a circRNA within the host cell; (2) the dRNA comprises a targeting RNA sequence that is at least partially complementary to a target sequence in the pre-mRNA comprising a 3′ splice site of the 5′ intron comprising an AG splicing sequence and a branch point of the 5′ intron; and (3) the circRNA recruits an ADAR to the target exon and/or 5′ intron; and wherein the pre-mRNA is spliced in the host cell to form a mature mRNA not including the target exon. In some embodiments, the target sequence comprises a branch point sequence comprising YNYURAY, wherein U is C or U, R is A or G, and Y is C or T. In some embodiments, the targeting sequence comprises a mismatch nucleotide opposite to the adenosine in the AG splicing sequence, thereby inducing deamination at the adenosine upon recruitment of the ADAR. In some embodiments, the targeting RNA sequence comprises a mismatch nucleotide opposite to the adenosine in the branch point, thereby inducing deamination at the adenosine upon recruitment of the ADAR. In some embodiments, the target sequence comprises a pyrimidine-rich sequence of the 5′ intron. In some embodiments, the target sequence is comprises an exonic splicing enhancer sequence of the target exon. In some embodiments, the targeting RNA sequence is more than about 30 (such as more than about any of 40, 50, 60, 70, 75, 80, 85, 90, 95, 100, 110, 120, 130, 140, 150, 160, 170, 180, 190, or 200) nucleotides in length.

In some embodiments, there is provided a method of modulating splicing in a pre-mRNA in a host cell, the pre-mRNA comprising a target exon flanked by a 5′ intron and a 3′ intron, wherein the method comprises introducing a nucleic acid encoding a dRNA into the host cell, wherein: (1) the dRNA forms a circRNA within the host cell; (2) the dRNA comprises a targeting RNA sequence that is at least partially complementary to a target sequence in the pre-mRNA comprising a 3′ splice site of the 5′ intron comprising an AG splicing sequence and a branch point of the 5′ intron; and (3) the circRNA recruits an ADAR to the target exon and/or 5′ intron; thereby modulating splicing of the pre-mRNA. In some embodiments, there is provided a method of inducing exon skipping in a pre-mRNA in a host cell, the pre-mRNA comprising a target exon flanked by a 5′ intron and a 3′ intron, wherein the method comprises introducing a nucleic acid encoding a dRNA into the host cell, wherein: (1) the dRNA forms a circRNA within the host cell; (2) the dRNA comprises a targeting RNA sequence that is at least partially complementary to a target sequence in the pre-mRNA comprising a 3′ splice site of the 5′ intron comprising an AG splicing sequence and a branch point of the 5′ intron; and (3) the circRNA recruits an ADAR to the target exon and/or 5′ intron; and wherein the pre-mRNA is spliced in the host cell to form a mature mRNA not including the target exon. In some embodiments, the target sequence comprises a branch point sequence comprising YNYURAY, wherein U is C or U, R is A or G, and Y is C or T. In some embodiments, the targeting sequence comprises a mismatch nucleotide opposite to the adenosine in the AG splicing sequence, thereby inducing deamination at the adenosine upon recruitment of the ADAR. In some embodiments, the targeting RNA sequence comprises a mismatch nucleotide opposite to the adenosine in the branch point, thereby inducing deamination at the adenosine upon recruitment of the ADAR. In some embodiments, the target sequence comprises a pyrimidine-rich sequence of the 5′ intron. In some embodiments, the target sequence is comprises an exonic splicing enhancer sequence of the target exon. In some embodiments, the targeting RNA sequence is more than about 30 (such as more than about any of 40, 50, 60, 70, 75, 80, 85, 90, 95, 100, 110, 120, 130, 140, 150, 160, 170, 180, 190, or 200) nucleotides in length. In some embodiments, the construct is a viral vector (such as an AAV vectors).

In some embodiments, the present application provides a method of modulating splicing of a plurality of target exons (e.g., at least about 2, 3, 4, 5, 10, 20, 50, 100, 1000 or more) in host cells by introducing a plurality of the dRNAs, or one or more constructs encoding the dRNAs, into the host cells. In one aspect, the present application provides a method of inducing skipping of a plurality of target exons (e.g., at least about 2, 3, 4, 5, 10, 20, 50, 100, 1000 or more) in host cells by introducing a plurality of the dRNAs, or one or more constructs encoding the dRNAs, into the host cells.

In some embodiments, the dRNA is a circRNA. The circRNA can be formed using a linear RNA in vitro by a ligase. In some embodiments, the ligase is selected from the group consisting of a T4 DNA ligase (T4 Dnl), a T4 RNA ligase 1 (T4 Rnl1) and a T4 RNA ligase 2 (T4 Rnl2). In some embodiments, the linear RNA comprises a 5′ ligation sequence at the 5′ end of a nucleic acid sequence encoding the circRNA, and a 3′ ligation sequence at the 3′ end of the nucleic acid sequence encoding the circRNA, wherein the 5′ ligation sequence and the 3′ ligation sequence can be ligated to each other via the ligase. In some embodiments, said forming a circRNA comprises: (a) contacting the linear RNA with a single-stranded adaptor nucleic acid comprising from the 5′ end to the 3′ end: a first sequence complementary to the 3′ ligation sequence and a second sequence complementary to the 5′ ligation sequence, and wherein the 5′ ligation sequence and the 3′ ligation sequence hybridize to the single-stranded adaptor nucleic acid to provide a duplex nucleic acid intermediate comprising a single strand break between the 3′ end of the 5′ ligation sequence and the 5′ end of the 3′ ligation sequence; (b) contacting the intermediate with an RNA ligase under a condition that allows ligation of the 5′ ligation sequence to the 3′ ligation sequence to provide a circularized RNA product; and (c) isolating the circularized RNA product, thereby providing the circRNA. In some embodiments, said forming a circRNA comprises: (a) contacting the linear RNA with an RNA ligase under a condition that allows ligation of the 5′ ligation sequence to the 3′ ligation sequence to provide a circularized RNA product; and (b) isolating the circularized RNA product, thereby providing the circRNA. In some embodiments, the method further comprises obtaining the linear RNA by in vitro transcription of a nucleic acid construct comprising a nucleic acid sequence encoding the linear RNA. In some embodiments, the method further comprises purifying the circRNA. Methods for forming circRNAs in vitro are described in WO2021008447A1 and WO2022037692A1, each of which is incorporated herein in its entirety by reference.

In one aspect, the dRNA is a linear RNA that is capable of forming a circRNA. Linear RNA capable of forming circRNA are described in WO2021008447, which is specifically incorporated herein by reference. In some embodiments, the circulation is performed using the Tornado expression system (“Twister-optimized RNA for durable overexpression”) as described in Litke, J. L. & Jaffrey, S. R. Highly efficient expression of circRNA aptamers in cells using autocatalytic transcripts. Nat Biotechnol 37, 667-675 (2019), which is hereby incorporated herein by reference in its entirety. Briefly, Tornado-expressed transcripts contain an RNA of interest flanked by Twister ribozymes. A twister ribozyme is any catalytic RNA sequences that are capable of self-cleavage. The ribozymes rapidly undergo autocatalytic cleavage, leaving termini that are ligated by an RNA ligase.

In some embodiments, the dRNA is introduced by a construct comprising a nucleic acid encoding the dRNA. In some embodiments, the construct further comprises a 3′ twister ribozyme sequence linked to the 3′ end of the nucleic acid encoding the dRNA and a 5′ twister ribozyme sequence linked to the 5′ end of the nucleic acid encoding the dRNA. In some embodiments, the 3′ twister sequence is twister P3 U2A and the 5′ twister sequence is twister P1. In some embodiments, wherein the 5′ twister sequence is twister P3 U2A and the 3′ twister sequence is twister P1. In some embodiments, the dRNA undergoes autocatalytic cleavage. In some embodiments, the catalyzed dRNA product comprises a 5′-hydroxyl group and a 2′,3′-cyclic phosphate at the 3′ terminus. In some embodiments, the catalyzed dRNA product is ligated by ubiquitous endogenous RNA ligase (e.g., RNA ligase RtcB). In some embodiments, the construct is a plasmid or a viral vector.

In some embodiments, the dRNA transcript is also flanked a 5′ and/or 3′ litigation sequences which are then flanked by the 5′-Twister ribozyme and/or 3′-Twister ribozymes, respectively. In some embodiments, the dRNA further comprises a 3′ ligation sequence and a 5′ ligation sequence. In some embodiments, the 3′ ligation sequence and the 5′ ligation sequence are at least partially complementary to each other. In some embodiments, the 3′ ligation sequence and the 5′ ligation sequence are at least about 50%, at least about 55%, at least about 60%, at least about 65%, at least about 70%, at least about 75%, at least about 80%, at least about 85%, at least about 90%, at least about 95%, at least about 96%, at least about 97%, at least about 98% or at least about 99% complementary to each other. In some embodiments, the 3′ ligation sequence and the 5′ ligation sequence are fully complementary to each other.

In some embodiments, the 3′ ligation sequence and the 5′ ligation sequence are independently at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides or at least about 100 nucleotides in length. In some embodiments, the 3′ ligation sequence and the 5′ ligation sequence are independently about 20-30 nucleotides, about 30-40 nucleotides, about 40-50 nucleotides, about 50-60 nucleotides, about 60-70 nucleotides, about 70-80 nucleotides, about 80-90 nucleotides, about 90-100 nucleotides, about 100-125 nucleotides, about 125-150 nucleotides, about 20-50 nucleotides, about 50-100 nucleotides or about 100-150 nucleotides in length.

In some embodiments, the dRNA is circularized by an RNA ligase. Non-limiting examples of RNA ligase include: RtcB, T4 RNA Ligase 1, T4 RNA Ligase 2, Rnl3 and Trl1. In some embodiments, the RNA ligase is expressly endogenously in the host cell. In some embodiments, the RNA ligase is RNA ligase RtcB. In some embodiments, the method further comprises introducing an RNA ligase (e.g., RtcB) into the host cell. In some embodiments, the dRNA is circularized through in vitro enzymatic ligation (e.g., using RNA or DNA ligase) or chemical ligation (e.g., using cyanogen bromide or a similar condensing agent).

In some embodiments, the dRNA is chemically synthesized. In some embodiments, the dRNA comprises one or more modifications, such as 2′-O-methylation and/or phosphorothioation. In some embodiments, the dRNA is a linear dRNA of about 60-200 nucleotides long and comprises one or more modifications (such as 2′-O-methylation and/or phosphorothioation). In some embodiments, the dRNA comprises 2′-O-methylations in the first and last 3 nucleotides and/or phosphorothiations in the first and last 3 internucleotide linkages. In some embodiments, the dRNA comprises 2′-O-methylations in the first and last 3 nucleotides, phosphorothiations in the first and last 3 internucleotide linkages, and 2′-O-methylations in one or more uridines, for example on all uridines. Chemically modified dRNAs are provided in WO2022/150974, which is specifically incorporated herein by reference.

In some embodiments when deamination at a target adenosine is desired, the dRNA comprises 2′-O-methylations in the first and last 3 nucleotides, phosphorothiations in the first and last 3 internucleotide linkages, 2′-O-methylations in a single or multiple or all uridines, and a modification in the nucleotide opposite to the target adenosine, and/or one or two nucleotides most adjacent to the nucleotide opposite to the target adenosine. In certain embodiments, the modification in the nucleotide opposite to the target adenosine, and/or one or two nucleotides most adjacent to the nucleotide opposite to the target adenosine is a 2′-O-methylation. In certain embodiments, the modification in the nucleotide opposite to the target adenosine, and/or one or two nucleotides most adjacent to the nucleotide opposite to the target adenosine is a phosphorothiation linkage, such as a 3′-phosphorothiation linkage. In certain embodiments, the dRNA comprises 2′-O-methylations in the first and last 3 nucleotides, phosphorothiations in the first and last 3 internucleotide linkages, 2′-O-methylations in all uridines, and a 2′-O-methylation in the nucleotide adjacent to the 3′ terminus or 5′ terminus of the nucleotide opposite to the target adenosine. In certain embodiments, the dRNA comprises 2′-O-methylations in the first and last 3 nucleotides, phosphorothiations in the first and last 3 internucleotide linkages, 2′-O-methylations in all uridines, and a 3′-phosphorothiation in the nucleotide opposite to the target adenosine and/or its 5′ and/or 3′ most adjacent nucleotides. In some embodiments, the dRNA comprises 2′-O-methylations in the first and last 5 nucleotides and phosphorothiations in the first and last 5 internucleotide linkages.

In some embodiments, the dRNA does not comprises any modified nucleotides, such as any of the chemically modified nucleotides described herein.

In certain embodiments according to any one of the methods described herein, the efficiency of splicing modulation, exon skipping, or editing on the target RNA is at least about 30%, such as at least about any one of 32%, 35%, 40%, 45%, 50%, 55%, 60%, 65%, 70%, 75%, 80%, 85%, 90% or higher.

In some embodiments, the dRNA is circularized before being introduced to the host cell. In some embodiments, the dRNA is a circRNA formed using a linear RNA in vitro by autocatalysis of a Group I intron comprising a 5′ catalytic Group I intron fragment and a 3′ catalytic Group I intron fragment. In some embodiments, the linear RNA comprises the 3′ catalytic Group I intron fragment flanking the 5′ end of a 3′ exon sequence recognizable by the 3′ catalytic Group I intron fragment, and the 5′ catalytic Group I intron fragment flanking the 3′ end of a 5′ exon sequence recognizable by the 5′ catalytic Group I intron fragment. In some embodiments, the linear RNA further comprises a 5′ homology sequence flanking the 5′ end of the 3′ catalytic Group I intron fragment, and a 3′ homology sequence flanking the 3′ end of the 5′ catalytic Group I intron fragment. In some embodiments, said forming a circRNA comprises: (a) subjecting the linear RNA to a condition that activates autocatalysis of the 5′ catalytic Group I intron fragment and the 3′ catalytic Group I intron fragment to provide a circularized RNA product; and (b) isolating the circularized RNA product, thereby providing the circRNA. Methods of forming circRNA in vitro (including forming circRNA by chemical ligation) are further described in International Patent Applications WO2022150974 and WO2022037692, which are specifically incorporated herein by reference.

### Host Cell

In some embodiments, the host cell is a prokaryotic cell. In some embodiments, the host cell is a eukaryotic cell. Preferably, the host cell is a mammalian cell. Most preferably, the host cell is a human cell. In some embodiments, the host cell is a murine cell. In some embodiments, the host cell is a plant cell or a fungal cell.

In some embodiments, the host cell is a cell line, such as HEK293T, HT29, A549, HepG2, RD, SF268, SW13 and HeLa cell. In some embodiments, the host cell is a primary cell, such as fibroblast, epithelial, or immune cell. In some embodiments, the host cell is a T cell. In some embodiments, the host cell is a post-mitosis cell. In some embodiments, the host cell is a cell of the central nervous system (CNS), such as a brain cell, e.g., a cerebellum cell.

In some embodiments, the ADAR is endogenous to the host cell. In some embodiments, the ADAR is naturally or endogenously present in the host cell, for example, naturally or endogenously present in the eukaryotic cell. In some embodiments, the ADAR is endogenously expressed by the host cell. In certain embodiments, the ADAR is exogenously introduced into the host cell. In some embodiments, the ADAR is ADAR1 and/or ADAR2. In certain embodiments, the ADAR is one or more ADARs selected from the group consisting of hADAR1, hADAR2, mouse ADAR1 and ADAR2. In some embodiments, the ADAR is ADAR1, such as p110 isoform of ADAR1 (“ADAR1^{p110}”>) and/or p150 isoform of ADAR1 (“ADAR1^{p150}”). In some embodiments, the ADAR is ADAR2. In some embodiments, the ADAR is an ADAR2 expressed by the host cell, e.g., ADAR2 expressed by cerebellum cells.

In some embodiments, the ADAR is an ADAR exogenous to the host cell. In some embodiments, the ADAR is a hyperactive mutant of a naturally occurring ADAR. In some embodiments, the ADAR is ADAR1 comprising an E1008Q mutation. In some embodiments, the ADAR is not a fusion protein comprising a binding domain. In some embodiments, the ADAR does not comprise an engineered double-strand nucleic acid-binding domain. In some embodiments, the ADAR does not comprise a MCP domain that binds to MS2 hairpin that is fused to the complementary RNA sequence in the dRNA. In some embodiments, the ADAR does not comprise a DSB.

In some embodiments according to any one of the methods described herein, the host cell has high expression level of ADAR1 (such as ADAR1^{p110} and/or ADAR1^{p150}), e.g., at least about any one of 10%, 20%, 50%, 100%, 2×, 3×, 5×, or more relative to the protein expression level of β-tubulin. In some embodiments, the host cell has high expression level of ADAR2, e.g., at least about any one of 10%, 20%, 50%, 100%, 2×, 3×, 5×, or more relative to the protein expression level of β-tubulin. In some embodiments, the host cell has low expression level of ADAR3, e.g., no more than about any one of 5×, 3×, 2×, 100%, 50%, 20% or less relative to the protein expression level of β-tubulin.

dRNA and circRNA Characteristics

In certain embodiments, the dRNA (or circRNA) comprises at least about any one of 70, 75, 80, 90, 100, 110, 120, 130, 140, 150, 160, 170, 180, 190, 200, 210, 220, 230, 240, or 250 nucleotides. In certain embodiments, the dRNA (or circRNA) comprises no more than about any one of 80, 90, 100, 110, 120, 130, 140, 150, 160, 170, 180, 190, 200, 210, 220, 230, 240, or 250 nucleotides. In certain embodiments, the dRNA (or circRNA) is about any one of 70-220, 70-210, 70-200, 70-190, 70-180, 70-170, 70-160, 70-150, 70-140, 70-130, 70-120, 70-110, 70-100, 70-90, 70-80, 75-200, 80-190, 85-180, 90-170, 95-160, 100-200, 100-150, 100-175, 110-200, 110-175, 110-150, or 105-140 nucleotides in length.

In some embodiments according to any one of the methods or use described herein, the dRNA does not comprise an ADAR-recruiting domain. “ADAR-recruiting domain” can be a nucleotide sequence or structure that binds at high affinity to ADAR, or a nucleotide sequence that binds to a binding partner fused to ADAR in an engineered ADAR construct. Exemplary ADAR-recruiting domains include, but are not limited to, GluR-2, GluR-B (R/G), GluR-B (Q/R), GluR-6 (R/G), 5HT2C, and FlnA (Q/R) domain; see, for example, Wahlstedt, Helene, and Marie, “Site-selective versus promiscuous A-to-I editing.” Wiley Interdisciplinary Reviews: RNA 2.6 (2011): 761-771, which is incorporated herein by reference in its entirety. In some embodiments, the dRNA does not comprise a double-stranded portion. In some embodiments, the dRNA does not comprise a hairpin, such as MS2 stem loop. In some embodiments, the dRNA is single stranded. In some embodiments, the dRNA does not comprise a DSB-binding domain. In some embodiments, the dRNA consists of (or consists essentially of) the complementary RNA sequence.

In some embodiments, the dRNA does comprise an ADAR-recruiting domain. Exemplary ADAR-recruiting domains include, but are not limited to, GluA2-EIE1 (SEQ ID NO: 17), GluA2-EIE2 (SEQ ID NO: 18), Gabra-3-EIE (SEQ ID NO: 19), FLNB-EIE (SEQ ID NO: 20), GluA2 (R/G) (SEQ ID NO: 21), GluK1-EIE1 (SEQ ID NO: 22), GluK1-EIE2 (SEQ ID NO: 23), GluK2-EIE, CopaE-EIE (SEQ ID NO: 24), GluR-2, GluR-B (R/G), GluR-B (Q/R), GluR-6 (R/G), 5HT2C, and FlnA (Q/R) domain; see, for example, Wahlstedt, Helene, and Marie, “Site-selective versus promiscuous A-to-I editing.” Wiley Interdisciplinary Reviews: RNA 2.6 (2011): 761-771, which is incorporated herein by reference in its entirety. Editing inducer elements (EIEs) were recognized by Daniel et al, (2017) as a general mechanism contributing to efficient editing at specific sites. These elements include stem structures, which are separated from the main target stem by a long internal loop. EIEs for several efficiently edited adenosine residues were identified and shown to induce editing independent of their sequence and location upstream or downstream to the edited adenosine. This suggests that the increased editing efficiency probably results from recruiting ADAR enzymes to the RNA molecule. Moreover, the large loop separating the EIE from the edited site stem was shown to contribute to the site selectivity by limiting the editing of adenosine residues adjacent to the specific site. See, for example, Daniel C, Widmark A, Rigardt D, Ohman M. Editing inducer elements increases A-to-I editing efficiency in the mammalian transcriptome. Genome Biol. 2017. Doi: 10.1186/s13059-017-1324-x, and Danan-Gotthold, M., Levanon, E. Y. Promoting RNA editing by ADAR attraction. Genome Biol 18, 196 (2017), each of which is incorporated herein by reference in its entirety. In some embodiments, the dRNA does comprise a double-stranded portion. In some embodiments, the dRNA does comprise a hairpin, such as MS2 stem loop. In some embodiments, the dRNA is single stranded. In some embodiments, the dRNA does comprise a DSB-binding domain. In some embodiments, the dRNA consists of (or consists essentially of) the complementary RNA sequence.

In some embodiments, the dRNA does not comprise chemical modifications. In some embodiments, the dRNA does not comprise a chemically modified nucleotide, such as 2′-O-methyl nucleotide or a nucleotide having a phosphorothioate linkage. In some embodiments, the dRNA is not an antisense oligonucleotide (ASO).

The dRNAs described herein comprise a targeting RNA sequence that is at least partially complementary to the target sequence in the pre-mRNA. In some embodiments, the target RNA sequence comprises one or more splice sites, such as at least one of a 5′ splice site (5′ ss), a polypyrimidine tract, a branchpoint and a 3′ splice site (3′ ss) of a pre-mRNA, and the dRNA molecule is capable of recruiting an ADAR to the target RNA. In certain embodiments, the target RNA sequence comprises at least one, two, three or four of the 5′ splice site (5′ ss), 3′ splice site (3′ ss), the polypyrimidine tract, and the branchpoint. In certain embodiments, the target RNA sequence comprises at least one, two or three of the 3′ splice site (3′ ss), the polypyrimidine tract, and the branchpoint. In certain embodiments, the 3′ ss comprises the nucleotide sequence of YAG, the polypyrimidine tract has a length of 4-25 nucleotides, such as 4, 5, 6, 7, 8, 9, 10, 11, 12, 13, 14, 15, 16, 17, 18, 19, 20, 21, 22, 23, 24 or 25 nucleotides, and the branchpoint comprises the nucleotide sequence of YUNAY, such as CURAY, YNCURAY, YNCURAC or YNYURAY; where Y is independently a pyrimidine, N is any nucleotide, and R is a purine. In certain embodiments, the target RNA sequence comprises, in 5′ to the 3′ direction: the branchpoint comprising the nucleotide sequence of YNYURAY and the polypyrimidine tract having a length of 10-20 nucleotides, the polypyrimidine tract having a length of 10-20 nucleotides and the 3′ ss comprising the nucleotide sequence of YAG; or the branchpoint comprising the nucleotide sequence of YNYURAY, the polypyrimidine tract having a length of 10-20 nucleotides; and the 3′ ss comprising the nucleotide sequence of YAG, optionally, the target RNA sequence further comprises a 5′ splice site (5′ ss). In certain embodiments, the target RNA sequence comprises an exonic splicing enhancer sequence (ESE) of the target exon. An exonic splicing enhancer (ESE) is a DNA sequence motif within an exon that directs, or enhances, accurate splicing of heterogeneous nuclear RNA (hnRNA) or pre-mRNA into messenger RNA (mRNA).

In certain embodiments according to any one of the methods described herein, the targeting RNA sequence in the dRNA comprises at least about any one of 30, 35, 40, 45, 50, 55, 60, 65, 70, 75, 80, 90, 100, 110, 120, 130, 140, 150, 160, 170, 180, 190, 200, 210, 220, 230, 240, or 250 nucleotides. In certain embodiments according to any one of the methods described herein, the targeting RNA sequence in the dRNA comprises no more than about any one of 80, 90, 100, 110, 120, 130, 140, 150, 160, 170, 180, 190, 200, 210, 220, 230, 240, or 250 nucleotides. In certain embodiments, the targeting RNA sequence in the dRNA is about any one of 70-220, 70-210, 70-200, 70-190, 70-180, 70-170, 70-160, 70-150, 70-140, 70-130, 70-120, 70-110, 70-100, 70-90, 70-80, 75-200, 80-190, 85-180, 90-170, 95-160, 100-200, 100-150, 100-175, 110-200, 110-175, 110-150, or 105-140 nucleotides in length. In some embodiments, the targeting RNA sequence in the dRNA is about 71 nucleotides long. In some embodiments, the dRNA is about 111 nucleotides long.

In some embodiments, the targeting RNA sequence comprises a cytidine, adenosine or uridine directly opposite a target adenosine residue in the target sequence of the pre-mRNA, which include, but is not limited to: 1) an adenosine at a 3′ splice site of the 5′ intron comprising an AG splicing sequence; 2) an adenosine at the branch point of the 5′ intron; 3) an adenosine at a branch point sequence comprising YNYURAY, wherein U is C or U, R is A or G, and Y is C or T; and 4) adenosine in the pyrimidine-rich sequence of the 5′ intron. In some embodiments, the targeting RNA sequence comprises a cytidine mismatch directly opposite the target adenosine residue in the target sequence. In some embodiments, the cytidine mismatch is located at least 5 nucleotides, e.g., at least 10, 15, 20, 25, 30, or more nucleotides, away from the 5′ end of the targeting RNA sequence. In some embodiments, the cytidine mismatch is located at least 20 nucleotides, e.g., at least 25, 30, 35, or more nucleotides, away from the 3′ end of the targeting RNA sequence. In some embodiments, the cytidine mismatch is not located within 20 (e.g., 15, 10, 5 or fewer) nucleotides away from the 3′ end of the targeting RNA sequence. In some embodiments, the cytidine mismatch is located at least 20 nucleotides (e.g., at least 25, 30, 35, or more nucleotides) away from the 3′ end and at least 5 nucleotides (e.g., at least 10, 15, 20, 25, 30, or more nucleotides) away from the 5′ end of the targeting RNA sequence. In some embodiments, the cytidine mismatch is located in the center of the targeting RNA sequence. In some embodiments, the cytidine mismatch is located within 20 nucleotides (e.g., 15, 10, 9, 8, 7, 6, 5, 4, 3, 2, or 1 nucleotide) of the center of the targeting sequence in the dRNA.

In some embodiments according to any one of the methods described herein, the targeting RNA sequence further comprises one or more guanosine(s), such as 1, 2, 3, 4, 5, 6, or more Gs, that is each directly opposite a non-target adenosine in the target sequence. In some embodiments, the targeting RNA sequence comprises two or more consecutive mismatch nucleotides (e.g., 2, 3, 4, 5, or more mismatch nucleotides) opposite a non-target adenosine in the target sequence.

In some embodiments, the target sequence comprises no more than about 20 non-target As, such as no more than about any one of 15, 10, 9, 8, 7, 6, 5, 4, 3, 2, or 1 non-target A. The Gs and consecutive mismatch nucleotides opposite non-target As may reduce off-target editing effects by ADAR.

In certain embodiments according to any one of the methods described herein, the 5′ nearest neighbor of the target adenosine residue is a nucleotide selected from U, C, A and G with the preference U>C≈A>G and the 3′ nearest neighbor of the target adenosine residue is a nucleotide selected from G, C, A and U with the preference G>C>A≈U. In certain embodiments, the target adenosine residue is in a two-base motif AG, and the dRNA comprises a C directly opposite the target A, and a C, G or U directly opposite the G in the two-base motif.

In certain embodiments according to any one of the methods described herein, the method further comprises introducing an inhibitor of ADAR3 to the host cell. In some embodiments, the inhibitor of ADAR3 is an RNAi against ADAR3, such as a shRNA against ADAR3 or a siRNA against ADAR3. In some embodiments, the method further comprises introducing a stimulator of interferon to the host cell. In some embodiments, the ADAR is inducible by interferon, for example, the ADAR is ADAR^{p150}. In some embodiments, the stimulator of interferon is IFNα. In some embodiments, the inhibitor of ADAR3 and/or the stimulator of interferon are encoded by the same construct (e.g., vector) that encodes the dRNA.

In certain embodiments according to any one of the methods described herein, the efficiency of splicing modulation or exon skipping in the pre-mRNA is at least about 10%, such as at least about any one of 15%, 20%, 25%, 30%, 35%, 40%, 45%, 50%, 55%, 60%, 65%, 70%, 75%, 80%, 85%, 90%, 95%, or higher. In some embodiments, the efficiency of splicing modulation or exon skipping is determined by Southern blot. In some embodiments, the efficiency of exon skipping is determined by next-generation sequencing.

In certain embodiments, the method has low editing rate at any adenosine residue. In some embodiments, the method has lower than about 1% (e.g., no more than about any one of 0.5%, 0.1%, 0.05%, 0.01%, 0.001% or lower) editing efficiency on As in the target sequence. In some embodiments, the method does not edit As in the target sequence. In some embodiments, the method has lower than about 0.1% (e.g., no more than about any one of 0.05%, 0.01%, 0.005%, 0.001%, 0.0001% or lower) editing efficiency on As in the target sequence.

In some embodiments, the method has low off-target editing rate. In some embodiments, the method has lower than about 1% (e.g., no more than about any one of 0.5%, 0.1%, 0.05%, 0.01%, 0.001% or lower) editing efficiency on non-target As in the target RNA. In some embodiments, the method does not edit non-target As in the target RNA. In some embodiments, the method has lower than about 0.1% (e.g., no more than about any one of 0.05%, 0.01%, 0.005%, 0.001%, 0.0001% or lower) editing efficiency on As in non-target RNA.

In certain embodiments according to any one of the methods described herein, the method does not induce immune response, such as innate immune response. In some embodiments, the method does not induce interferon and/or interleukin expression in the host cell. In some embodiments, the method does not induce IFN-β and/or IL-6 expression in the host cell.

Also provided are exon-skipped mRNAs or host cells having an exon-skipped mRNAs produced by any one of the methods described herein.

Methods of Delivery and Construct Encoding the dRNA

Methods of non-viral delivery of nucleic acids include lipofection, nucleofection, microinjection, biolistics, virosomes, liposomes, immunoliposomes, polycation or lipid: nucleic acid conjugates, electroporation, nanoparticles, exosomes, microvesicles, or gene-gun, naked DNA and artificial virions.

The use of RNA or DNA viral based systems for the delivery of nucleic acids has high efficiency in targeting a virus to specific cells and trafficking the viral payload to the cellular nuclei.

In certain embodiments according to any one of the methods described herein, the method comprises introducing a viral vector (such as an AAV or a lentiviral vector) encoding the dRNA to the host cell. In some embodiments, the vector is a recombinant adeno-associated virus (rAAV) vector. In some embodiments, the construct is flanked by one or more AAV inverted terminal repeat (ITR) sequences. In some embodiments, the construct is flanked by two AAV ITRs. In some embodiments, the AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV serotype ITRs. In some embodiments, the AAV ITRs are AAV2 ITRs. In some embodiments, the vector further comprises a stuffer nucleic acid. In some embodiments, the stuffer nucleic acid is located upstream or downstream of the nucleic acid encoding the dRNA. In some embodiments, the vector is a self-complementary rAAV vector. In some embodiments, the vector comprises first nucleic acid sequence encoding the dRNA and a second nucleic acid sequence encoding a complement of the dRNA, wherein the first nucleic acid sequence can form intrastrand base pairs with the second nucleic acid sequence along most or all of its length. In some embodiments, the first nucleic acid sequence and the second nucleic acid sequence are linked by a mutated AAV ITR, wherein the mutated AAV ITR comprises a deletion of the D region and comprises a mutation of the terminal resolution sequence. In some embodiments, the vector is encapsidated in a rAAV particle. In some embodiments, the AAV viral particle comprises an AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV2/2-7m8, AAV DJ, AAV2 N587A, AAV2 E548A, AAV2 N708A, AAV2 V708K, AAV2-HBKO, AAVDJ8, AAV-PHP.B, AAV-PHP.eB, AAV-BR1, AAVHSC15, AAVHSC17, goat AAV, AAV1/AAV2 chimeric, bovine AAV, mouse AAV, or rAAV2/HboV1 serotype capsid.

In some embodiments, the method comprises introducing a plasmid encoding the dRNA to the host cell. In some embodiments, the method comprises electroporation of the dRNA (e.g., synthetic dRNA) into the host cell. In some embodiments, the method comprises transfection of the dRNA into the host cell.

The presence or absence of exon skipping can be determined by analyzing the mature mRNA. An exemplary suitable method for analyzing the mature mRNA is RT-PCR and/or sequencing, using methods that are well-known to the person skilled in the art.

### Pre-mRNA and Target Exons

The pre-mRNA described herein can be transcribed from any gene of interest. In some embodiments, the pre-mRNA is from a gene selected from the group consisting of dystrophin, RELA, Trp53, PPIB, STAT3, SMN1, CFTR, beta-globin, Factor VIII, Factor IX, TP53, mesenchymal epithelial transition factor receptor (MET), KRAS, RAB7A, ABCA4, SERPINA1, HEXA, LRRK2, SNCA, DMD, APP, MAPT, CFTR, ALAS1, ATP7B, HFE, LIP A, CEP290, USH2A, CD274, HTT, COL7A1, MS4A, SGCG, ATXN3, NOTCH3, COL4A5, NF1, PCSK9 start site, SCNN1A, a collagen VI alpha 1 gene (COL6A1), a myotubular myopathy 1 gene (MTM1), a dysferlin gene (DYSF), a laminin-alpha 2 gene (LAMA2), an emery-dreyfuss muscular dystrophy gene (EMD), a calpain 3 gene (CAPN3), and superoxide dismutase 1 (SOD1).

For example, in some embodiments, the pre-mRNA is from a dystrophin gene, and the target exon is selected from the group consisting of exons 3, 4, 5, 8, 10-16, 19-40, 42-44, 46, 47, and 50-53. In some embodiments, the pre-mRNA is from a RELA gene and the target exon is exon 7. In some embodiments, the pre-mRNA is from a Trp53 genes and the target exon is selected from the group consisting of exons 2 and 5. In some embodiments, the pre-mRNA is from a PPIB gene and the target exon is exon 3. In some embodiments, the pre-mRNA is from an SMN1 gene and the target exon is exon 7. In some embodiments, the pre-mRNA is from a CFTR gene, and the target exon is selected from the group consisting of exons 9, 10, 11, 12, 13, and 23. In some embodiments, the pre-mRNA is from a beta-globin gene and the target exon is selected from the group consisting of exons 2 and 3. In some embodiments, the pre-mRNA is from a Factor VIII gene, and the target exon is selected from the group consisting of exon 5, 6, 7, and 8. In some embodiments, the pre-mRNA is from a Factor IX gene, and wherein the target exon is exon 2. In some embodiments, the pre-mRNA is from a SOD1 gene, and wherein the target exon is exon 2.

### III. System Comprising DRNA or Construct Encoding the DRNA

Also provided herein are systems comprising dRNAs or constructs useful for any one of the methods described herein. Modulating splicing of pre-mRNAs using specifically designed RNAs, namely, deaminase-recruiting RNAs (“dRNAs”) which are either circular (circRNA) or linear dRNA, optionally a linear dRNA that can form circRNA within a cell. The dRNAs contain a targeting RNA sequence that is at least partially complementary to a portion of a target exon and/or a portion of its flanking intron and recruits an ADAR to the target exon and/or intron, thereby modulating the splicing of the pre-mRNA. In some embodiments, the modulation of the pre-mRNA splicing allows the pre-mRNA to be spliced to form a mature mRNA not including the target exon (“exon skipping”). In some embodiments, the recruitment of the ADAR blocks access of one or more splicing factors to a splicing site for the target exon, thereby modulating the splicing of the target exon. This can be accomplished, for example, without editing of the pre-mRNA. In some embodiments, on the other hand, the recruitment of the ADAR results in editing of the pre-mRNA to change sites which are critical for splicing, further reducing splicing efficiency of the target exon. Surprisingly, the splicing modulation is much more effective when the dRNA is circRNA or forms circRNA within a cell. It was also observed that the splicing modulation is much more effective above certain threshold length of the targeting RNA sequence.

The present application thus in some embodiments provides a system for modulating splicing of a target exon in a pre-mRNA in a host cell, the pre-mRNA comprising the target exon flanked by a 5′ intron and a 3′ intron, wherein the system comprises a deaminase-recruiting RNA (dRNA) or a construct comprising a nucleic acid encoding the dRNA, wherein: (1) dRNA comprises a targeting RNA sequence that is at least partially complementary to a target sequence in the pre-mRNA comprising a portion of the target exon, a portion of the 5′ intron, and/or a portion of the 3′ intron; and (2) the dRNA is capable of recruiting an ADAR to the target exon, the 5′ intron, and/or the 3′ intron.

In some embodiments, there is provided a system for inducing exon skipping in a pre-mRNA in a host cell, comprising a dRNA or construct comprising a nucleic acid encoding the dRNA, wherein the dRNA is circRNA, a linear dRNA, or a linear dRNA that can forms a circRNA within a host cell, and wherein the dRNA comprises a targeting RNA sequence that is at least partially complementary to a portion of the target exon and/or a portion of the 5′ intron (or 3′ intron) and recruits an ADAR to the target exon and/or 5′ intron (or 3′ intron).

In some embodiments, there is provided a system for inducing exon skipping in a pre-mRNA in a host cell, the pre-mRNA comprising a target exon flanked by a 5′ intron and a 3′ intron, wherein the system comprises a deaminase-recruiting RNA (dRNA) or a construct comprising a nucleic acid encoding the dRNA into the host cell, wherein: (1) the dRNA is a circRNA, a linear dRNA, or a linear dRNA capable of forming a circRNA within a host cell; (2) the dRNA comprises a targeting RNA sequence that is at least partially complementary to a target sequence in the pre-mRNA comprising a portion of the target exon and/or a portion of the 5′ intron; and (3) the dRNA or circRNA is capable of recruiting an ADAR to the target exon and/or 5′ intron.

Any one of the dRNAs or constructs described in this section may be used in the systems and methods of splicing modulation and treatment described herein. It is intended that any of the features and parameters described herein for dRNAs or constructs can be combined with each other, as if each and every combination is individually described. The dRNAs described herein do not comprise a tracrRNA, crRNA or gRNA used in a CRISPR/Cas system. In some embodiments, there is provided a deaminase-recruiting RNA (dRNA) for deamination of a target adenosine in a target RNA by recruiting an ADAR, comprising a complementary RNA sequence that hybridizes to the target RNA.

In one aspect, the present provides a construct comprising any one of the deaminase-recruiting RNAs described herein. In certain embodiments, the construct is a viral vector (preferably a lentivirus vector) or a plasmid. In some embodiments, the construct encodes a single dRNA. In some embodiments, the construct encodes a plurality (e.g., about any one of 1, 2, 3, 4, 5, 10, 20 or more) dRNAs.

In one aspect, the present application provides a library comprising a plurality of the dRNAs or a plurality of the constructs described herein.

In one aspect, the present application provides a system or a host cell comprising the dRNA or the construct described herein. In certain embodiments, the host cell is a prokaryotic cell or a eukaryotic cell. Preferably, the host cell is a mammalian cell. Most preferably, the host cell is a human cell.

Circular dRNAs and Constructs

In some embodiments, the dRNA is a linear RNA that is capable of forming a circRNA. In some embodiments, the dRNA is circulated by the Tornado method. In some embodiments, the dRNA transcript is also flanked a 5′ and/or 3′ litigation sequences which are then optionally flanked by the 5′-Twister ribozyme and/or 3′-Twister ribozymes, respectively. In some embodiments, the 3′ ligation sequence and the 5′ ligation sequence are at least partially complementary to each other. In some embodiments, the 3′ ligation sequence and the 5′ ligation sequence are at least about 50%, at least about 55%, at least about 60%, at least about 65%, at least about 70%, at least about 75%, at least about 80%, at least about 85%, at least about 90%, at least about 95%, at least about 96%, at least about 97%, at least about 98% or at least about 99% complementary to each other. In some embodiments, the 3′ ligation sequence and the 5′ ligation sequence are fully complementary to each other.

In some embodiments, the 3′ ligation sequence and the 5′ ligation sequence are independently at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides or at least about 100 nucleotides in length. In some embodiments, the 3′ ligation sequence and the 5′ ligation sequence are independently about 20-30 nucleotides, about 30-40 nucleotides, about 40-50 nucleotides, about 50-60 nucleotides, about 60-70 nucleotides, about 70-80 nucleotides, about 80-90 nucleotides, about 90-100 nucleotides, about 100-125 nucleotides, about 125-150 nucleotides, about 20-50 nucleotides, about 50-100 nucleotides or about 100-150 nucleotides in length.

In some embodiments, the dRNA is circularized by an RNA ligase. In some embodiments, the RNA ligase is expressly endogenously in the host cell. In some embodiments, the RNA ligase is RNA ligase RtcB. In some embodiments, the RNA ligase RtcB is expressly endogenously in the host cell. In some embodiments, the dRNA is circularized through in vitro enzymatic ligation (e.g., using RNA or DNA ligase) or chemical ligation (e.g., using cyanogen bromide or a similar condensing agent).

Also provided herein is a construct comprising a nucleic acid encoding the dRNA. In some embodiments, the dRNA is introduced by a construct comprising a nucleic acid encoding the dRNA. In some embodiments, the construct further comprises a 3′ twister ribozyme sequence linked to the 3′ end of the nucleic acid encoding the dRNA and a 5′ twister ribozyme sequence linked to the 5′ end of the nucleic acid encoding the dRNA. In some embodiments, the 3′ twister sequence is twister P3 U2A and the 5′ twister sequence is twister P1. In some embodiments, wherein the 5′ twister sequence is twister P3 U2A and the 3′ twister sequence is twister P1. In some embodiments, the dRNA undergoes autocatalytic cleavage. In some embodiments, the catalyzed dRNA product comprises a 5′-hydroxyl group and a 2′,3′-cyclic phosphate at the 3′ terminus. In some embodiments, the catalyzed dRNA product is ligated by ubiquitous endogenous RNA ligase (e.g., RNA ligase RtcB). In some embodiments, the construct is a plasmid or a viral vector.

In some embodiments according to any one of the systems, dRNAs, constructs, libraries or compositions described herein, the targeting RNA sequence comprises a cytidine, adenosine or uridine directly opposite a target adenosine to be edited in the target sequence. In some embodiments, the targeting RNA sequence further comprises one or more guanosine(s) that is each directly opposite a non-target adenosine in the target sequence. In certain embodiments, the 5′ nearest neighbor of the target adenosine residue is a nucleotide selected from U, C, A and G with the preference U>C≈A>G and the 3′ nearest neighbor of the target adenosine residue is a nucleotide selected from G, C, A and U with the preference G>C>A≈U. In some embodiments, the 5′ nearest neighbor of the target adenosine residue is U. In some embodiments, the 5′ nearest neighbor of the target adenosine residue is C or A. In some embodiments, the 3′ nearest neighbor of the target adenosine residue is G. In some embodiments, the 3′ nearest neighbor of the target adenosine residue is C.

In some embodiments according to any one of the systems, dRNAs, constructs, libraries or compositions described herein, the target adenosine residue is in a two-base motif AG in the target sequence. In some embodiments, the dRNA comprises C directly opposite the target A, and a C, G or U directly opposite the G in the two-base motif.

In some embodiments, the dRNA comprises a cytidine mismatch directly opposite a target adenosine residue in the target sequence. In some embodiments, the cytidine mismatch is close to the center of the targeting RNA sequence, such as within 20, 15, 10, 5, 4, 3, 2, or 1 nucleotide away from the center of the targeting RNA sequence. In some embodiments, the cytidine mismatch is at least 5 nucleotides away from the 5′ end of the targeting RNA sequence. In some embodiments, the cytidine mismatch is at least 20 nucleotides away from the 3′ end of the targeting RNA sequence.

In some embodiments according to any one of the systems, dRNAs, constructs, libraries or compositions described herein, the dRNA comprises more than about any one of 70, 75, 80, 90, 100, 110, 120, 130, 140, 150, 160, 170, 180, 190, 200, 210, 220, 230, 240, or 250 nucleotides. In certain embodiments, the dRNA is about any one of 70-210, 70-200, 70-190, 70-180, 70-170, 70-160, 70-150, 70-140, 70-130, 70-120, 70-110, 70-100, 70-90, 70-80, 75-200, 80-190, 85-180, 90-170, 95-160, 100-150 or 105-140 nucleotides in length.

The dRNA of the present application comprises a targeting RNA sequence that hybridizes to the target RNA. The targeting RNA sequence is perfectly complementary or substantially complementarity to the target sequence to allow hybridization of the targeting RNA sequence to the target sequence. In some embodiments, the targeting RNA sequence has 100% sequence complementarity as the target sequence. In some embodiments, the targeting RNA sequence is at least about any one of 70%, 80%, 85%, 90%, 95%, 96%, 97%, 98%, or 99% or more complementary to over a continuous stretch of at least about any one of 20, 40, 60, 80, 100, 150, 200, or more nucleotides in the target sequence. In some embodiments, the dsRNA formed by hybridization between the targeting RNA sequence and the target sequence has one or more (e.g., 1, 2, 3, 4, 5, 6, 7, 8, 9, 10 or more) non-Watson-Crick base pairs (i.e., mismatches).

ADAR, for example, human ADAR enzymes edit double stranded RNA (dsRNA) structures with varying specificity, depending on a number of factors. One important factor is the degree of complementarity of the two strands making up the dsRNA sequence. Perfect complementarity of between the dRNA and the target RNA usually causes the catalytic domain of ADAR to deaminate adenosines in a non-discriminative manner. The specificity and efficiency of ADAR can be modified by introducing mismatches in the dsRNA region. For example, A-C mismatch is preferably recommended to increase the specificity and efficiency of deamination of the adenosine to be edited. Conversely, at the other A (adenosine) positions than the target adenosine residue (i.e., “non-target A”), the G-A mismatch can reduce off-target editing. Perfect complementarity is not necessarily required for a dsRNA formation between the dRNA and its target RNA, provided there is substantial complementarity for hybridization and formation of the dsRNA between the dRNA and the target RNA. In some embodiments, the dRNA sequence or single-stranded RNA region thereof has at least about any one of 70%, 80%, 85%, 90%, 95%, 96%, 97%, 98%, or 99% of sequence complementarity to the target RNA, when optimally aligned. Optimal alignment may be determined with the use of any suitable algorithm for aligning sequences, non-limiting examples of which include the Smith-Waterman algorithm, the Needleman-Wimsch algorithm, algorithms based on the Burrows-Wheeler Transform (e.g., the Burrows Wheeler Aligner).

The nucleotides neighboring the target adenosine also affect the specificity and efficiency of deamination. For example, the 5′ nearest neighbor of the target adenosine to be edited in the target RNA sequence has the preference U>C≈A>G and the 3′ nearest neighbor of the target adenosine to be edited in the target RNA sequence has the preference G>C>A≈U in terms of specificity and efficiency of deamination of adenosine.

Besides the target adenosines, there may be one or more adenosines in the target RNA which are not desirable to be edited. With respect to these adenosines, it is preferable to reduce their editing efficiency as much as possible. It is found that where guanosine is directly opposite an adenosine in the target RNA, the deamination efficiency is significantly decreased. Therefore, in order to decrease off-target deamination, dRNAs can be designed to comprise one or more guanosines directly opposite one or more adenosine(s) other than the target adenosine to be edited in the target RNA.

The desired level of specificity and efficiency of editing the target RNA sequence may depend on different applications. Following the instructions in the present patent application, those of skill in the art will be capable of designing a dRNA having complementary or substantially complementary sequence to the target RNA sequence according to their needs, and, with some trial and error, obtain their desired results. As used herein, the term “mismatch” refers to opposing nucleotides in a double stranded RNA (dsRNA) which do not form perfect base pairs according to the Watson-Crick base pairing rules. Mismatch base pairs include, for example, G-A, C-A, U-C, A-A, G-G, C-C, U-U base pairs. Taking A-C match as an example, where a target adenosine residue is to be edited in the target RNA, a dRNA is designed to comprise a C opposite the A to be edited, generating a A-C mismatch in the dsRNA formed by hybridization between the target RNA and dRNA.

In some embodiments, the dsRNA formed by hybridization between the dRNA and the target RNA does not comprise a mismatch. In some embodiments, the dsRNA formed by hybridization between the dRNA and the target RNA comprises one or more, such as any one of 1, 2, 3, 4, 5, 6, 7 or more mismatches (e.g., the same type of different types of mismatches). In some embodiments, the dsRNA formed by hybridization between the dRNA and the target RNA comprises one or more kinds of mismatches, for example, 1, 2, 3, 4, 5, 6, 7 kinds of mismatches selected from the group consisting of G-A, C-A, U-C, A-A, G-G, C-C and U-U.

The mismatch nucleotides in the dsRNA formed by hybridization between the dRNA and the target RNA can form bulges which can promote the efficiency of editing of the target RNA. There may be one (which is only formed at the target adenosine) or more bulges formed by the mismatches. The additional bulge-inducing mismatches may be upstream and/or downstream of the target adenosine. The bulges may be single-mismatch bulges (caused by one mismatching base pair) or multi-mismatch bulges (caused by more than one consecutive mismatching base pairs, preferably two or three consecutive mismatching base pairs).

The targeting RNA sequence in the dRNA is single-stranded. The dRNA may be entirely single-stranded or have one or more (e.g., 1, 2, 3, or more) double-stranded regions and/or one or more stem loop regions. In some embodiments, the targeting RNA sequence is at least about any one of 30, 35, 40, 45, 50, 55, 60, 65, 70, 75, 80, 90, 100, 110, 120, 130, 140, 150, 160, 170, 180, 190, 200 or more nucleotides. In certain embodiments, the targeting RNA sequence is about any one of 30-220, 40-220, 50-220, 60-220, 70-220, 70-210, 70-200, 70-190, 70-180, 70-170, 70-160, 70-150, 70-140, 70-130, 70-120, 70-110, 70-100, 70-90, 70-80, 75-200, 80-190, 85-180, 90-170, 95-160, 100-200, 100-150, 100-175, 110-200, 110-175, 110-150, or 105-140 nucleotides in length. In some embodiments, the targeting RNA sequence is about 71 nucleotides long. In some embodiments, the targeting RNA sequence is about 111 nucleotides long. In some embodiments, the targeting RNA sequence is about 151 nucleotides long.

In some embodiments, the dRNA, apart from the targeting RNA sequence, further comprises regions for stabilizing the dRNA, for example, one or more double-stranded regions and/or stem loop regions. In some embodiments, the double-stranded region or stem loop region of the dRNA comprises no more than about any one of 200, 150, 100, 50, 40, 30, 20, 10 or fewer base-pairs. In some embodiments, the dRNA does not comprise a stem loop or double-stranded region. In some embodiments, the dRNA comprises an ADAR-recruiting domain. In some embodiments, the dRNA does not comprise an ADAR-recruiting domain.

The dRNA may comprise one or more modifications. In some embodiments, the dRNA has one or more modified nucleotides, including nucleobase modification and/or backbone modification. Exemplary modifications to the RNA include, but are not limited to, phosphorothioate backbone modification, 2′-substitutions in the ribose (such as 2′-O-methyl and 2′-fluoro substitutions), LNA, and L-RNA. In some embodiments, the dRNA does not have modifications to the nucleobase or backbone.

The present application also contemplates a construct comprising the dRNA described herein, including, but not limited to, any of the constructs described in the sections above. The term “construct” as used herein refers to DNA or RNA molecules that comprise a coding nucleotide sequence that can be transcribed into RNAs or expressed into proteins. In some embodiments, the construct contains one or more regulatory elements operably linked to the nucleotide sequence encoding the RNA or protein. When the construct is introduced into a host cell, under suitable conditions, the coding nucleotide sequence in the construct can be transcribed or expressed.

In some embodiments, the construct comprises a promoter that is operably linked to the coding nucleotide sequence, such that the promoter controls the transcription or expression of the coding nucleotide sequence. A promoter may be positioned 5′ (upstream) of a coding nucleotide sequence under its control. The distance between the promoter and the coding sequence may be approximately the same as the distance between that promoter and the gene it controls in the gene from which the promoter is derived. As is known in the art, variation in this distance may be accommodated without loss of promoter function. In some embodiments, the construct comprises a 5′ UTR and/or a 3′UTR that regulates the transcription or expression of the coding nucleotide sequence. In some embodiments, the promoter is a Pol III promoter (such as U6 promoter). In some embodiments, the promoter is a Pol II promoter (such as a CMV promoter or a U7 promoter).

In some embodiments, the construct is a vector encoding any one of the dRNAs disclosed in the present application. The term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the transcription or expression of coding nucleotide sequences to which they are operatively linked. Such vectors are referred to herein as “expression vectors”.

Recombinant expression vectors can comprise a nucleic acid of the invention in a form suitable for transcription or expression of the nucleic acid in a host cell. In some embodiments, the recombinant expression vector includes one or more regulatory elements, which may be selected on the basis of the host cells to be used for transcription or expression, which is operatively linked to the nucleic acid sequence to be transcribed or expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

In some embodiments, the vector is a rAAV vector. In some embodiments, the rAAV vector is a vector derived from an AAV serotype, including without limitation, AAV ITRs are AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV capsid serotype or the like. In some embodiments, the nucleic acid in the AAV comprises an ITR of AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV DJ, a goat AAV, bovine AAV, or mouse AAV capsid serotype or the like. In some embodiments, the nucleic acid in the AAV further encodes a dRNA as described herein. Use of any AAV serotype is considered within the scope of the present disclosure. In some embodiments, the vector is encapsidated in a rAAV particle. In some embodiments, the AAV viral particle comprises an AAV1, AAV2, AAV3, AAV4, AAV5, AAV6, AAV7, AAV8, AAVrh8, AAVrh8R, AAV9, AAV10, AAVrh10, AAV11, AAV12, AAV2R471A, AAV2/2-7m8, AAV DJ, AAV2 N587A, AAV2 E548A, AAV2 N708A, AAV2 V708K, AAV2-HBKO, AAVDJ8, AAV-PHP.B, AAV-PHP.eB, AAV-BR1, AAVHSC15, AAVHSC17, goat AAV, AAV1/AAV2 chimeric, bovine AAV, mouse AAV, or rAAV2/HboV1 serotype capsid.

In some embodiments, there is provided a construct (e.g., vector, such as viral vector) comprising a nucleotide sequence encoding the dRNA. In some embodiments, there is provided a construct (e.g., vector, such as viral vector) comprising a nucleotide sequence encoding the ADAR. In some embodiments, there is provided a construct comprising a first nucleotide sequence encoding the dRNA and a second nucleotide sequence encoding the ADAR. In some embodiments, the first nucleotide sequence and the second nucleotide sequence are operably linked to the same promoter. In some embodiments, the first nucleotide sequence and the second nucleotide sequence are operably linked to different promoters. In some embodiments, the promoter is inducible. In some embodiments, the construct does not encode for the ADAR. In some embodiments, the vector further comprises nucleic acid sequence(s) encoding an inhibitor of ADAR3 (e.g., ADAR3 shRNA or siRNA) and/or a stimulator of interferon (e.g., IFN-«).

### IV. Methods of Treatment

The splicing modulation/exon skipping methods and compositions described herein may be used to treat or prevent a disease or condition in an individual.

In some embodiments, the pre-mRNA subject to splicing modulation/exon skipping is associated with a disease or condition of the individual. In some embodiments, the disease or condition is a hereditary genetic disease or a disease or condition associated with one or more acquired genetic mutations.

In some embodiments, there is provided a method of treating or preventing a disease or condition in an individual (e.g., human individual), comprising inducing exon skipping (or modulating splicing) in a pre-mRNA of a host cell in the individual using any one of the methods of exon skipping described herein.

Diseases and conditions suitable for treatment using the methods of the present application include diseases associated with mutations in a target exon. These include, but are not limited to, DMD, SMA, CF, beta-thalassemia, familial amyotrophic lateral sclerosis, Hutchinson-Gilford progeria syndrome (HGPS), Ataxia telangiectasia (ATM), dysferlinopathies, frontotemporal dementia, and hemophilia. For reviews of conditions or diseases that can be treated by a method of the invention, see, e.g., Bauman et al. (2011) Bioeng. Bugs. 2, 125-8, Hammond et al. (2011) Trends Genet. 27, 196-205, Wood et al. (2010) Brain 133, 957-72 or Sazani et al., “Splice-switching oligonucleotides as potential therapeutics” (2007) in Antisense Drug Technology: Principles, Strategies, and Applications, Second Edition (Ed. S. T. Crooke) 89-114 (CRC Press, Boca Raton).

In some embodiments, there is provided a method of treating or preventing Duchenne muscular dystrophy (DMD) in an individual, comprising inducing exon skipping in the pre-mRNA of a dystrophin gene according to the method described herein. In some embodiments, the method comprises administering to the individual an effective amount of a deaminase-recruiting RNA (dRNA) or a construct comprising a nucleic acid encoding the dRNA into the host cell, wherein: (1) the dRNA is a circRNA or forms a circRNA within the host cell; (2) the dRNA comprises a targeting RNA sequence that is at least partially complementary to a target sequence in a dystrophin pre-mRNA comprising a portion of the target exon and/or a portion of the 5′ intron; and (3) the circRNA recruits an ADAR to the target exon and/or 5′ intron; and wherein the pre-mRNA is spliced in the host cell to form a mature mRNA not including the target exon. In some embodiments, the target exon to be skipped is selected from the group consisting of exons 3, 4, 5, 8, 10-16, 19-40, 42-44, 46, 47, and 50-53. In some embodiments, the dRNA comprises the nucleic acid sequence of SEQ ID NOs: 7-9.

In some embodiments, there is provided a method of treating or preventing Spinal muscular atrophy (SMA) in an individual, comprising inducing exon skipping in the pre-mRNA of an SMN1 gene according to the method described herein. In some embodiments, the method comprises administering to the individual an effective amount of a deaminase-recruiting RNA (dRNA) or a construct comprising a nucleic acid encoding the dRNA into the host cell, wherein: (1) the dRNA is a circRNA or forms a circRNA within the host cell; (2) the dRNA comprises a targeting RNA sequence that is at least partially complementary to a target sequence in an SMN1 pre-mRNA comprising a portion of the target exon and/or a portion of the 5′ intron; and (3) the circRNA recruits an ADAR to the target exon and/or 5′ intron; and wherein the pre-mRNA is spliced in the host cell to form a mature mRNA not including the target exon. In some embodiments, the target exon to be skipped is exon 7.

In some embodiments, there is provided a method of treating or preventing Cystic Fibrosis (CF) in an individual, comprising inducing exon skipping in the pre-mRNA of a CFTR gene according to the method described herein. In some embodiments, the method comprises administering to the individual an effective amount of a deaminase-recruiting RNA (dRNA) or a construct comprising a nucleic acid encoding the dRNA into the host cell, wherein: (1) the dRNA is a circRNA or forms a circRNA within the host cell; (2) the dRNA comprises a targeting RNA sequence that is at least partially complementary to a target sequence in a CFTR pre-mRNA comprising a portion of the target exon and/or a portion of the 5′ intron; and (3) the circRNA recruits an ADAR to the target exon and/or 5′ intron; and wherein the pre-mRNA is spliced in the host cell to form a mature mRNA not including the target exon. In some embodiments, the target exon to be skipped is selected from the group consisting of exons 9, 10, 11, 12, 13, and 23.

In some embodiments, there is provided a method of treating or preventing beta-thalassemia in an individual, comprising inducing exon skipping in the pre-mRNA of a beta-globin gene according to the method described herein. In some embodiments, the method comprises administering to the individual an effective amount of a deaminase-recruiting RNA (dRNA) or a construct comprising a nucleic acid encoding the dRNA into the host cell, wherein: (1) the dRNA is a circRNA or forms a circRNA within the host cell; (2) the dRNA comprises a targeting RNA sequence that is at least partially complementary to a target sequence in a beta-globin pre-mRNA comprising a portion of the target exon and/or a portion of the 5′ intron; and (3) the circRNA recruits an ADAR to the target exon and/or 5′ intron; and wherein the pre-mRNA is spliced in the host cell to form a mature mRNA not including the target exon. In some embodiments, the target exon to be skipped is selected from the group consisting of exons 2 and 3.

In some embodiments, there is provided a method of treating or preventing hemophilia in an individual, comprising inducing exon skipping in the pre-mRNA of a Factor VIII gene according to the method described herein. In some embodiments, the method comprises administering to the individual an effective amount of a deaminase-recruiting RNA (dRNA) or a construct comprising a nucleic acid encoding the dRNA into the host cell, wherein: (1) the dRNA is a circRNA or forms a circRNA within the host cell; (2) the dRNA comprises a targeting RNA sequence that is at least partially complementary to a target sequence in a Factor VIII pre-mRNA comprising a portion of the target exon and/or a portion of the 5′ intron; and (3) the circRNA recruits an ADAR to the target exon and/or 5′ intron; and wherein the pre-mRNA is spliced in the host cell to form a mature mRNA not including the target exon. In some embodiments, the target exon to be skipped is selected from the group consisting of exons 5, 6, 7, and 8.

In some embodiments, there is provided a method of treating or preventing hemophilia in an individual, comprising inducing exon skipping in the pre-mRNA of a Factor IX gene according to the method described herein. In some embodiments, the method comprises administering to the individual an effective amount of a deaminase-recruiting RNA (dRNA) or a construct comprising a nucleic acid encoding the dRNA into the host cell, wherein: (1) the dRNA is a circRNA or forms a circRNA within the host cell; (2) the dRNA comprises a targeting RNA sequence that is at least partially complementary to a target sequence in a Factor IX pre-mRNA comprising a portion of the target exon and/or a portion of the 5′ intron; and (3) the circRNA recruits an ADAR to the target exon and/or 5′ intron; and wherein the pre-mRNA is spliced in the host cell to form a mature mRNA not including the target exon. In some embodiments, the target exon to be skipped is exon 2.

In some embodiments, the pre-mRNA is from a mutant TP53 gene, and the disease or condition is Li-Fraumeni syndrome. In some embodiments, the pre-mRNA is from a mutant MET gene, and the disease or condition is hereditary papillary renal carcinoma (HPRC). In some embodiments, the pre-mRNA is from a mutant KRAS gene, and the disease or condition is lung cancer. In some embodiments, the pre-mRNA is from a mutant STAT3 gene, and the disease or condition is hyper-immunoglobulin E syndrome. In some embodiments the pre-mRNA is from a mutant RAB7A gene, and the disease or condition is hereditary sensory neuropathy type 1C. In some embodiments the pre-mRNA is from a mutant ABCA4 gene, and the disease or condition is Stargardt disease or Stickler's Syndrome. In some embodiments the pre-mRNA is from a mutant SERPINA1 gene, and the disease or condition is alpha-1-antitrypsin (A1AT) deficiency. In some embodiments the pre-mRNA is from a mutant HEXA gene, and the disease or condition is Tay-Sachs disease. In some embodiments the pre-mRNA is from a mutant LRRK2 gene, and the disease or condition is Parkinson's disease. In some embodiments the pre-mRNA is from a mutant SNCA gene, and the disease or condition is Parkinson's disease. In some embodiments the pre-mRNA is from a mutant APP gene, and the disease or condition is early-onset Alzheimer disease. In some embodiments the pre-mRNA is from a mutant MAPT gene, and the disease or condition is frontotemporal dementia with parkinsonism-17 (FTDP-17). In some embodiments the pre-mRNA is from a mutant ALAS1 gene, and the disease or condition is anemia or sideroblastic anemia. In some embodiments the pre-mRNA is from a mutant ATP7B gene, and the disease or condition is Wilson disease. In some embodiments the pre-mRNA is from a mutant HFE gene, and the disease or condition is hemochromatosis. In some embodiments the pre-mRNA is from a mutant LIPA gene, and the disease or condition is lysosomal acid lipase deficiency. In some embodiments the pre-mRNA is from a mutant SOD1 gene, and the disease or condition is amyotrophic lateral sclerosis (ALS). In some embodiments the pre-mRNA is from a mutant CEP290 gene, and the disease or condition is ciliopathies. In some embodiments the pre-mRNA is from a mutant USH2A gene, and the disease or condition is Usher syndrome type II and retinitis pigmentosa type 39. In some embodiments the pre-mRNA is from a mutant CD274 gene, and the disease or condition is cancer. In some embodiments the pre-mRNA is from a mutant HTT gene, and the disease or condition is Huntington disease. In some embodiments the pre-mRNA is from a mutant COL7A1 gene, and the disease or condition is dystrophic epidermolysis bullosa. In some embodiments the pre-mRNA is from a mutant MS4A gene, and the disease or condition is Alzheimer's disease. In some embodiments the pre-mRNA is from a mutant SGCG gene, and the disease or condition is limb-girdle muscular dystrophy. In some embodiments the pre-mRNA is from a mutant DYSF gene, and the disease or condition is limb-girdle muscular dystrophy or Miyoshi myopathy. In some embodiments the pre-mRNA is from a mutant COL6A1 gene, and the disease or condition is Collagen VI-related dystrophy. In some embodiments the pre-mRNA is from a mutant MTM1 gene, and the disease or condition is X-linked myotubular myopathy. In some embodiments the pre-mRNA is from a mutant LAMA2 gene, and the disease or condition is LAMA2-related muscular dystrophy. In some embodiments the pre-mRNA is from a mutant EMD gene, and the disease or condition is Emery-Dreifuss muscular dystrophy. In some embodiments the pre-mRNA is from a mutant CAPN3 gene, and the disease or condition is limb-girdle muscular dystrophy. In some embodiments the pre-mRNA is from a mutant ATXN3 gene, and the disease or condition is Spinocerebellar ataxia type 3. In some embodiments the pre-mRNA is from a mutant NOTCH3 gene, and the disease or condition is cerebral autosomal dominant arteriopathy with subcortical infarcts and leukoencephalopathy. In some embodiments the pre-mRNA is from a mutant COL4A5 gene, and the disease or condition is Alport syndrome. In some embodiments the pre-mRNA is from a mutant NF1 gene, and the disease or condition is Neurofibromatosis Type 1 (NF1). In some embodiments the pre-mRNA is from a mutant PCSK9 gene, and the disease or condition is familial hypercholesterolemia. In some embodiments or the pre-mRNA is from a mutant SCNN1A gene, and the disease or condition is pseudohypoaldosteronism type 1.

Generally, dosages, schedules, and routes of administration of the compositions (e.g., dRNA or construct comprising a nucleic acid encoding dRNA) may be determined according to the size and condition of the individual, and according to standard pharmaceutical practice. Exemplary routes of administration include intravenous, intra-arterial, intraperitoneal, intrapulmonary, intravesicular, intramuscular, intra-tracheal, subcutaneous, intraocular, intrathecal, or transdermal.

The exon skipping methods of the present application can not only be used in animal cells, for example mammalian cells, but also may be used in modification of RNAs of plant or fungi, for example, in plants or fungi that have endogenously expressed ADARs. The methods described herein can be used to generate genetically engineered plant and fungi with improved properties.

### V. Compositions, Kits and Articles of Manufacture

Also provided herein are compositions (such as pharmaceutical compositions) comprising any one of the dRNAs, constructs, libraries, or host cells having edited RNA as described herein.

In some embodiments, there is provided a pharmaceutical composition comprising any one of the dRNAs or constructs encoding the dRNA described herein, and a pharmaceutically acceptable carriers, excipients or stabilizers (Remington's Pharmaceutical Sciences 16^{th} edition, Osol, A. Ed. (1980)). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). In some embodiments, lyophilized formulations are provided. Pharmaceutical compositions to be used for in vivo administration must be sterile. This is readily accomplished by, e.g., filtration through sterile filtration membranes.

Further provided are kits useful for any one of the methods of RNA editing or methods of treatment described herein, comprising any one of the dRNAs, constructs, compositions, libraries, or edited host cells as described herein.

The kits of the present application are in suitable packaging. Suitable packaging includes, but is not limited to, vials, bottles, jars, flexible packaging (e.g., sealed Mylar or plastic bags), and the like. Kits may optionally provide additional components such as transfection or transduction reagents, cell culturing medium, buffers, and interpretative information.

The present application thus also provides articles of manufacture. The article of manufacture can comprise a container and a label or package insert on or associated with the container. Suitable containers include vials (such as sealed vials), bottles, jars, flexible packaging, and the like. In some embodiments, the container holds a pharmaceutical composition, and may have a sterile access port (for example the container may be an intravenous solution bag or a vial having a stopper pierceable by a hypodermic injection needle). The container holding the pharmaceutical composition may be a multi-use vial, which allows for repeat administrations (e.g. from 2-6 administrations) of the reconstituted formulation. Package insert refers to instructions customarily included in commercial packages of therapeutic products that contain information about the indications, usage, dosage, administration, contraindications and/or warnings concerning the use of such products. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kits or article of manufacture may include multiple unit doses of the pharmaceutical compositions and instructions for use, packaged in quantities sufficient for storage and use in pharmacies, for example, hospital pharmacies and compounding pharmacies.

### EXAMPLES

### Example 1 Modulation of Splicing of RELA and Trp53 Pre-mRNAs In Vitro

This example demonstrates that arRNA with recruited ADAR can be used to modulate splicing in vitro.

To assess RNA exon skipping on endogenous mRNA transcripts, HEK293T cells were seeded in six-well plates (8×10⁵ cells per well). Twenty-four hours later, cells were transfected with 4 μg of circular arRNA plasmids. The sequences of the control RNA, arRNA, and 5′ and 3′ ligation spacers for arRNA linker are listed in Table E1A. Forty-eight hours post transfection, cells were collected. Total RNA was isolated and reverse-transcribed into cDNA via RT-PCR. The targeted locus was PCR-amplified with the corresponding primers (Table E1A).

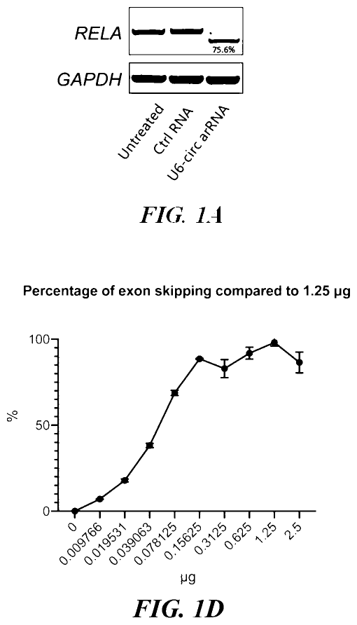

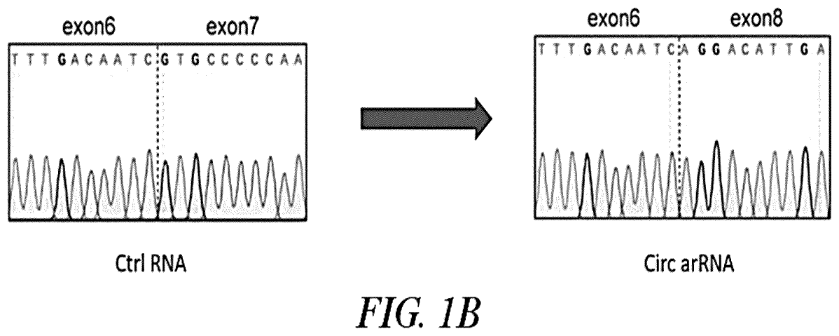

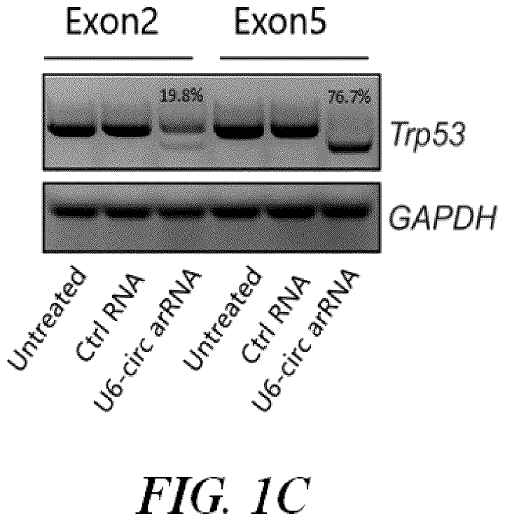

As shown in FIGS. 1A and 1B, exon 7 in the RELA pre-mRNA was skipped in the circ arRNA treated cells, resulting in shorter mature mRNA molecules whereas cells untreated or treated with the control RNA did not skip exon 7. The skipping efficiency was 75.6%.

TABLE ElA
Sequences of RELA- or Trp53-targeting arRNA and 5′ and 3′ ligation spacers (the
mismatch nucleotides are indicated by the lowercase letters)

| Target | Annotation | |
|---|---|---|
| Gene | (SEQ ID NO:) | Sequence |
| RELA | arRNA (SEQ ID | TGCCAGAGTTTCGGTTCACTCGGCAGATCTTGAGCTCG |
| | NO: 1) | GCAGTGTTGGGGGCACCcGAGGCAGTGAAAACAAGGG |
| | | AGGATGACCTGAGCCACGAGAGGTCCCCAGGGTGGC |
| | 5′ ligation spacer | AACCATGCCGACTGATGGCAG |
| | for arRNA linker | |
| | (SEQ ID NO: 2) | |
| | 3′ ligation spacer | CTGCCATCAGTCGGCGTGGACTGTAG |
| | for arRNA linker | |
| | (SEQ ID NO: 3) | |
| | Forward primer | CCCACGAGCTTGTAGGAAAGGA |
| | (SEQ ID NO: 10) | |
| | Reverse primer | CCTGGTCCCGTGAAATACACCT |
| | (SEQ ID NO: 11) | |
| Trp53 | arRNA targeting | GCAGGAACTGTTACACATGTAGTTGTAGTGGATGGTG |
| | Exon 2 (SEQ ID | GTACAGTCAGAGCCAACCcAGGAGATAACACAGGCCC |
| | NO: 4) | AAGATGAGGCCAGTGCGCCTTGGGGAGACCTGTGGCA |
| | arRNA targeting | TTCCAAGGCCTCATTCAGCTCTCGGAACATCTCGAAGC |
| | Exon 5 (SEQ ID | GCTCACGCCCACGGATCcGCAGCAACAGAGGAGGGGG |
| | NO: 12) | AGAAGTAAGTATATACACAGTACCTGAGTTAAAAGA |
| | 5′ ligation spacer | AACCATGCCGACTGATGGCAG |
| | for arRNA linker | |
| | (SEQ ID NO: 2) | |
| | 3′ ligation spacer | CTGCCATCAGTCGGCGTGGACTGTAG |
| | for arRNA linker | |
| | (SEQ ID NO: 3) | |
| | Forward primer | TGGAAGGAAATTTGCGTGTGG |
| | (SEQ ID NO: 13) | |
| | Reverse primer | TATGGCGGGAGGTAGACTGA |
| | (SEQ ID NO: 14) | |
| | Control RNA (SEQ | actacagttgctccgatatttaggctacgtcaataggcactaacttattggcgctggtgaac |
| | ID NO: 5) | ggacttcctctcgagtaccagaagatgactacaaaactcctttccattgcgagtatcggagt |
| | | ctggctcagtttggccagggaggcact |

As shown in FIG. 1C, both Exon 2 and Exon 5 in the Trp53 pre-mRNA were targeted by the circular arRNA, and exon skipping was observed for both with 19.8% and 76.7% efficiency, respectively.

Additionally, exon skipping efficiency was measured at different dosages of the RELA-targeting circular arRNA listed in Table E1A. 2×10⁵ HEK293T cells were transfected. As FIG. 1D shows, exon skipping efficiency shows a positive correlation with dosage.

### Example 2 the Effect of ADAR1 in Modulation of Splicing of Pre-mRNA In Vitro

This example shows that the presence of ADAR1 enhances the exon skipping efficiency of arRNA.

To assess RNA exon skipping on endogenous mRNA transcripts, HEK293T or HEK293T ADAR1 knockout (ADAR1^{-/-}) cells were seeded in six-well plates (8×10⁵ cells per well). Twenty-four hours later, cells were transfected with 4 μg of circular arRNA plasmids. For PPIB, the sequences of arRNA, and 5′ and 3′ ligation spacers for arRNA linker are listed in Table E2A, and the sequences used for RELA and the control RNA were the same as Example 1. Forty-eight hours post transfection, cells were collected. Total RNA was isolated and reverse-transcribed into cDNA via RT-PCR. The targeted locus was PCR-amplified with the corresponding primers (Table E2A).

TABLE E2A
Sequences of PPIB-targeting arRNA and 5′ and 3′ ligation spacers (the
mismatch nucleotides are indicated by the lowercase letters)

| Target | Annotation | |
|---|---|---|
| Gene | (SEQ ID NO:) | Sequence |
| PPIB | arRNA (SEQ ID | AGTGCTTCAGTTTGAAGTTCTCATCGGGGAAGCGCTCA |
| | NO: 6) | CCGTAGATGCTCTTTCCcGGGAAAAAAGACAGAGCAGG |
| | | TCAGGGGCGCTGGATTGCGCCAAACCAAGCAGACA |
| | 5′ ligation spacer | AACCATGCCGACTGATGGCAG |
| | for arRNA linker | |
| | (SEQ ID NO: 2) | |
| | 3′ ligation spacer | CTGCCATCAGTCGGCGTGGACTGTAG |
| | for arRNA linker | |
| | (SEQ ID NO: 3) | |
| | Forward primer | GAGCCCGCGAGCAACC |
| | (SEQ ID NO: 15) | |
| | Reverse primer | GGGCCTGTGGAATGTGAGGG |
| | (SEQ ID NO: 16) | |

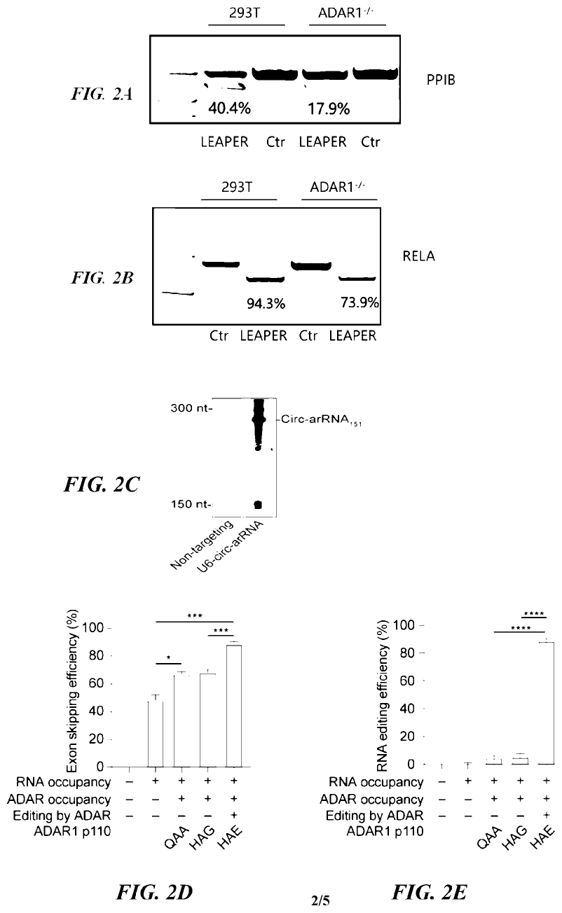

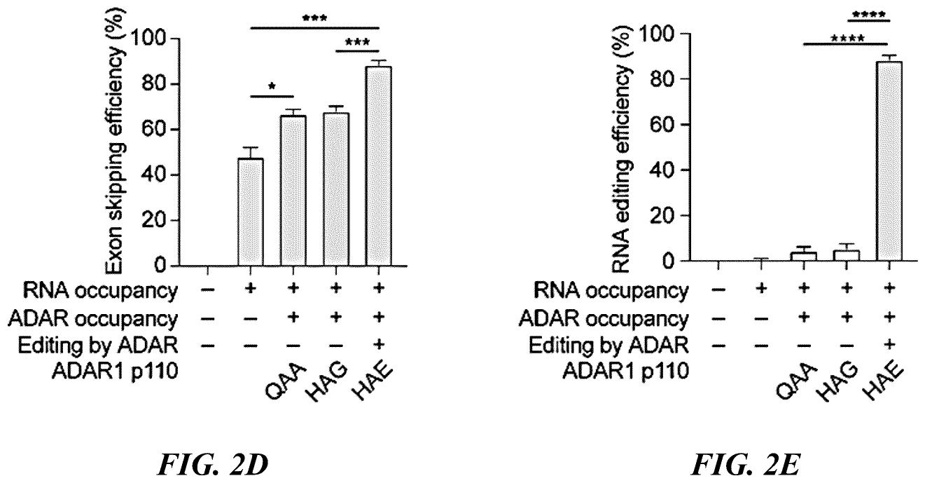

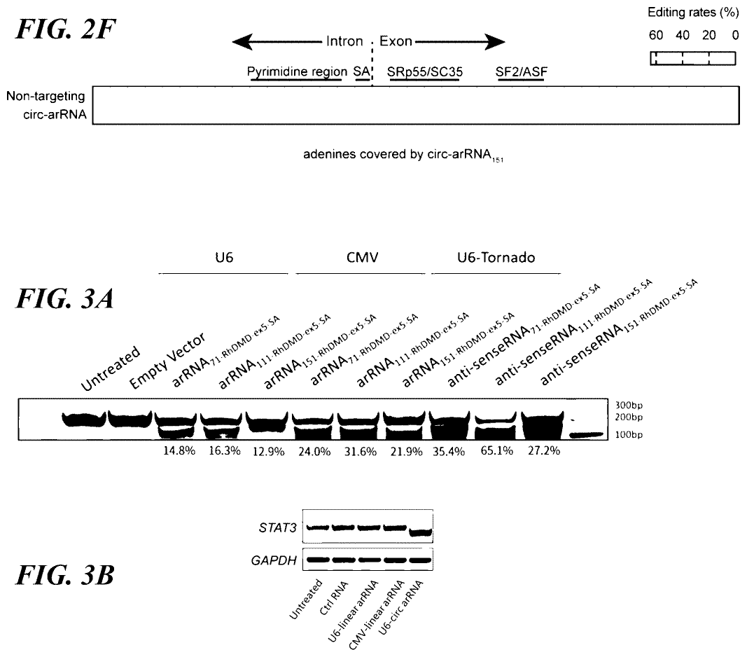

As shown in FIGS. 2A-2B, for both PPIB and RELA genes, the exon skipping efficiency was slower in ADAR1″-cells, suggesting the presence of ADAR1 protein in HEK293T cells enhances exon skipping using arRNA.

Considering the potential steric hindrance posed by circ-arRNA, the presence of arRNA in HEK293T cells was confirmed via Urea-PAGE (FIG. 2C), with expression levels comparable to those of 5S RNA. The arRNA sequence is ucgccguccagcucgaccaggaugggcaccaccccggugaacagcuccucgcccuugcucacuggcagagcccuccagcaucg cgagcaggcgcugccuccuccgccgcugccuccuccgccgcugccuccuccgcccugcaguuguaca (SEQ ID NO: 31).

Considering the potential steric hindrance posed by circ-arRNA, we evaluated the exon skipping efficiency in HEK293T ADAR1 knockout cells. Results revealed that exon skipping occurred even in cells without editing, albeit at a reduced efficiency compared to cells expressing ADAR1 (FIG. 2D). It was confirmed that no RNA editing activity was detected in this ADAR1 knockout line (FIG. 2E). Efficient exon skipping results from both ADAR-dependent and independent mechanisms. FIGS. 2D-2E show (1) RNA occupancy, (2) RNA and ADAR protein occupancy, (3) occupancy and editing altogether in mediating exon skipping. All experiments were done in HEK293T ADAR1−/− cells. Different ADAR forms (listed in Table E2B) were overexpressed by plasmids in this experiment. Efficiency difference between the ADAR active form (HAE) and the ADAR inactive forms (QAA and HAG) indicates the effect of RNA editing in exon skipping (FIGS. 2D-2E). The effect of occupation of both RNA and ADAR protein in exon skipping is illustrated in QAA and HAG (ADAR inactive forms). The effect of only RNA occupancy is illustrated by the second bar in FIG. 2D and FIG. 2E (without any form of ADAR1 expression).

We hypothesize that exon skipping results from a combination of factors, with ADAR playing a dual role. Firstly, ADAR's editing may alter sequences recognized by the spliceosome, including polypyrimidine tracts (PPTs), SA site, and exon splicing enhancers (ESE). Secondly, ADAR binding necessitates double-stranded RNA (dsRNA) formation, thereby physically obstructing the spliceosome due to steric hindrance. In essence, the combined effects of splicing element editing, ADAR occupancy, and circ-arRNA interaction facilitate exon skipping.

TABLE E2B
Sequences of different forms of ADAR

| Annotation | |
|---|---|
| (SEQ ID NO:) | Sequence |
| ADAR1 | ATGGCCGAGATCAAGGAGAAAATCTGCGACTATCTCTTCAATGTGTCTGACT |
| p110 QAA | CCTCTGCCCTGAATTTGGCTAAAAATATTGGCCTTACCAAGGCCCGAGATAT |
| (SEQ ID | AAATGCTGTGCTAATTGACATGGAAAGGCAGGGGGATGTCTATAGACAAGG |
| NO: 32) | GACAACCCCTCCCATATGGCATTTGACAGACAAGAAGCGAGAGAGGATGCA |
| | AATCAAGAGAAATACGAACAGTGTTCCTGAAACCGCTCCAGCTGCAATCCC |
| | TGAGACCAAAAGAAACGCAGAGTTCCTCACCTGTAATATACCCACATCAAA |
| | TGCCTCAAATAACATGGTAACCACAGAAAAAGTGGAGAATGGGCAGGAAC |
| | CTGTCATAAAGTTAGAAAACAGGCAAGAGGCCAGACCAGAACCAGCAAGA |
| | CTGAAACCACCTGTTCATTACAATGGCCCCTCAAAAGCAGGGTATGTTGACT |
| | TTGAAAATGGCCAGTGGGCCACAGATGACATCCCAGATGACTTGAATAGTA |
| | TCCGCGCAGCACCAGGTGAGTTTCGAGCCATCATGGAGATGCCCTCCTTCTA |
| | CAGTCATGGCTTGCCACGGTGTTCACCCTACAAGAAACTGACAGAGTGCCA |
| | GCTGAAGAACCCCATCAGCGGGCTGTTAGAATATGCCCAGTTCGCTAGTCA |
| | AACCTGTGAGTTCAACATGATAGAGCAGAGTGGACCACCCCATGAACCTCG |
| | ATTTAAATTCCAGGTTGTCATCAATGGCCGAGAGTTTCCCCCAGCTGAAGCT |
| | GGAAGCAAGAAAGTGGCCAAGCAGGATGCAGCTATGAAAGCCATGACAAT |
| | TCTGCTAGAGGAAGCCAAAGCCAAGGACAGTGGAAAATCAGAAGAATCAT |
| | CCCACTATTCCACAGAGAAAGAATCAGAGAAGACTGCAGAGTCCCAGACCC |
| | CCACCCCTTCAGCCACATCCTTCTTTTCTGGGAAGAGCCCCGTCACCACACT |
| | GCTTGAGTGTATGCACAAATTGGGGAACTCCTGCGAATTCCGTCTCCTGTCC |
| | AAAGAAGGCCCTGCCCATGAACCCAAGTTCCAATACTGTGTTGCAGTGGGA |
| | GCCCAAACTTTCCCCAGTGTGAGTGCTCCCAGCAAGAAAGTGGCAAAGCAG |
| | ATGGCCGCAGAGGAAGCCATGAAGGCCCTGCATGGGGAGGCGACCAACTC |
| | CATGGCTTCTGATAACCAGCCTGAAGGTATGATCTCAGAGTCACTTGATAAC |
| | TTGGAATCCATGATGCCCAACAAGGTCAGGAAGATTGGCGAGCTCGTGAGA |
| | TACCTGAACACCAACCCTGTGGGTGGCCTTTTGGAGTACGCCCGCTCCCATG |
| | GCTTTGCTGCTGAATTCAAGTTGGTCGACCAGTCCGGACCTCCTCACGAGCC |
| | CAAGTTCGTTTACCAAGCAAAAGTTGGGGGTCGCTGGTTCCCAGCCGTCTGC |
| | GCACACAGCAAGAAGCAAGGCAAGCAGGAAGCAGCAGATGCGGCTCTCCG |
| | TGTCTTGATTGGGGAGAACGAGAAGGCAGAACGCATGGGTTTCACAGAGGT |
| | AACCCCAGTGACAGGGGCCAGTCTCAGAAGAACTATGCTCCTCCTCTCAAG |
| | GTCCCCAGAAGCACAGCCAAAGACACTCCCTCTCACTGGCAGCACCTTCCA |
| | TGACCAGATAGCCATGCTGAGCCACCGGTGCTTCAACACTCTGACTAACAG |
| | CTTCCAGCCCTCCTTGCTCGGCCGCAAGATTCTGGCCGCCATCATTATGAAA |
| | AAAGACTCTGAGGACATGGGTGTCGTCGTCAGCTTGGGAACAGGGAATCGC |
| | TGTGTAAAAGGAGATTCTCTCAGCCTAAAAGGAGAAACTGTCAATGACTGC |
| | CAAGCTGCAATCATCTCCCGGAGAGGCTTCATCAGGTTTCTCTACAGTGAGT |
| | TAATGAAATACAACTCCCAGACTGCGAAGGATAGTATATTTGAACCTGCTA |
| | AGGGAGGAGAAAAGCTCCAAATAAAAAAGACTGTGTCATTCCATCTGTATA |
| | TCAGCACTGCTCCGTGTGGAGATGGCGCCCTCTTTGACAAGTCCTGCAGCGA |
| | CCGTGCTATGGAAAGCACAGAATCCCGCCACTACCCTGTCTTCGAGAATCCC |
| | AAACAAGGAAAGCTCCGCACCAAGGTGGAGAACGGAGAAGGCACAATCCC |
| | TGTGGAATCCAGTGACATTGTGCCTACGTGGGATGGCATTCGGCTCGGGGA |
| | GAGACTCCGTACCATGTCCTGTAGTGACAAAATCCTACGCTGGAACGTGCT |
| | GGGCCTGCAAGGGGCACTGTTGACCCACTTCCTGCAGCCCATTTATCTCAAA |
| | TCTGTCACATTGGGTTACCTTTTCAGCCAAGGGCATCTGACCCGTGCTATTT |
| | GCTGTCGTGTGACAAGAGATGGGAGTGCATTTGAGGATGGACTACGACATC |
| | CCTTTATTGTCAACCACCCCAAGGTTGGCAGAGTCAGCATATATGATTCCAA |
| | AAGGCAATCCGGGAAGACTAAGGAGACAAGCGTCAACTGGTGTCTGGCTGA |
| | TGGCTATGACCTGGAGATCCTGGACGGTACCAGAGGCACTGTGGATGGGCC |
| | ACGGAATGAATTGTCCCGGGTCTCCAAAAAGAACATTTTTCTTCTATTTAAG |
| | AAGCTCTGCTCCTTCCGTTACCGCAGGGATCTACTGAGACTCTCCTATGGTG |
| | AGGCCAAGAAAGCTGCCCGTGACTACGAGACGGCCAAGAACTACTTCAAAA |
| | AAGGCCTGAAGGATATGGGCTATGGGAACTGGATTAGCAAACCCCAGGAG |
| | GAAAAGAACTTTTATCTCTGCCCAGTA |
| ADAR1 | ATGGCCGAGATCAAGGAGAAAATCTGCGACTATCTCTTCAATGTGTCTGACT |
| p110 HAG | CCTCTGCCCTGAATTTGGCTAAAAATATTGGCCTTACCAAGGCCCGAGATAT |
| (SEQ ID | AAATGCTGTGCTAATTGACATGGAAAGGCAGGGGGATGTCTATAGACAAGG |
| NO: 33) | GACAACCCCTCCCATATGGCATTTGACAGACAAGAAGCGAGAGAGGATGCA |
| | AATCAAGAGAAATACGAACAGTGTTCCTGAAACCGCTCCAGCTGCAATCCC |
| | TGAGACCAAAAGAAACGCAGAGTTCCTCACCTGTAATATACCCACATCAAA |
| | TGCCTCAAATAACATGGTAACCACAGAAAAAGTGGAGAATGGGCAGGAAC |
| | CTGTCATAAAGTTAGAAAACAGGCAAGAGGCCAGACCAGAACCAGCAAGA |
| | CTGAAACCACCTGTTCATTACAATGGCCCCTCAAAAGCAGGGTATGTTGACT |
| | TTGAAAATGGCCAGTGGGCCACAGATGACATCCCAGATGACTTGAATAGTA |
| | TCCGCGCAGCACCAGGTGAGTTTCGAGCCATCATGGAGATGCCCTCCTTCTA |
| | CAGTCATGGCTTGCCACGGTGTTCACCCTACAAGAAACTGACAGAGTGCCA |
| | GCTGAAGAACCCCATCAGCGGGCTGTTAGAATATGCCCAGTTCGCTAGTCA |
| | AACCTGTGAGTTCAACATGATAGAGCAGAGTGGACCACCCCATGAACCTCG |
| | ATTTAAATTCCAGGTTGTCATCAATGGCCGAGAGTTTCCCCCAGCTGAAGCT |
| | GGAAGCAAGAAAGTGGCCAAGCAGGATGCAGCTATGAAAGCCATGACAAT |
| | TCTGCTAGAGGAAGCCAAAGCCAAGGACAGTGGAAAATCAGAAGAATCAT |
| | CCCACTATTCCACAGAGAAAGAATCAGAGAAGACTGCAGAGTCCCAGACCC |
| | CCACCCCTTCAGCCACATCCTTCTTTTCTGGGAAGAGCCCCGTCACCACACT |
| | GCTTGAGTGTATGCACAAATTGGGGAACTCCTGCGAATTCCGTCTCCTGTCC |
| | AAAGAAGGCCCTGCCCATGAACCCAAGTTCCAATACTGTGTTGCAGTGGGA |
| | GCCCAAACTTTCCCCAGTGTGAGTGCTCCCAGCAAGAAAGTGGCAAAGCAG |
| | ATGGCCGCAGAGGAAGCCATGAAGGCCCTGCATGGGGAGGCGACCAACTC |
| | CATGGCTTCTGATAACCAGCCTGAAGGTATGATCTCAGAGTCACTTGATAAC |
| | TTGGAATCCATGATGCCCAACAAGGTCAGGAAGATTGGCGAGCTCGTGAGA |
| | TACCTGAACACCAACCCTGTGGGTGGCCTTTTGGAGTACGCCCGCTCCCATG |
| | GCTTTGCTGCTGAATTCAAGTTGGTCGACCAGTCCGGACCTCCTCACGAGCC |
| | CAAGTTCGTTTACCAAGCAAAAGTTGGGGGTCGCTGGTTCCCAGCCGTCTGC |
| | GCACACAGCAAGAAGCAAGGCAAGCAGGAAGCAGCAGATGCGGCTCTCCG |
| | TGTCTTGATTGGGGAGAACGAGAAGGCAGAACGCATGGGTTTCACAGAGGT |
| | AACCCCAGTGACAGGGGCCAGTCTCAGAAGAACTATGCTCCTCCTCTCAAG |
| | GTCCCCAGAAGCACAGCCAAAGACACTCCCTCTCACTGGCAGCACCTTCCA |
| | TGACCAGATAGCCATGCTGAGCCACCGGTGCTTCAACACTCTGACTAACAG |
| | CTTCCAGCCCTCCTTGCTCGGCCGCAAGATTCTGGCCGCCATCATTATGAAA |
| | AAAGACTCTGAGGACATGGGTGTCGTCGTCAGCTTGGGAACAGGGAATCGC |
| | TGTGTAAAAGGAGATTCTCTCAGCCTAAAAGGAGAAACTGTCAATGACTGC |
| | CATGCCGGCATCATCTCCCGGAGAGGCTTCATCAGGTTTCTCTACAGTGAGT |
| | TAATGAAATACAACTCCCAGACTGCGAAGGATAGTATATTTGAACCTGCTA |
| | AGGGAGGAGAAAAGCTCCAAATAAAAAAGACTGTGTCATTCCATCTGTATA |
| | TCAGCACTGCTCCGTGTGGAGATGGCGCCCTCTTTGACAAGTCCTGCAGCGA |
| | CCGTGCTATGGAAAGCACAGAATCCCGCCACTACCCTGTCTTCGAGAATCCC |
| | AAACAAGGAAAGCTCCGCACCAAGGTGGAGAACGGAGAAGGCACAATCCC |
| | TGTGGAATCCAGTGACATTGTGCCTACGTGGGATGGCATTCGGCTCGGGGA |
| | GAGACTCCGTACCATGTCCTGTAGTGACAAAATCCTACGCTGGAACGTGCT |
| | GGGCCTGCAAGGGGCACTGTTGACCCACTTCCTGCAGCCCATTTATCTCAAA |
| | TCTGTCACATTGGGTTACCTTTTCAGCCAAGGGCATCTGACCCGTGCTATTT |
| | GCTGTCGTGTGACAAGAGATGGGAGTGCATTTGAGGATGGACTACGACATC |
| | CCTTTATTGTCAACCACCCCAAGGTTGGCAGAGTCAGCATATATGATTCCAA |
| | AAGGCAATCCGGGAAGACTAAGGAGACAAGCGTCAACTGGTGTCTGGCTGA |
| | TGGCTATGACCTGGAGATCCTGGACGGTACCAGAGGCACTGTGGATGGGCC |
| | ACGGAATGAATTGTCCCGGGTCTCCAAAAAGAACATTTTTCTTCTATTTAAG |
| | AAGCTCTGCTCCTTCCGTTACCGCAGGGATCTACTGAGACTCTCCTATGGTG |
| | AGGCCAAGAAAGCTGCCCGTGACTACGAGACGGCCAAGAACTACTTCAAAA |
| | AAGGCCTGAAGGATATGGGCTATGGGAACTGGATTAGCAAACCCCAGGAG |
| | GAAAAGAACTTTTATCTCTGCCCAGTA |
| ADAR1 | ATGGCCGAGATCAAGGAGAAAATCTGCGACTATCTCTTCAATGTGTCTGACT |
| p110 HAE | CCTCTGCCCTGAATTTGGCTAAAAATATTGGCCTTACCAAGGCCCGAGATAT |
| (active | AAATGCTGTGCTAATTGACATGGAAAGGCAGGGGGATGTCTATAGACAAGG |
| form) (SEQ | GACAACCCCTCCCATATGGCATTTGACAGACAAGAAGCGAGAGAGGATGCA |
| ID NO: 34) | AATCAAGAGAAATACGAACAGTGTTCCTGAAACCGCTCCAGCTGCAATCCC |
| | TGAGACCAAAAGAAACGCAGAGTTCCTCACCTGTAATATACCCACATCAAA |
| | TGCCTCAAATAACATGGTAACCACAGAAAAAGTGGAGAATGGGCAGGAAC |
| | CTGTCATAAAGTTAGAAAACAGGCAAGAGGCCAGACCAGAACCAGCAAGA |
| | CTGAAACCACCTGTTCATTACAATGGCCCCTCAAAAGCAGGGTATGTTGACT |
| | TTGAAAATGGCCAGTGGGCCACAGATGACATCCCAGATGACTTGAATAGTA |
| | TCCGCGCAGCACCAGGTGAGTTTCGAGCCATCATGGAGATGCCCTCCTTCTA |
| | CAGTCATGGCTTGCCACGGTGTTCACCCTACAAGAAACTGACAGAGTGCCA |
| | GCTGAAGAACCCCATCAGCGGGCTGTTAGAATATGCCCAGTTCGCTAGTCA |
| | AACCTGTGAGTTCAACATGATAGAGCAGAGTGGACCACCCCATGAACCTCG |
| | ATTTAAATTCCAGGTTGTCATCAATGGCCGAGAGTTTCCCCCAGCTGAAGCT |
| | GGAAGCAAGAAAGTGGCCAAGCAGGATGCAGCTATGAAAGCCATGACAAT |
| | TCTGCTAGAGGAAGCCAAAGCCAAGGACAGTGGAAAATCAGAAGAATCAT |
| | CCCACTATTCCACAGAGAAAGAATCAGAGAAGACTGCAGAGTCCCAGACCC |
| | CCACCCCTTCAGCCACATCCTTCTTTTCTGGGAAGAGCCCCGTCACCACACT |
| | GCTTGAGTGTATGCACAAATTGGGGAACTCCTGCGAATTCCGTCTCCTGTCC |
| | AAAGAAGGCCCTGCCCATGAACCCAAGTTCCAATACTGTGTTGCAGTGGGA |
| | GCCCAAACTTTCCCCAGTGTGAGTGCTCCCAGCAAGAAAGTGGCAAAGCAG |
| | ATGGCCGCAGAGGAAGCCATGAAGGCCCTGCATGGGGAGGCGACCAACTC |
| | CATGGCTTCTGATAACCAGCCTGAAGGTATGATCTCAGAGTCACTTGATAAC |
| | TTGGAATCCATGATGCCCAACAAGGTCAGGAAGATTGGCGAGCTCGTGAGA |
| | TACCTGAACACCAACCCTGTGGGTGGCCTTTTGGAGTACGCCCGCTCCCATG |
| | GCTTTGCTGCTGAATTCAAGTTGGTCGACCAGTCCGGACCTCCTCACGAGCC |
| | CAAGTTCGTTTACCAAGCAAAAGTTGGGGGTCGCTGGTTCCCAGCCGTCTGC |
| | GCACACAGCAAGAAGCAAGGCAAGCAGGAAGCAGCAGATGCGGCTCTCCG |
| | TGTCTTGATTGGGGAGAACGAGAAGGCAGAACGCATGGGTTTCACAGAGGT |
| | AACCCCAGTGACAGGGGCCAGTCTCAGAAGAACTATGCTCCTCCTCTCAAG |
| | GTCCCCAGAAGCACAGCCAAAGACACTCCCTCTCACTGGCAGCACCTTCCA |
| | TGACCAGATAGCCATGCTGAGCCACCGGTGCTTCAACACTCTGACTAACAG |
| | CTTCCAGCCCTCCTTGCTCGGCCGCAAGATTCTGGCCGCCATCATTATGAAA |
| | AAAGACTCTGAGGACATGGGTGTCGTCGTCAGCTTGGGAACAGGGAATCGC |
| | TGTGTAAAAGGAGATTCTCTCAGCCTAAAAGGAGAAACTGTCAATGACTGC |
| | CATGCAGAAATAATCTCCCGGAGAGGCTTCATCAGGTTTCTCTACAGTGAGT |
| | TAATGAAATACAACTCCCAGACTGCGAAGGATAGTATATTTGAACCTGCTA |
| | AGGGAGGAGAAAAGCTCCAAATAAAAAAGACTGTGTCATTCCATCTGTATA |
| | TCAGCACTGCTCCGTGTGGAGATGGCGCCCTCTTTGACAAGTCCTGCAGCGA |
| | CCGTGCTATGGAAAGCACAGAATCCCGCCACTACCCTGTCTTCGAGAATCCC |
| | AAACAAGGAAAGCTCCGCACCAAGGTGGAGAACGGAGAAGGCACAATCCC |
| | TGTGGAATCCAGTGACATTGTGCCTACGTGGGATGGCATTCGGCTCGGGGA |
| | GAGACTCCGTACCATGTCCTGTAGTGACAAAATCCTACGCTGGAACGTGCT |
| | GGGCCTGCAAGGGGCACTGTTGACCCACTTCCTGCAGCCCATTTATCTCAAA |
| | TCTGTCACATTGGGTTACCTTTTCAGCCAAGGGCATCTGACCCGTGCTATTT |
| | GCTGTCGTGTGACAAGAGATGGGAGTGCATTTGAGGATGGACTACGACATC |
| | CCTTTATTGTCAACCACCCCAAGGTTGGCAGAGTCAGCATATATGATTCCAA |
| | AAGGCAATCCGGGAAGACTAAGGAGACAAGCGTCAACTGGTGTCTGGCTGA |
| | TGGCTATGACCTGGAGATCCTGGACGGTACCAGAGGCACTGTGGATGGGCC |
| | ACGGAATGAATTGTCCCGGGTCTCCAAAAAGAACATTTTTCTTCTATTTAAG |
| | AAGCTCTGCTCCTTCCGTTACCGCAGGGATCTACTGAGACTCTCCTATGGTG |
| | AGGCCAAGAAAGCTGCCCGTGACTACGAGACGGCCAAGAACTACTTCAAAA |
| | AAGGCCTGAAGGATATGGGCTATGGGAACTGGATTAGCAAACCCCAGGAG |
| | GAAAAGAACTTTTATCTCTGCCCAGTA |

To assess RNA editing in the sequence flanking splicing acceptor (SA) in intron 4 of DMD, a reporter containing intron 4-exon 5-intron 5 was construct into pcDNA3.1 backbone. A truncated version of intron was adopted to reduce its long size. HEK293T cells were transfected by circ-arRNA (SEQ ID NO: 9) and this reporter to detect editing rates.

In targeting a single base for precise editing, removing uridines that pair with non-target adenosines helped reduce bystander editing. However, in our scenario, the bystander editing that occurs within the dsRNA, formed between the arRNA and its target, actually benefits exon skipping by further impeding spliceosome recognition (FIG. 2F).

### Example 3 the Effect of Different Promoters and arRNA Lengths and Structures in Modulation of Pre-mRNA Splicing In Vitro

In this example, different promoters, arRNA structures, and targeting RNA lengths were tested for exon skipping efficiencies for DMD and STAT3.

Different arRNAs were designed to target Exon 5 of the DMD pre-mRNA in Cos7 cells comprising either a U6 or a CMV promoter, a length of 71 nt, 111 nt, or 151 nt, with or without Tornado expression system. The Tornado expression system was described in detail in WO2021008447A1, incorporated herein in its entirety by reference. Tornado-expressed transcripts contain an RNA of interest flanked by Twister ribozymes. The ribozymes rapidly undergo autocatalytic cleavage, leaving termini that are ligated by the ubiquitous endogenous RNA ligase RtcB. The sequences of arRNAs, and 5′ and 3′ ligation spacers for arRNA linker are listed in Table E3A. Cos7 cells were cultured and transfected as described in Example 1 and Example 2, and RNA exon skipping on DMD mRNA transcripts was assessed similarly.

TABLE E3A
Sequences of DMD-targeting arRNAs and 5′ and 3′ ligation spacers (the
mismatch nucleotides are indicated by the lowercase letters)

| Target | Annotation | |
|---|---|---|
| Gene | (SEQ ID NO:) | Sequence |
| DMD | arRNA 71 nt | CGATGTCAGTACTTCCAATATTCACTAAATCAACCCGTT |
| | (SEQ ID NO: 7) | AAAGAGAAGGGGAAAACATTTGAAGGTAAGAA |
| | arRNA 111 nt | cagTTTATGATTTCCATCTACGATGTCAGTACTTCCAATA |
| | (SEQ ID NO: 8) | TTCACTAAATCAACCCGTTAAAGAGAAGGGGAAAACAT |
| | | TTGAAGGTAAGAAACCAAATGCCTAGTTGCAAT |
| | arRNA 151 nt | TTCCAAATCAAACCAAGAgtcagTTTATGATTTCCATCTAC |
| | (SEQ ID NO: 9) | GATGTCAGTACTTCCAATATTCACTAAATCAACCCGTTA |
| | | AAGAGAAGGGGAAAACATTTGAAGGTAAGAAACCAAA |
| | | TGCCTAGTTGCAATAACAATAATAAAAATGTGAA |
| | 5′ ligation spacer | AACCATGCCGACTGATGGCAG |
| | for arRNA linker | |
| | (SEQ ID NO: 2) | |
| | 3′ ligation spacer | CTGCCATCAGTCGGCGTGGACTGTAG |
| | for arRNA linker | |
| | (SEQ ID NO: 3) | |
| STAT3 | STAT3 arRNA | GAAUCUAAAGUGCGGGGGGACAUCGGCAGGUCAAUGG |
| | (SEQ ID NO: 46) | UAUUGCUGCAGGUCGUUCcGUAGGAAAUGGGGGGCAG |
| | | CAGGAGGGGAAACGGGGGGUUGACAAGACACAAUGGA |

As shown in FIG. 3A, across different promoters and expression systems, the arRNA with a length of 111 nt consistently showed the highest exon skipping efficiency, and the Tornado expression system with a U6 promoter performed better than groups without the Tornado expression system.

Different arRNAs were designed to target STAT3 pre-mRNA. As shown in FIG. 3B, both the linear and circular arRNA were able to induce exon skipping, while the circular arRNA with a U6 promoter showed the highest exon skipping efficiency.

### Example 4 Modulation of Pre-mRNA Splicing In Vivo

This example shows that dystrophin can be restored by modulating splicing of DMD pre-mRNA in vivo.

DMD rhesus monkey model comprises a 5 bp deletion in the coding region of DMD, causing the DMD protein to be non-functional. Circ-arRNAs were packaged in MyoAAV4E (Tabebordbar et al., Cell, 2022) by PackGene Biotech. The sequence of the arRNA is set forth in SEQ ID NO: 9. The titer of AAV is 1×10¹³ GC/ml. MyoAAV4E was injected into the DMD monkey by intravenous injection at 5×10¹³ GC/kg. The monkeys were monitored for the duration of the experiment.

Eight weeks after injection, the monkeys' triceps, and deltoid were harvested. Harvested monkey tissues were homogenized in 1 ml of TRIzol, and RNA was extracted by the chloroform extraction method and reverse-transcribed. PCR-amplified products were analyzed by agarose electrophoresis to measure exon skipping efficiency and NGS to measure editing rates. Expression levels of arRNA in the tissues were measured by qPCR.

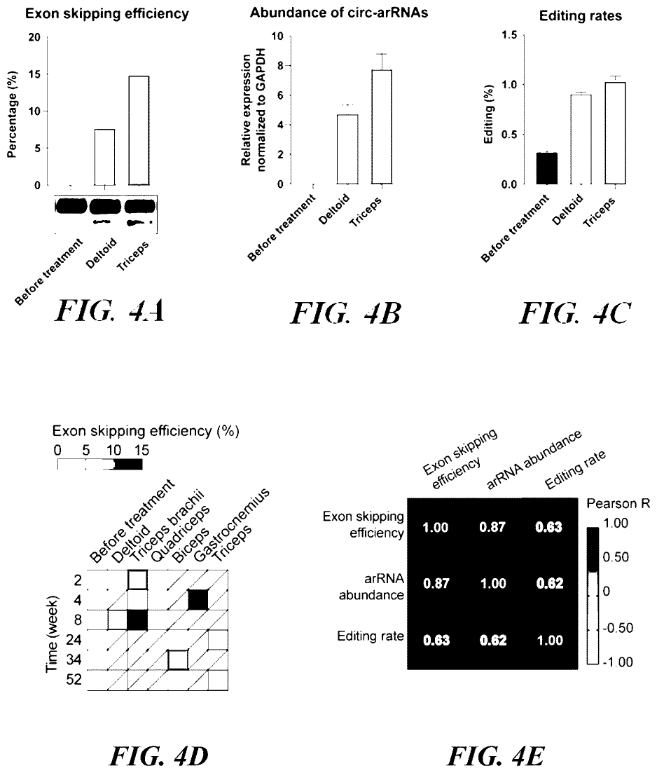

The results showed that eight weeks post injection, the exon skipping efficiency in the tissues varied between about 5% to about 15% (FIG. 4A), the circ-arRNAs were found in the tissues in abundance (FIG. 4B), and the editing rates in the tissues were found to be around 1.0% (FIG. 4C). In addition, the serum creatinine concentration decreased by 70%, suggesting that the function of the DMD protein was restored by 70%.

The different exon 5 skipping efficiencies in different muscles is attributed to variable expression levels of circ-arRNA (FIG. 4D), influenced by differing infection efficiencies. Collectively, these findings highlight strong positive correlations between the abundance of circ-arRNA, the efficiency of editing at the targeted SA site, and exon skipping success (FIG. 4E).

### Example 5 the Effect of Different Lengths and Target Sites in Modulation of Pre-mRNA Splicing

In this example, different targeting RNA lengths and targeting sites were tested for exon skipping efficiencies for DMD.

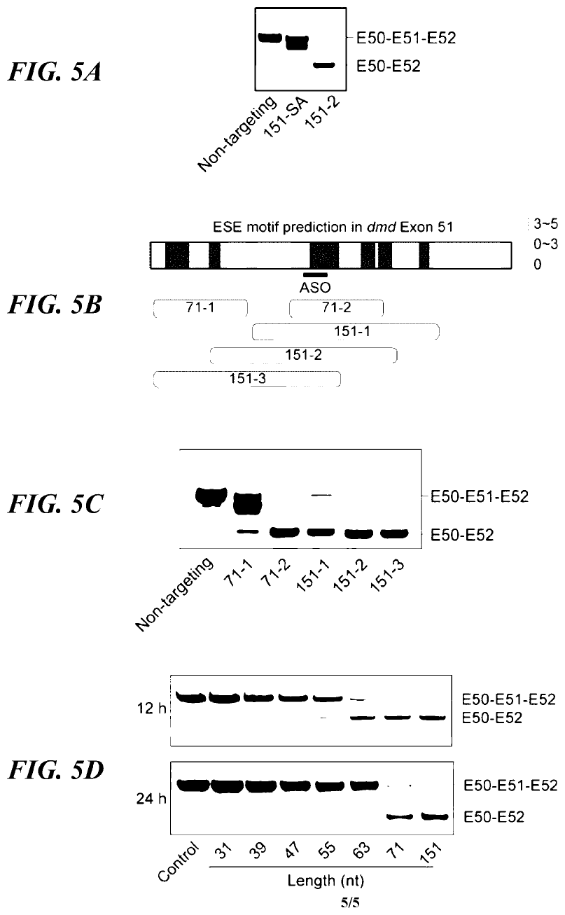

Different arRNAs were designed for modulating of splicing of exon 51 in the DMD pre-mRNA. Prediction of exon splicing enhancer motifs was performed by an online tool (http://krainer01.cshl.edu/cgi-bin/tools/ESE3/esefinder.cgi). The arRNA sequences are provided in Table ESA, and are illustrated in FIG. 5B.

First, the exon skipping efficiency of arRNAs targeting either the SA site of intron 50 or other target sites were assessed. It was observed that targeting splicing elements beyond the SA sites, such as exonic splicing enhancer sequences, could also induce exon skipping at native sites (FIGS. 5A-5C).

Additional arRNAs were designed to further assess the effect of arRNA length. The arRNA sequences are provided in Table E5B, and 71-2 (SEQ ID NO: 40) and 151-2 (SEQ ID NO: 37) were also assessed in this experiment, and was represented by “71” and “151” on FIG. 5D, respectively. It was observed that within the range of 31 nt to 151 nt, longer circ-arRNAs lead to higher exon skipping efficiencies (FIG. 5D).

TABLE ESA
Sequences of DMD-targeting arRNAs

| Target | Annotation | |
|---|---|---|
| Gene | (SEQ ID NO:) | Sequence |
| DMD | 151-SA (SEQ ID | CAUUUCUAGUUUGGAGAUGACAGUUUCCUUAGUAACCA |
| | NO: 35) | CAGAUUGUGUCACUAGAGUAACAGUCUGACUGGCAGCU |
| | | AGUGUUUUUGAAAGAAAGAAGAAAAGAAAAAAAAAAGG |
| | | GAAUAUGAGAAACACAAGCAAAAGAGCCAAUUUUAUA |
| | 151-1 (SEQ ID | ACCCACCAUCACUCUCUGUGAUUUUAUAACUCGAUCAAG |
| | NO: 36) | CAGAGACAGCCAGUCUGUAAGUUCUGUCCAAGCUCGGUU |
| | | GAAGUCUGCCAGUGCAGGUACCUCCAACAGCAAAGAAGA |
| | | UGGCAUUUCUAGUUUGGAGAUGACAGUUUCCUUA |
| | 151-2 (SEQ ID | GCAGAGACAGCCAGUCUGUAAGUUCUGUCCAAGCUCGGU |
| | NO: 37) | UGAAGUCUGCCAGUGCAGGUACCUCCAACAGCAAAGAAG |
| | | AUGGCAUUUCUAGUUUGGAGAUGACAGUUUCCUUAGUA |
| | | ACCACAGAUUGUGUCACUAGAGUAACAGUCUGACU |
| | 151-3 (SEQ ID | CAGUCUGUAAGUUCUGUCCAAGCUCGGUUGAAGUCUGCC |
| | NO: 38) | AGUGCAGGUACCUCCAACAGCAAAGAAGAUGGCAUUUCU |
| | | AGUUUGGAGAUGACAGUUUCCUUAGUAACCACAGAUUG |
| | | UGUCACUAGAGUAACAGUCUGACUGGCAGCUAGUG |
| | 71-1 (SEQ ID | UUCUAGUUUGGAGAUGACAGUUUCCUUAGUAACCACAG |
| | NO: 39) | AUUGUGUCACUAGAGUAACAGUCUGACUGGCAG |
| | 71-2 (SEQ ID | CAAGCAGAGACAGCCAGUCUGUAAGUUCUGUCCAAGCUC |
| | NO: 40) | GGUUGAAGUCUGCCAGUGCAGGUACCUCCAAC |

TABLE E5B
Sequences of DMD-targeting arRNAs

| Target | Annotation | |
|---|---|---|
| Gene | (SEQ ID NO:) | Sequence |
| DMD | 31 (SEQ ID NO: | UCCAAGCUCGGUUGAAGUCUGCCAGUGCAGG |
| | 41) | |
| | 39 (SEQ ID NO: | GUUCUGUCCAAGCUCGGUUGAAGUCUGCCAGUGCAGGU |
| | 42) | A |
| | 47 (SEQ ID NO: | GUCUGUAAGUUCUGUCCAAGCUCGGUUGAAGUCUGCCA |
| | 43) | GUGCAGGUA |
| | 55 (SEQ ID NO: | GACAGCCAGUCUGUAAGUUCUGUCCAAGCUCGGUUGAA |
| | 44) | GUCUGCCAGUGCAGGUA |
| | 63 (SEQ ID NO: | GACAGCCAGUCUGUAAGUUCUGUCCAAGCUCGGUUGAA |
| | 45) | GUCUGCCAGUGCAGGUACCUCCAAC |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### EXEMPLARY EMBODIMENTS

Embodiment 1. A method of modulating splicing of a target exon in a pre-mRNA in a host cell, the pre-mRNA comprising the target exon flanked by a 5′ intron and a 3′ intron, wherein the method comprises introducing a deaminase-recruiting RNA (dRNA) or a construct comprising a nucleic acid encoding the dRNA into the host cell, wherein: (1) dRNA comprises a targeting RNA sequence that is at least partially complementary to a target sequence in the pre-mRNA comprising a portion of the target exon, a portion of the 5′ intron, and/or a portion of the 3′ intron; and (2) the dRNA recruits an adenosine deaminase acting on RNA (ADAR) to the target exon, the 5′ intron, and/or the 3′ intron; wherein the recruitment of the ADAR modulate the splicing of the target exon in the pre-mRNA.

Embodiment 2. The method of Embodiment 1, wherein the recruitment of the ADAR blocks access of one or more splicing factors to a splicing site for the target exon, thereby modulating the splicing of the target exon in the pre-mRNA.

Embodiment 3. The method of Embodiment 2, wherein the recruitment of the ADAR does not deaminate a target adenosine in the 5′ intron or the 3′ intron.

Embodiment 4. The method of Embodiment 1 or 2, wherein the recruitment of the ADAR deaminates a target adenosine in the 5′ intron or the 3′ intron, thereby modulating the splicing of the target exon in the pre-mRNA.

Embodiment 5. The method of any one of Embodiments 1-4, wherein the pre-mRNA is spliced in the host cell to form a mature mRNA not including the target exon.

Embodiment 6. The method of any one of Embodiments 1-4, wherein the pre-mRNA is spliced in the host cell to form a mature mRNA including the target exon.

Embodiment 7. The method of any one of Embodiments 1-6, wherein the target sequence comprises a 3′ splice site of the 5′ intron comprising an AG splicing sequence.

Embodiment 8. The method of Embodiment 6, wherein the targeting sequence comprises a mismatch nucleotide opposite to the adenosine in the AG splicing sequence, thereby inducing deamination at the adenosine upon recruitment of the ADAR.

Embodiment 9. The method of Embodiment 6, wherein the targeting sequence does not comprise a mismatch nucleotide opposite to the adenosine in the AG splicing sequence.

Embodiment 10. The method of any one of Embodiments 1-9, wherein the target sequence comprises a branch point of the 5′ intron.

Embodiment 11. The method of Embodiment 10, wherein the targeting RNA sequence comprises a mismatch nucleotide opposite to the adenosine in the branch point, thereby inducing deamination at the adenosine upon recruitment of the ADAR.

Embodiment 12. The method of Embodiment 10, wherein the targeting RNA sequence does not comprise a mismatch nucleotide opposite to the adenosine in the branch point.

Embodiment 13. The method of any one of Embodiments 1-12, wherein the target sequence comprises a pyrimidine-rich sequence of the 5′ intron.

Embodiment 14. The method of any one of Embodiments 1-13, wherein the target sequence comprises an exonic splicing enhancer sequence of the target exon.

Embodiment 15. The method of any one of Embodiments 1-14, wherein the targeting RNA sequence is more than about 30 nucleotides in length.

Embodiment 16. The method of Embodiment 15, wherein the targeting RNA sequence is about 70 to about 150 nucleotides in length.

Embodiment 17. The method of any one of Embodiments 1-16, wherein the dRNA further comprises a 3′ ligation sequence and a 5′ ligation sequence.

Embodiment 18. The method of any one of Embodiments 1-17, wherein the dRNA is a circRNA.

Embodiment 19. The method of any one of Embodiments 1-17, wherein the dRNA is a linear RNA.

Embodiment 20. The method of Embodiment 19, wherein the dRNA is a linear RNA that forms a circRNA within the host cell.

Embodiment 21. The method of Embodiment 19 or 20, wherein the method comprises introducing a construct comprising a nucleic acid encoding the dRNA into the host cell.

Embodiment 22. The method of Embodiment 21, wherein the construct further comprises a 3′ twister ribozyme sequence linked to the 3′ end of the nucleic acid encoding the dRNA and a 5′ twister ribozyme sequence linked to the 5′ end of the nucleic acid encoding the dRNA.

Embodiment 23. The method of any one of Embodiments 21-22, wherein the construct is a viral vector or a plasmid.

Embodiment 24. The method of Embodiment 23, wherein the construct is an AAV vector, lentiviral vector, adenoviral vector, RNA replicon, pox virus vector, enteric virus vector, Venezuelan Equine Encephalitis virus vector, Semliki Forest Virus vector, or Tobacco Mosaic Virus vector.

Embodiment 25. The method of any one of Embodiments 1-24, wherein the dRNA further comprises an ADAR recruiting domain.

Embodiment 26. The method of any one of Embodiments 1-24, wherein the dRNA does not comprise an ADAR recruiting domain.

Embodiment 27. The method of any one of Embodiments 1-26, wherein the method comprises introduction of at least two dRNAs or constructs comprising a nucleic acid encoding the dRNA into the host cell, each comprising a targeting RNA sequence that is at least partially complementary to a different target sequence in the pre-mRNA.

Embodiment 28. The method of any one of Embodiments 1-27, wherein the dRNA is not chemically modified.

Embodiment 29. The method of any one of Embodiments 1-20 and 25-27, wherein the dRNA is chemically modified.

Embodiment 30. The method of any one of Embodiments 1-29, wherein the pre-mRNA is from a gene selected from the group consisting of dystrophin, RELA, Trp53, PPIB, STAT3, SMN1, CFTR, beta-globin, Factor VIII, Factor IX, TP53, mesenchymal epithelial transition factor receptor (MET), KRAS, RAB7A, ABCA4, SERPINA1, HEXA, LRRK2, SNCA, DMD, APP, MAPT, CFTR, ALAS1, ATP7B, HFE, LIP A, CEP290, USH2A, CD274, HTT, COL7A1, MS4A, SGCG, ATXN3, NOTCH3, COL4A5, NF1, PCSK9 start site, SCNN1A, a collagen VI alpha 1 gene (COL6A1), a myotubular myopathy 1 gene (MTM1), a dysferlin gene (DYSF), a laminin-alpha 2 gene (LAMA2), an emery-dreyfuss muscular dystrophy gene (EMD), a calpain 3 gene (CAPN3), and superoxide dismutase 1 (SOD1).

Embodiment 31. The method of Embodiment 30, wherein: (1) the pre-mRNA is from a dystrophin gene, and wherein the target exon is selected from the group consisting of exons 3, 4, 5, 8, 10-16, 19-40, 42-44, 46, 47, and 50-53; (2) the pre-mRNA is from an SMN1 gene and wherein the target exon is exon 7; (3) the pre-mRNA is from a CFTR gene, and wherein the target exon is selected from the group consisting of exons 10, 11, 12, and 13; (4) the pre-mRNA is from a beta-globin gene and wherein the target exon is selected from the group consisting of exons 51, 44, 45, and 27; (5) the pre-mRNA is from a Factor VIII gene, and wherein the target exon is selected from the group consisting of exons 5, 6, 7, and 8; (6) the pre-mRNA is from a Factor IX gene, and wherein the target exon is exon 2; (7) the pre-mRNA is from a RELA gene, and wherein the target exon is exon 7; (8) the pre-mRNA is from a Trp53 genes, and wherein the target exon is selected from the group consisting of exons 2 and 5; and/or (9) the pre-mRNA is from a PPIB gene, and wherein the target exon is exon 3.

Embodiment 32. The method of any one of Embodiments 1-31, wherein the host cell is an eukaryotic cell.

Embodiment 33. A method of treating or preventing a disease of an individual, comprising inducing exon skipping in a pre-mRNA of a host cell in the individual according to the method of any one of Embodiments 1-32.

Embodiment 34. The method of Embodiment 33, wherein: (1) the pre-mRNA is from a mutant dystrophin gene, and the disease or condition is Duchenne muscular dystrophy (DMD); (2) the pre-mRNA is from a mutant SMN1 gene, and the disease or condition is Spinal muscular atrophy (SMA); (3) the pre-mRNA is from a mutant CFTR gene, and the disease or condition is Cystic fibrosis (CF); (4) the pre-mRNA is from a mutant beta-globin gene, and the disease or condition is Beta-thalassemia; or (5) the pre-mRNA is from a mutant Factor VIII or Factor IX gene, and the disease or condition is hemophilia.

Embodiment 35. The method of Embodiment 33 or 34, wherein the dRNA or construct encoding the dRNA is introduced into the individual intravenously.

Embodiment 36. A ribonucleic acid (RNA) molecule, comprising a targeting RNA sequence complementary to a target RNA sequence, wherein the target RNA sequence comprises at least one of a 5′ splice site (5′ ss), a polypyrimidine tract, a branchpoint and a 3′ splice site (3′ ss) of a pre-messenger RNA (pre-mRNA), and the RNA molecule is capable of recruiting an adenosine deaminase acting on RNA (ADAR) to the target RNA sequence.

Embodiment 37. The RNA molecule of Embodiment 36, wherein the RNA molecule is a circular RNA.

Embodiment 38. The RNA molecule of Embodiment 36 or 37, further comprising a 3′ ligation sequence and a 5′ ligation sequence.

Embodiment 39. The RNA molecule of Embodiment 36 or 38, wherein the RNA molecule is a linear RNA capable of forming a circular RNA, preferably wherein the RNA molecule is capable of being circularized by RNA ligase RtcB in a host cell.

Embodiment 40. The RNA molecule of any one of Embodiments 36-39, wherein the targeting RNA sequence has a length of at least 30 nucleotides, such as at least 40, 50, 60, 70, 80, 90, 100, 110, 120, 130, 140, 150, 160, 170, 180, 190, 200, or more nucleotides.

Embodiment 41. The RNA molecule of any one of Embodiments 36-40, wherein the target RNA sequence comprises at least one, two, or three of the 3′ splice site (3′ ss), the polypyrimidine tract, and the branchpoint.

Embodiment 42. The RNA molecule of any one of Embodiments 36-41, wherein a. the 3′ ss comprises the nucleotide sequence of YAG; b. the polypyrimidine tract has a length of 4-25 nucleotides, such as 4, 5, 6, 7, 8, 9, 10, 11, 12, 13, 14, 15, 16, 17, 18, 19, 20, 21, 22, 23, 24 or 25 nucleotides; and/or c. the branchpoint comprises the nucleotide sequence of YUNAY, such as CURAY, YNCURAY, YNCURAC or YNYURAY, wherein Y is a pyrimidine, N is any nucleotide, and R is a purine.

Embodiment 43. The RNA molecule of any one of Embodiments 36-42, wherein the target RNA sequence comprises, in 5′ to 3′ direction: a. the branchpoint comprising the nucleotide sequence of YNYURAY and the polypyrimidine tract having a length of 10-20 nucleotides; b. the polypyrimidine tract having a length of 10-20 nucleotides and the 3′ ss comprising the nucleotide sequence of YAG; or c. the branchpoint comprising the nucleotide sequence of YNYURAY, the polypyrimidine tract having a length of 10-20 nucleotides, and the 3′ ss comprising the nucleotide sequence of YAG, optionally wherein the target RNA sequence further comprises the 5′ splice site (5′ ss).

Embodiment 44. A system for modulating splicing of a target exon in a pre-mRNA in a host cell, the pre-mRNA comprising the target exon flanked by a 5′ intron and a 3′ intron, wherein the system comprises a dRNA or a construct comprising a nucleic acid encoding the dRNA, wherein: (1) dRNA comprises a targeting RNA sequence that is at least partially complementary to a target sequence in the pre-mRNA comprising a portion of the target exon, a portion of the 5′ intron, and/or a portion of the 3′ intron; and (2) the dRNA is capable of recruiting an adenosine deaminase acting on RNA (ADAR) to the target exon, the 5′ intron, and/or the 3′ intron.

Embodiment 45. The system of Embodiment 44, wherein the target sequence comprises a 3′ splice site of the 5′ intron comprising an AG splicing sequence.

Embodiment 46. The system of Embodiment 45, wherein the targeting sequence comprises a mismatch nucleotide opposite to the adenosine in the AG splicing sequence.

Embodiment 47. The system of Embodiment 45, wherein the targeting sequence does not comprise a mismatch nucleotide opposite to the adenosine in the AG splicing sequence.

Embodiment 48. The system of any one of Embodiments 44-47, wherein the target sequence comprises a branch point of the 5′ intron.

Embodiment 49. The system of Embodiment 48, wherein the targeting RNA sequence comprises a mismatch nucleotide opposite to the adenosine in the branch point.

Embodiment 50. The system of Embodiment 48, wherein the targeting RNA sequence does not comprise a mismatch nucleotide opposite to the adenosine in the branch point.

Embodiment 51. The system of any one of Embodiments 44-50, wherein the target sequence comprises a pyrimidine-rich sequence of the 5′ intron.

Embodiment 52. The system of any one of Embodiments 44-51, wherein the target sequence comprises an exonic splicing enhancer sequence of the target exon.

Embodiment 53. The system of any one of Embodiments 44-52, wherein the targeting RNA sequence is more than about 30 nucleotides in length.

Embodiment 54. The system of Embodiment 53, wherein the targeting RNA sequence is about 70 to about 150 nucleotides in length.

Embodiment 55. The system of any one of Embodiments 44-54, wherein the dRNA further comprises a 3′ ligation sequence and a 5′ ligation sequence.

Embodiment 56. The system of any one of Embodiments 44-55, wherein the dRNA is a circRNA.

Embodiment 57. The system of any one of Embodiments 44-55, wherein the dRNA is a linear RNA.

Embodiment 58. The system of Embodiment 57, wherein the dRNA is a linear RNA that is capable of forming a circRNA within the host cell.

Embodiment 59. The system of Embodiment 57 or 58, wherein the system comprises a construct comprising a nucleic acid encoding the dRNA.

Embodiment 60. The system of Embodiment 59, wherein the construct further comprises a 3′ twister ribozyme sequence linked to the 3′ end of the nucleic acid encoding the dRNA and a 5′ twister ribozyme sequence linked to the 5′ end of the nucleic acid encoding the dRNA.

Embodiment 61. The system of Embodiment 59 or 60, wherein the construct is a viral vector or a plasmid.

Embodiment 62. The system of Embodiment 61, wherein the construct is an AAV vector, lentiviral vector, adenoviral vector, RNA replicon, pox virus vector, enteric virus vector, Venezuelan Equine Encephalitis virus vector, Semliki Forest Virus vector, or Tobacco Mosaic Virus vector.

Embodiment 63. The system of any one of Embodiments 44-62, wherein the dRNA further comprises an ADAR recruiting domain.

Embodiment 64. The system of any one of Embodiments 44-62, wherein the dRNA does not comprises an ADAR recruiting domain.

Embodiment 65. The system of any one of Embodiments 44-64, wherein the system comprises at least two dRNAs or constructs comprising a nucleic acid encoding the dRNA, each comprising a targeting RNA sequence that is at least partially complementary to a different target sequence in the pre-mRNA.

Embodiment 66. The system of any one of Embodiments 44-65, wherein the dRNA is not chemically modified.

Embodiment 67. The system of any one of Embodiments 44-58 and 63-65, wherein the dRNA is chemically modified.

Embodiment 68. The system of any one of Embodiments 44-67, wherein the pre-mRNA is from a gene selected from the group consisting of dystrophin, TP53 gene, a MET gene, a KRAS gene, or STAT3, or a gene selected from the group consisting of RAB7A, ABCA4, SERPINA1, HEXA, LRRK2, SNCA, DMD, APP, MAPT, CFTR, ALAS1, ATP7B, HFE, LIP A, SOD1, CEP290, USH2A, CD274, HTT, COL7A1, MS4A, SGCG, DYSF, COL6A1, MTM1, LAMA2, EMD, CAPN3, ATXN3, NOTCH3, COL4A5, NF1, PCSK9 start site, or SCNN1A start site, or a fragment or any combination thereof.

Embodiment 69. The system of Embodiment 68, wherein: (1) the pre-mRNA is from a dystrophin gene, and wherein the target exon is selected from the group consisting of exons 3, 4, 5, 8, 10-16, 19-40, 42-44, 46, 47, and 50-53; (2) the pre-mRNA is from an SMN1 gene and wherein the target exon is exon 7; (3) the pre-mRNA is from a CFTR gene, and wherein the target exon is selected from the group consisting of exons 10, 11, 12, and 13; (4) the pre-mRNA is from a beta-globin gene and wherein the target exon is selected from the group consisting of exons 51, 44, 45, and 27; (5) the pre-mRNA is from a Factor VIII gene, and wherein the target exon is selected from the group consisting of exons 5, 6, 7, and 8; (6) the pre-mRNA is from a Factor IX gene, and wherein the target exon is exon 2; (7) the pre-mRNA is from a RELA gene, and wherein the target exon is exon 7; (8) the pre-mRNA is from a Trp53 genes, and wherein the target exon is selected from the group consisting of exons 2 and 5; or (9) the pre-mRNA is from a PPIB gene, and wherein the target exon is exon 3.

Embodiment 70. The system of any one of Embodiments 44-69, wherein the host cell is an eukaryotic cell.

Embodiment 71. Use of the system of any one of Embodiments 44-70 for the manufacture of a medicament for treating or preventing a disease in an individual.

Embodiment 72. The use of Embodiment 71, wherein: (1) the pre-mRNA is from a mutant dystrophin gene, and the disease or condition is Duchenne muscular dystrophy (DMD); (2) the pre-mRNA is from a mutant SMN1 gene, and the disease or condition is Spinal muscular atrophy (SMA); (3) the pre-mRNA is from a mutant CFTR gene, and the disease or condition is Cystic fibrosis (CF); (4) the pre-mRNA is from a mutant beta-globin gene, and the disease or condition is Beta-thalassemia; or (5) the pre-mRNA is from a mutant Factor VIII or Factor IX gene, and the disease or condition is hemophilia.

Embodiment 73. The use of Embodiment 71 or 72, wherein the dRNA or construct encoding the dRNA is suitable for intravenous administration.

SEQUENCES

| Annotation (SEQ ID NO:) | Sequence |
|---|---|
| arRNA targeting RELA | TGCCAGAGTTTCGGTTCACTCGGCAGATCTTGAGCTCGGCAGT |
| (SEQ ID NO: 1) | GTTGGGGGCACCcGAGGCAGTGAAAACAAGGGAGGATGACCT |
| | GAGCCACGAGAGGTCCCCAGGGTGGC |
| 5′ ligation spacer for | AACCATGCCGACTGATGGCAG |
| arRNA linker (SEQ ID | |
| NO: 2) | |
| 3′ ligation spacer for | CTGCCATCAGTCGGCGTGGACTGTAG |
| arRNA linker (SEQ ID | |
| NO: 3) | |
| arRNA targeting Trp53 | GCAGGAACTGTTACACATGTAGTTGTAGTGGATGGTGGTACAG |
| Exon 2 (SEQ ID NO: 4) | TCAGAGCCAACCcAGGAGATAACACAGGCCCAAGATGAGGCC |
| | AGTGCGCCTTGGGGAGACCTGTGGCA |
| Control RNA (SEQ ID | actacagttgctccgatatttaggctacgtcaataggcactaacttattggcgctggtgaacggact |
| NO: 5) | tcctctcgagtaccagaagatgactacaaaactcctttccattgcgagtatcggagtctggctcagt |
| | ttggccagggaggcact |
| arRNA targeting PPIB | AGTGCTTCAGTTTGAAGTTCTCATCGGGGAAGCGCTCACCGTA |
| (SEQ ID NO: 6) | GATGCTCTTTCCcGGGAAAAAAGACAGAGCAGGTCAGGGGCGC |
| | TGGATTGCGCCAAACCAAGCAGACA |
| arRNA 71 nt targeting | CGATGTCAGTACTTCCAATATTCACTAAATCAACcCGTTAAAGA |
| DMD (SEQ ID NO: 7) | GAAGGGGAAAACATTTGAAGGTAAGAA |
| arRNA 111 nt targeting | cagTTTATGATTTCCATCTACGATGTCAGTACTTCCAATATTCAC |
| DMD (SEQ ID NO: 8) | TAAATCAACcCGTTAAAGAGAAGGGGAAAACATTTGAAGGTAA |
| | GAAACCAAATGCCTAGTTGCAAT |
| arRNA 151 nt targeting | TTCCAAATCAAACCAAGAgtcagTTTATGATTTCCATCTACGATG |
| DMD (SEQ ID NO: 9) | TCAGTACTTCCAATATTCACTAAATCAACcCGTTAAAGAGAAG |
| | GGGAAAACATTTGAAGGTAAGAAACCAAATGCCTAGTTGCAA |
| | TAACAATAATAAAAATGTGAA |
| Forward primer for RELA | CCCACGAGCTTGTAGGAAAGGA |
| (SEQ ID NO: 10) | |
| Reverse primer for RELA | CCTGGTCCCGTGAAATACACCT |
| (SEQ ID NO: 11) | |
| arRNA targeting Trp53 | TTCCAAGGCCTCATTCAGCTCTCGGAACATCTCGAAGCGCTCA |
| Exon 5 (SEQ ID NO: 12) | CGCCCACGGATCcGCAGCAACAGAGGAGGGGGAGAAGTAAGT |
| | ATATACACAGTACCTGAGTTAAAAGA |
| Forward primer for Trp53 | TGGAAGGAAATTTGCGTGTGG |
| (SEQ ID NO: 13) | |
| Reverse primer for Trp53 | TATGGCGGGAGGTAGACTGA |
| (SEQ ID NO: 14) | |
| Forward primer for PPIB | GAGCCCGCGAGCAACC |
| (SEQ ID NO: 15) | |
| Reverse primer for PPIB | GGGCCTGTGGAATGTGAGGG |
| (SEQ ID NO: 16) | |
| GluA2-EIE1 (SEQ ID | CAAGGTTGGTCACTCACCTGCTTCAACTTTGTGCATTTTAGGTC |
| NO: 17) | TCAAGTGGATATTCATGGTGTCTATGAATTCACTAGAAATGGT |
| | GAAATCATATACACCATGGAGAGGCTATAAAATGCAGAAGGT |
| | TCCTATCATTCCATGCCTGCTTG |
| GluA2-EIE2 (SEQ ID | CTCCACTAAAAAAAAAACAAACGACAAAATAGGAATACAGAG |
| NO: 18) | TTTGTATTTCCTTAAAATGAATGTGTTTTAACTCTTAAAGGAA |
| Gabra-3-EIE (SEQ ID | AGGAAGGGCTGAGAAGCACTTCCTGAGAGATCTGTATTGGGAC |
| NO: 19) | CAGAAGTGAGTAGCCTGTAGCCCGCAGGAACTCAGCAGGGCT |
| | ATGTTCTATTCACTGGGGTCATCAATACAATTCTCAACTGGAGT |
| | GCTTCTTGGTAATCTGGCCT |
| FLNB-EIE (SEQ ID NO: | GGTGAGATGCAAGGAAGCATCCATCTCCTTGGCCGCGAAAGTG |
| 20) | GCCATTGAGGAGGGCTGGGTCTCTC |
| GluA2 (R/G) (SEQ ID | CTAAAGGATCCTCATTAAGGTGGGTGGAATAGTATAACAATAT |
| NO: 21) | GCTAAATGTTGTTATAGTATCCCACCTACCCTGATGTATCTTTA |
| | AG |
| GluK1-EIE1 (SEQ ID | GTGTAAACCTGTGGTAGTTAACAGAAACAGTCTGTAAATATCA |
| NO: 22) | GAGAAACACAC |
| GluK1-EIE2 (SEQ ID | GTGGTGGAAAACAATTTTACTTTACTAAATAGTTTCTGGTTTGG |
| NO: 23) | AGTTGGAGCTCTCATGCATTTCATGCTGGTAAACTCCACCATG |
| | AAGCTAGAGGGTAGGATGAAACACCAT |
| CopaE-EIE (SEQ ID NO: | GAGAAAAACTCCACGCGGTGGGCAGGGTACTTCCTGCCAACCT |
| 24) | CAAGCGTGGGCTGTACCCGGCCAGAGCAGAAGCCTACCATCCA |
| | GAATTATTTCTGCGTAGGCACTCAGCCCAGGGCCAAGACCTGC |
| | TTGCTATGGTGACTCTGGTCTGGCCGGGCCCAGGAGCCCCAGA |
| | TGGGCAGTTTCTCTC |
| 3′ splice site | TAG |
| Branchpoint | YUNAY |
| Branchpoint | CURAY |
| Branchpoint | YNCURAY |
| Branchpoint | YNCURAC |
| Branchpoint | YNYURAY |
| arRNA (SEQ ID NO: 31) | ucgccguccagcucgaccaggaugggcaccaccccggugaacagcuccucgcccuugcucacu |
| | ggcagagcccuccagcaucgcgagcaggcgcugccuccuccgccgcugccuccuccgccgcug |
| | ccuccuccgcccugcagcuuguaca |
| ADAR1 p110 QAA (SEQ | ATGGCCGAGATCAAGGAGAAAATCTGCGACTATCTCTTCAATG |
| ID NO: 32) | TGTCTGACTCCTCTGCCCTGAATTTGGCTAAAAATATTGGCCTT |
| | ACCAAGGCCCGAGATATAAATGCTGTGCTAATTGACATGGAAA |
| | GGCAGGGGGATGTCTATAGACAAGGGACAACCCCTCCCATATG |
| | GCATTTGACAGACAAGAAGCGAGAGAGGATGCAAATCAAGAG |
| | AAATACGAACAGTGTTCCTGAAACCGCTCCAGCTGCAATCCCT |
| | GAGACCAAAAGAAACGCAGAGTTCCTCACCTGTAATATACCCA |
| | CATCAAATGCCTCAAATAACATGGTAACCACAGAAAAAGTGG |
| | AGAATGGGCAGGAACCTGTCATAAAGTTAGAAAACAGGCAAG |
| | AGGCCAGACCAGAACCAGCAAGACTGAAACCACCTGTTCATTA |
| | CAATGGCCCCTCAAAAGCAGGGTATGTTGACTTTGAAAATGGC |
| | CAGTGGGCCACAGATGACATCCCAGATGACTTGAATAGTATCC |
| | GCGCAGCACCAGGTGAGTTTCGAGCCATCATGGAGATGCCCTC |
| | CTTCTACAGTCATGGCTTGCCACGGTGTTCACCCTACAAGAAA |
| | CTGACAGAGTGCCAGCTGAAGAACCCCATCAGCGGGCTGTTAG |
| | AATATGCCCAGTTCGCTAGTCAAACCTGTGAGTTCAACATGAT |
| | AGAGCAGAGTGGACCACCCCATGAACCTCGATTTAAATTCCAG |
| | GTTGTCATCAATGGCCGAGAGTTTCCCCCAGCTGAAGCTGGAA |
| | GCAAGAAAGTGGCCAAGCAGGATGCAGCTATGAAAGCCATGA |
| | CAATTCTGCTAGAGGAAGCCAAAGCCAAGGACAGTGGAAAAT |
| | CAGAAGAATCATCCCACTATTCCACAGAGAAAGAATCAGAGA |
| | AGACTGCAGAGTCCCAGACCCCCACCCCTTCAGCCACATCCTT |
| | CTTTTCTGGGAAGAGCCCCGTCACCACACTGCTTGAGTGTATG |
| | CACAAATTGGGGAACTCCTGCGAATTCCGTCTCCTGTCCAAAG |
| | AAGGCCCTGCCCATGAACCCAAGTTCCAATACTGTGTTGCAGT |
| | GGGAGCCCAAACTTTCCCCAGTGTGAGTGCTCCCAGCAAGAAA |
| | GTGGCAAAGCAGATGGCCGCAGAGGAAGCCATGAAGGCCCTG |
| | CATGGGGAGGCGACCAACTCCATGGCTTCTGATAACCAGCCTG |
| | AAGGTATGATCTCAGAGTCACTTGATAACTTGGAATCCATGAT |
| | GCCCAACAAGGTCAGGAAGATTGGCGAGCTCGTGAGATACCT |
| | GAACACCAACCCTGTGGGTGGCCTTTTGGAGTACGCCCGCTCC |
| | CATGGCTTTGCTGCTGAATTCAAGTTGGTCGACCAGTCCGGAC |
| | CTCCTCACGAGCCCAAGTTCGTTTACCAAGCAAAAGTTGGGGG |
| | TCGCTGGTTCCCAGCCGTCTGCGCACACAGCAAGAAGCAAGGC |
| | AAGCAGGAAGCAGCAGATGCGGCTCTCCGTGTCTTGATTGGGG |
| | AGAACGAGAAGGCAGAACGCATGGGTTTCACAGAGGTAACCC |
| | CAGTGACAGGGGCCAGTCTCAGAAGAACTATGCTCCTCCTCTC |
| | AAGGTCCCCAGAAGCACAGCCAAAGACACTCCCTCTCACTGGC |
| | AGCACCTTCCATGACCAGATAGCCATGCTGAGCCACCGGTGCT |
| | TCAACACTCTGACTAACAGCTTCCAGCCCTCCTTGCTCGGCCGC |
| | AAGATTCTGGCCGCCATCATTATGAAAAAAGACTCTGAGGACA |
| | TGGGTGTCGTCGTCAGCTTGGGAACAGGGAATCGCTGTGTAAA |
| | AGGAGATTCTCTCAGCCTAAAAGGAGAAACTGTCAATGACTGC |
| | CAAGCTGCAATCATCTCCCGGAGAGGCTTCATCAGGTTTCTCT |
| | ACAGTGAGTTAATGAAATACAACTCCCAGACTGCGAAGGATA |
| | GTATATTTGAACCTGCTAAGGGAGGAGAAAAGCTCCAAATAA |
| | AAAAGACTGTGTCATTCCATCTGTATATCAGCACTGCTCCGTGT |
| | GGAGATGGCGCCCTCTTTGACAAGTCCTGCAGCGACCGTGCTA |
| | TGGAAAGCACAGAATCCCGCCACTACCCTGTCTTCGAGAATCC |
| | CAAACAAGGAAAGCTCCGCACCAAGGTGGAGAACGGAGAAGG |
| | CACAATCCCTGTGGAATCCAGTGACATTGTGCCTACGTGGGAT |
| | GGCATTCGGCTCGGGGAGAGACTCCGTACCATGTCCTGTAGTG |
| | ACAAAATCCTACGCTGGAACGTGCTGGGCCTGCAAGGGGCACT |
| | GTTGACCCACTTCCTGCAGCCCATTTATCTCAAATCTGTCACAT |
| | TGGGTTACCTTTTCAGCCAAGGGCATCTGACCCGTGCTATTTGC |
| | TGTCGTGTGACAAGAGATGGGAGTGCATTTGAGGATGGACTAC |
| | GACATCCCTTTATTGTCAACCACCCCAAGGTTGGCAGAGTCAG |
| | CATATATGATTCCAAAAGGCAATCCGGGAAGACTAAGGAGAC |
| | AAGCGTCAACTGGTGTCTGGCTGATGGCTATGACCTGGAGATC |
| | CTGGACGGTACCAGAGGCACTGTGGATGGGCCACGGAATGAA |
| | TTGTCCCGGGTCTCCAAAAAGAACATTTTTCTTCTATTTAAGAA |
| | GCTCTGCTCCTTCCGTTACCGCAGGGATCTACTGAGACTCTCCT |
| | ATGGTGAGGCCAAGAAAGCTGCCCGTGACTACGAGACGGCCA |
| | AGAACTACTTCAAAAAAGGCCTGAAGGATATGGGCTATGGGA |
| | ACTGGATTAGCAAACCCCAGGAGGAAAAGAACTTTTATCTCTG |
| | CCCAGTA |
| | ATGGCCGAGATCAAGGAGAAAATCTGCGACTATCTCTTCAATG |
| | TGTCTGACTCCTCTGCCCTGAATTTGGCTAAAAATATTGGCCTT |
| | ACCAAGGCCCGAGATATAAATGCTGTGCTAATTGACATGGAAA |
| | GGCAGGGGGATGTCTATAGACAAGGGACAACCCCTCCCATATG |
| | GCATTTGACAGACAAGAAGCGAGAGAGGATGCAAATCAAGAG |
| | AAATACGAACAGTGTTCCTGAAACCGCTCCAGCTGCAATCCCT |
| ADAR1 p110 HAG (SEQ | GAGACCAAAAGAAACGCAGAGTTCCTCACCTGTAATATACCCA |
| ID NO: 33) | CATCAAATGCCTCAAATAACATGGTAACCACAGAAAAAGTGG |
| | AGAATGGGCAGGAACCTGTCATAAAGTTAGAAAACAGGCAAG |
| | AGGCCAGACCAGAACCAGCAAGACTGAAACCACCTGTTCATTA |
| | CAATGGCCCCTCAAAAGCAGGGTATGTTGACTTTGAAAATGGC |
| | CAGTGGGCCACAGATGACATCCCAGATGACTTGAATAGTATCC |
| | GCGCAGCACCAGGTGAGTTTCGAGCCATCATGGAGATGCCCTC |
| | CTTCTACAGTCATGGCTTGCCACGGTGTTCACCCTACAAGAAA |
| | CTGACAGAGTGCCAGCTGAAGAACCCCATCAGCGGGCTGTTAG |
| | AATATGCCCAGTTCGCTAGTCAAACCTGTGAGTTCAACATGAT |
| | AGAGCAGAGTGGACCACCCCATGAACCTCGATTTAAATTCCAG |
| | GTTGTCATCAATGGCCGAGAGTTTCCCCCAGCTGAAGCTGGAA |
| | GCAAGAAAGTGGCCAAGCAGGATGCAGCTATGAAAGCCATGA |
| | CAATTCTGCTAGAGGAAGCCAAAGCCAAGGACAGTGGAAAAT |
| | CAGAAGAATCATCCCACTATTCCACAGAGAAAGAATCAGAGA |
| | AGACTGCAGAGTCCCAGACCCCCACCCCTTCAGCCACATCCTT |
| | CTTTTCTGGGAAGAGCCCCGTCACCACACTGCTTGAGTGTATG |
| | CACAAATTGGGGAACTCCTGCGAATTCCGTCTCCTGTCCAAAG |
| | AAGGCCCTGCCCATGAACCCAAGTTCCAATACTGTGTTGCAGT |
| | GGGAGCCCAAACTTTCCCCAGTGTGAGTGCTCCCAGCAAGAAA |
| | GTGGCAAAGCAGATGGCCGCAGAGGAAGCCATGAAGGCCCTG |
| | CATGGGGAGGCGACCAACTCCATGGCTTCTGATAACCAGCCTG |
| | AAGGTATGATCTCAGAGTCACTTGATAACTTGGAATCCATGAT |
| | GCCCAACAAGGTCAGGAAGATTGGCGAGCTCGTGAGATACCT |
| | GAACACCAACCCTGTGGGTGGCCTTTTGGAGTACGCCCGCTCC |
| | CATGGCTTTGCTGCTGAATTCAAGTTGGTCGACCAGTCCGGAC |
| | CTCCTCACGAGCCCAAGTTCGTTTACCAAGCAAAAGTTGGGGG |
| | TCGCTGGTTCCCAGCCGTCTGCGCACACAGCAAGAAGCAAGGC |
| | AAGCAGGAAGCAGCAGATGCGGCTCTCCGTGTCTTGATTGGGG |
| | AGAACGAGAAGGCAGAACGCATGGGTTTCACAGAGGTAACCC |
| | CAGTGACAGGGGCCAGTCTCAGAAGAACTATGCTCCTCCTCTC |
| | AAGGTCCCCAGAAGCACAGCCAAAGACACTCCCTCTCACTGGC |
| | AGCACCTTCCATGACCAGATAGCCATGCTGAGCCACCGGTGCT |
| | TCAACACTCTGACTAACAGCTTCCAGCCCTCCTTGCTCGGCCGC |
| | AAGATTCTGGCCGCCATCATTATGAAAAAAGACTCTGAGGACA |
| | TGGGTGTCGTCGTCAGCTTGGGAACAGGGAATCGCTGTGTAAA |
| | AGGAGATTCTCTCAGCCTAAAAGGAGAAACTGTCAATGACTGC |
| | CATGCCGGCATCATCTCCCGGAGAGGCTTCATCAGGTTTCTCTA |
| | CAGTGAGTTAATGAAATACAACTCCCAGACTGCGAAGGATAGT |
| | ATATTTGAACCTGCTAAGGGAGGAGAAAAGCTCCAAATAAAA |
| | AAGACTGTGTCATTCCATCTGTATATCAGCACTGCTCCGTGTGG |
| | AGATGGCGCCCTCTTTGACAAGTCCTGCAGCGACCGTGCTATG |
| | GAAAGCACAGAATCCCGCCACTACCCTGTCTTCGAGAATCCCA |
| | AACAAGGAAAGCTCCGCACCAAGGTGGAGAACGGAGAAGGCA |
| | CAATCCCTGTGGAATCCAGTGACATTGTGCCTACGTGGGATGG |
| | CATTCGGCTCGGGGAGAGACTCCGTACCATGTCCTGTAGTGAC |
| | AAAATCCTACGCTGGAACGTGCTGGGCCTGCAAGGGGCACTGT |
| | TGACCCACTTCCTGCAGCCCATTTATCTCAAATCTGTCACATTG |
| | GGTTACCTTTTCAGCCAAGGGCATCTGACCCGTGCTATTTGCTG |
| | TCGTGTGACAAGAGATGGGAGTGCATTTGAGGATGGACTACGA |
| | CATCCCTTTATTGTCAACCACCCCAAGGTTGGCAGAGTCAGCA |
| | TATATGATTCCAAAAGGCAATCCGGGAAGACTAAGGAGACAA |
| | GCGTCAACTGGTGTCTGGCTGATGGCTATGACCTGGAGATCCT |
| | GGACGGTACCAGAGGCACTGTGGATGGGCCACGGAATGAATT |
| | GTCCCGGGTCTCCAAAAAGAACATTTTTCTTCTATTTAAGAAGC |
| | TCTGCTCCTTCCGTTACCGCAGGGATCTACTGAGACTCTCCTAT |
| | GGTGAGGCCAAGAAAGCTGCCCGTGACTACGAGACGGCCAAG |
| | AACTACTTCAAAAAAGGCCTGAAGGATATGGGCTATGGGAACT |
| | GGATTAGCAAACCCCAGGAGGAAAAGAACTTTTATCTCTGCCC |
| | AGTA |
| ADAR1 p110 HAE (active | ATGGCCGAGATCAAGGAGAAAATCTGCGACTATCTCTTCAATG |
| form) (SEQ ID NO: 34) | TGTCTGACTCCTCTGCCCTGAATTTGGCTAAAAATATTGGCCTT |
| | ACCAAGGCCCGAGATATAAATGCTGTGCTAATTGACATGGAAA |
| | GGCAGGGGGATGTCTATAGACAAGGGACAACCCCTCCCATATG |
| | GCATTTGACAGACAAGAAGCGAGAGAGGATGCAAATCAAGAG |
| | AAATACGAACAGTGTTCCTGAAACCGCTCCAGCTGCAATCCCT |
| | GAGACCAAAAGAAACGCAGAGTTCCTCACCTGTAATATACCCA |
| | CATCAAATGCCTCAAATAACATGGTAACCACAGAAAAAGTGG |
| | AGAATGGGCAGGAACCTGTCATAAAGTTAGAAAACAGGCAAG |
| | AGGCCAGACCAGAACCAGCAAGACTGAAACCACCTGTTCATTA |
| | CAATGGCCCCTCAAAAGCAGGGTATGTTGACTTTGAAAATGGC |
| | CAGTGGGCCACAGATGACATCCCAGATGACTTGAATAGTATCC |
| | GCGCAGCACCAGGTGAGTTTCGAGCCATCATGGAGATGCCCTC |
| | CTTCTACAGTCATGGCTTGCCACGGTGTTCACCCTACAAGAAA |
| | CTGACAGAGTGCCAGCTGAAGAACCCCATCAGCGGGCTGTTAG |
| | AATATGCCCAGTTCGCTAGTCAAACCTGTGAGTTCAACATGAT |
| | AGAGCAGAGTGGACCACCCCATGAACCTCGATTTAAATTCCAG |
| | GTTGTCATCAATGGCCGAGAGTTTCCCCCAGCTGAAGCTGGAA |
| | GCAAGAAAGTGGCCAAGCAGGATGCAGCTATGAAAGCCATGA |
| | CAATTCTGCTAGAGGAAGCCAAAGCCAAGGACAGTGGAAAAT |
| | CAGAAGAATCATCCCACTATTCCACAGAGAAAGAATCAGAGA |
| | AGACTGCAGAGTCCCAGACCCCCACCCCTTCAGCCACATCCTT |
| | CTTTTCTGGGAAGAGCCCCGTCACCACACTGCTTGAGTGTATG |
| | CACAAATTGGGGAACTCCTGCGAATTCCGTCTCCTGTCCAAAG |
| | AAGGCCCTGCCCATGAACCCAAGTTCCAATACTGTGTTGCAGT |
| | GGGAGCCCAAACTTTCCCCAGTGTGAGTGCTCCCAGCAAGAAA |
| | GTGGCAAAGCAGATGGCCGCAGAGGAAGCCATGAAGGCCCTG |
| | CATGGGGAGGCGACCAACTCCATGGCTTCTGATAACCAGCCTG |
| | AAGGTATGATCTCAGAGTCACTTGATAACTTGGAATCCATGAT |
| | GCCCAACAAGGTCAGGAAGATTGGCGAGCTCGTGAGATACCT |
| | GAACACCAACCCTGTGGGTGGCCTTTTGGAGTACGCCCGCTCC |
| | CATGGCTTTGCTGCTGAATTCAAGTTGGTCGACCAGTCCGGAC |
| | CTCCTCACGAGCCCAAGTTCGTTTACCAAGCAAAAGTTGGGGG |
| | TCGCTGGTTCCCAGCCGTCTGCGCACACAGCAAGAAGCAAGGC |
| | AAGCAGGAAGCAGCAGATGCGGCTCTCCGTGTCTTGATTGGGG |
| | AGAACGAGAAGGCAGAACGCATGGGTTTCACAGAGGTAACCC |
| | CAGTGACAGGGGCCAGTCTCAGAAGAACTATGCTCCTCCTCTC |
| | AAGGTCCCCAGAAGCACAGCCAAAGACACTCCCTCTCACTGGC |
| | AGCACCTTCCATGACCAGATAGCCATGCTGAGCCACCGGTGCT |
| | TCAACACTCTGACTAACAGCTTCCAGCCCTCCTTGCTCGGCCGC |
| | AAGATTCTGGCCGCCATCATTATGAAAAAAGACTCTGAGGACA |
| | TGGGTGTCGTCGTCAGCTTGGGAACAGGGAATCGCTGTGTAAA |
| | AGGAGATTCTCTCAGCCTAAAAGGAGAAACTGTCAATGACTGC |
| | CATGCAGAAATAATCTCCCGGAGAGGCTTCATCAGGTTTCTCT |
| | ACAGTGAGTTAATGAAATACAACTCCCAGACTGCGAAGGATA |
| | GTATATTTGAACCTGCTAAGGGAGGAGAAAAGCTCCAAATAA |
| | AAAAGACTGTGTCATTCCATCTGTATATCAGCACTGCTCCGTGT |
| | GGAGATGGCGCCCTCTTTGACAAGTCCTGCAGCGACCGTGCTA |
| | TGGAAAGCACAGAATCCCGCCACTACCCTGTCTTCGAGAATCC |
| | CAAACAAGGAAAGCTCCGCACCAAGGTGGAGAACGGAGAAGG |
| | CACAATCCCTGTGGAATCCAGTGACATTGTGCCTACGTGGGAT |
| | GGCATTCGGCTCGGGGAGAGACTCCGTACCATGTCCTGTAGTG |
| | ACAAAATCCTACGCTGGAACGTGCTGGGCCTGCAAGGGGCACT |
| | GTTGACCCACTTCCTGCAGCCCATTTATCTCAAATCTGTCACAT |
| | TGGGTTACCTTTTCAGCCAAGGGCATCTGACCCGTGCTATTTGC |
| | TGTCGTGTGACAAGAGATGGGAGTGCATTTGAGGATGGACTAC |
| | GACATCCCTTTATTGTCAACCACCCCAAGGTTGGCAGAGTCAG |
| | CATATATGATTCCAAAAGGCAATCCGGGAAGACTAAGGAGAC |
| | AAGCGTCAACTGGTGTCTGGCTGATGGCTATGACCTGGAGATC |
| | CTGGACGGTACCAGAGGCACTGTGGATGGGCCACGGAATGAA |
| | TTGTCCCGGGTCTCCAAAAAGAACATTTTTCTTCTATTTAAGAA |
| | GCTCTGCTCCTTCCGTTACCGCAGGGATCTACTGAGACTCTCCT |
| | ATGGTGAGGCCAAGAAAGCTGCCCGTGACTACGAGACGGCCA |
| | AGAACTACTTCAAAAAAGGCCTGAAGGATATGGGCTATGGGA |
| | ACTGGATTAGCAAACCCCAGGAGGAAAAGAACTTTTATCTCTG |
| | CCCAGTA |
| 151-SA (SEQ ID NO: 35) | CAUUUCUAGUUUGGAGAUGACAGUUUCCUUAGUAACCACAG |
| | AUUGUGUCACUAGAGUAACAGUCUGACUGGCAGCUAGUGUU |
| | UUUGAAAGAAAGAAGAAAAGAAAAAAAAAAGGGAAUAUGAG |
| | AAACACAAGCAAAAGAGCCAAUUUUAUA |
| 151-1 (SEQ ID NO: 36) | ACCCACCAUCACUCUCUGUGAUUUUAUAACUCGAUCAAGCAG |
| | AGACAGCCAGUCUGUAAGUUCUGUCCAAGCUCGGUUGAAGU |
| | CUGCCAGUGCAGGUACCUCCAACAGCAAAGAAGAUGGCAUU |
| | UCUAGUUUGGAGAUGACAGUUUCCUUA |
| 151-2 (SEQ ID NO: 37) | GCAGAGACAGCCAGUCUGUAAGUUCUGUCCAAGCUCGGUUG |
| | AAGUCUGCCAGUGCAGGUACCUCCAACAGCAAAGAAGAUGG |
| | CAUUUCUAGUUUGGAGAUGACAGUUUCCUUAGUAACCACAG |
| | AUUGUGUCACUAGAGUAACAGUCUGACU |
| 151-3 (SEQ ID NO: 38) | CAGUCUGUAAGUUCUGUCCAAGCUCGGUUGAAGUCUGCCAG |
| | UGCAGGUACCUCCAACAGCAAAGAAGAUGGCAUUUCUAGUU |
| | UGGAGAUGACAGUUUCCUUAGUAACCACAGAUUGUGUCACU |
| | AGAGUAACAGUCUGACUGGCAGCUAGUG |
| 71-1 (SEQ ID NO: 39) | UUCUAGUUUGGAGAUGACAGUUUCCUUAGUAACCACAGAUU |
| | GUGUCACUAGAGUAACAGUCUGACUGGCAG |
| 71-2 (SEQ ID NO: 40) | CAAGCAGAGACAGCCAGUCUGUAAGUUCUGUCCAAGCUCGG |
| | UUGAAGUCUGCCAGUGCAGGUACCUCCAAC |
| 31 (SEQ ID NO: 41) | UCCAAGCUCGGUUGAAGUCUGCCAGUGCAGG |
| 39 (SEQ ID NO: 42) | GUUCUGUCCAAGCUCGGUUGAAGUCUGCCAGUGCAGGUA |
| 47 (SEQ ID NO: 43) | GUCUGUAAGUUCUGUCCAAGCUCGGUUGAAGUCUGCCAGUG |
| | CAGGUA |
| 55 (SEQ ID NO: 44) | GACAGCCAGUCUGUAAGUUCUGUCCAAGCUCGGUUGAAGUC |
| | UGCCAGUGCAGGUA |
| 63 (SEQ ID NO: 45) | GACAGCCAGUCUGUAAGUUCUGUCCAAGCUCGGUUGAAGUC |
| | UGCCAGUGCAGGUACCUCCAAC |
| STAT3 arRNA (SEQ ID | GAAUCUAAAGUGCGGGGGGACAUCGGCAGGUCAAUGGUAUU |
| NO: 46) | GCUGCAGGUCGUUCcGUAGGAAAUGGGGGGCAGCAGGAGGG |
| | GAAACGGGGGGUUGACAAGACACAAUGGA |

1. A method of modulating splicing of a target exon in a pre-mRNA in a host cell, the pre-mRNA comprising the target exon flanked by a 5′ intron and a 3′ intron, wherein the method comprises introducing a deaminase-recruiting RNA (dRNA) or a construct comprising a nucleic acid encoding the dRNA into the host cell, wherein:
(1) dRNA comprises a targeting RNA sequence that is at least partially complementary to a target sequence in the pre-mRNA comprising a portion of the target exon, a portion of the 5′ intron, and/or a portion of the 3′ intron; and
(2) the dRNA is capable of recruiting an adenosine deaminase acting on RNA (ADAR) to the target exon, the 5′ intron, and/or the 3′ intron.

2. The method of claim 1, wherein the recruitment of the ADAR blocks access of one or more splicing factors to a splicing site for the target exon, thereby modulating the splicing of the target exon in the pre-mRNA.

3. The method of claim 1, wherein the recruitment of the ADAR does not deaminate a target adenosine in the 5′ intron or the 3′ intron.

4. The method of claim 1, wherein the recruitment of the ADAR deaminates a target adenosine in the 5′ intron or the 3′ intron, thereby modulating the splicing of the target exon in the pre-mRNA.

5. The method of claim 1, wherein the pre-mRNA is spliced in the host cell to form a mature mRNA not including the target exon.

6. (canceled)

7. A system for modulating splicing of a target exon in a pre-mRNA in a host cell, the pre-mRNA comprising the target exon flanked by a 5′ intron and a 3′ intron, wherein the system comprises a dRNA or a construct comprising a nucleic acid encoding the dRNA, wherein:
(1) the dRNA comprises a targeting RNA sequence that is at least partially complementary to a target sequence in the pre-mRNA comprising a portion of the target exon, a portion of the 5′ intron, and/or a portion of the 3′ intron; and
(2) the dRNA is capable of recruiting an adenosine deaminase acting on RNA (ADAR) to the target exon, the 5′ intron, and/or the 3′ intron.

8. The method of claim 1, wherein the target sequence comprises a 3′ splice site of the 5′ intron comprising an AG splicing sequence.

9. The method of claim 8, wherein the targeting RNA sequence comprises a mismatch nucleotide opposite to the adenosine in the AG splicing sequence, thereby inducing deamination at the adenosine upon recruitment of the ADAR.

10. (canceled)

11. The method of claim 1, wherein the target sequence comprises a branch point of the 5′ intron.

12. The method of claim 11, wherein the targeting RNA sequence comprises a mismatch nucleotide opposite to the adenosine in the branch point, thereby inducing deamination at the adenosine upon recruitment of the ADAR.

13. (canceled)

14. The method of claim 1, wherein the target sequence comprises a pyrimidine-rich sequence of the 5′ intron, or the target sequence comprises an exonic splicing enhancer sequence of the target exon.

15. The method of claim 1, wherein the targeting RNA sequence is more than about 30 nucleotides in length.

16. The method of claim 1, wherein the dRNA further comprises a 3′ ligation sequence and a 5′ ligation sequence.

17. The method of claim 1, wherein the dRNA is a circRNA.

18. The method of claim 1, wherein the dRNA is a linear RNA.

19-22. (canceled)

23. The method of claim 1, wherein the dRNA does not comprises an ADAR recruiting domain.

24. The method of claim 1, wherein the dRNA is not chemically modified.

25. (canceled)

26. The method of claim 1, wherein the pre-mRNA is from a gene selected from the group consisting of dystrophin, RELA, Trp53, PPIB, STAT3, SMN1, CFTR, beta-globin, Factor VIII, Factor IX, TP53, mesenchymal epithelial transition factor receptor (MET), KRAS, RAB7A, ABCA4, SERPINA1, HEXA, LRRK2, SNCA, DMD, APP, MAPT, CFTR, ALAS1, ATP7B, HFE, LIP A, CEP290, USH2A, CD274, HTT, COL7A1, MS4A, SGCG, ATXN3, NOTCH3, COL4A5, NF1, PCSK9 start site, SCNN1A, a collagen VI alpha 1 gene (COL6A1), a myotubular myopathy 1 gene (MTM1), a dysferlin gene (DYSF), a laminin-alpha 2 gene (LAMA2), an emery-dreyfuss muscular dystrophy gene (EMD), a calpain 3 gene (CAPN3), and superoxide dismutase 1 (SOD1).

27. (canceled)

28. A method of treating or preventing a disease of an individual, comprising inducing exon skipping in a pre-mRNA of a host cell in the individual according to the method of claim 1.

29-30. (canceled)

31. A ribonucleic acid (RNA) molecule, comprising a targeting RNA sequence complementary to a target RNA sequence, wherein the target RNA sequence comprises at least one of the group consisting of a 5′ splice site (5′ ss), a polypyrimidine tract, a branchpoint, and a 3′ splice site (3′ ss) of a pre-messenger RNA (pre-mRNA), and the RNA molecule is capable of recruiting an adenosine deaminase acting on RNA (ADAR) to the target RNA sequence.

32-40. (canceled)


SEQUENCE LISTING (46 sequences)

SEQ ID NO: 1 - RNA, 111 nt, synthetic construct
  source 1..111: mol_type = other RNA
tgccagagtttcggttcactcggcagatcttgagctcggcagtgttgggggcacccgaggcagtgaaaacaagggaggatgacctgagccacgagaggtccccagggtggc

SEQ ID NO: 2 - DNA, 21 bp, synthetic construct
  source 1..21: mol_type = other DNA
aaccatgccgactgatggcag

SEQ ID NO: 3 - DNA, 26 bp, synthetic construct
  source 1..26: mol_type = other DNA
ctgccatcagtcggcgtggactgtag

SEQ ID NO: 4 - DNA, 111 bp, synthetic construct
  source 1..111: mol_type = other DNA
gcaggaactgttacacatgtagttgtagtggatggtggtacagtcagagccaacccaggagataacacaggcccaagatgaggccagtgcgccttggggagacctgtggca

SEQ ID NO: 5 - RNA, 151 nt, synthetic construct
  source 1..151: mol_type = other RNA
actacagttgctccgatatttaggctacgtcaataggcactaacttattggcgctggtgaacggacttcctctcgagtaccagaagatgactacaaaactcctttccattgcgagtatcggagtctggctcagtttggccagggaggcact

SEQ ID NO: 6 - RNA, 111 nt, synthetic construct
  source 1..111: mol_type = other RNA
agtgcttcagtttgaagttctcatcggggaagcgctcaccgtagatgctctttcccgggaaaaaagacagagcaggtcaggggcgctggattgcgccaaaccaagcagaca

SEQ ID NO: 7 - RNA, 71 nt, synthetic construct
  source 1..71: mol_type = other RNA
cgatgtcagtacttccaatattcactaaatcaacccgttaaagagaaggggaaaacatttgaaggtaagaa

SEQ ID NO: 8 - RNA, 111 nt, synthetic construct
  source 1..111: mol_type = other RNA
cagtttatgatttccatctacgatgtcagtacttccaatattcactaaatcaacccgttaaagagaaggggaaaacatttgaaggtaagaaaccaaatgcctagttgcaat

SEQ ID NO: 9 - RNA, 151 nt, synthetic construct
  source 1..151: mol_type = other RNA
ttccaaatcaaaccaagagtcagtttatgatttccatctacgatgtcagtacttccaatattcactaaatcaacccgttaaagagaaggggaaaacatttgaaggtaagaaaccaaatgcctagttgcaataacaataataaaaatgtgaa

SEQ ID NO: 10 - DNA, 22 bp, synthetic construct
  source 1..22: mol_type = other DNA
cccacgagcttgtaggaaagga

SEQ ID NO: 11 - DNA, 22 bp, synthetic construct
  source 1..22: mol_type = other DNA
cctggtcccgtgaaatacacct

SEQ ID NO: 12 - DNA, 111 bp, synthetic construct
  source 1..111: mol_type = other DNA
ttccaaggcctcattcagctctcggaacatctcgaagcgctcacgcccacggatccgcagcaacagaggagggggagaagtaagtatatacacagtacctgagttaaaaga

SEQ ID NO: 13 - DNA, 21 bp, synthetic construct
  source 1..21: mol_type = other DNA
tggaaggaaatttgcgtgtgg

SEQ ID NO: 14 - DNA, 20 bp, synthetic construct
  source 1..20: mol_type = other DNA
tatggcgggaggtagactga

SEQ ID NO: 15 - DNA, 16 bp, synthetic construct
  source 1..16: mol_type = other DNA
gagcccgcgagcaacc

SEQ ID NO: 16 - DNA, 20 bp, synthetic construct
  source 1..20: mol_type = other DNA
gggcctgtggaatgtgaggg

SEQ ID NO: 17 - DNA, 152 bp, synthetic construct
  source 1..152: mol_type = other DNA
caaggttggtcactcacctgcttcaactttgtgcattttaggtctcaagtggatattcatggtgtctatgaattcactagaaatggtgaaatcatatacaccatggagaggctataaaatgcagaaggttcctatcattccatgcctgcttg

SEQ ID NO: 18 - DNA, 85 bp, synthetic construct
  source 1..85: mol_type = other DNA
ctccactaaaaaaaaaacaaacgacaaaataggaatacagagtttgtatttccttaaaatgaatgtgttttaactcttaaaggaa

SEQ ID NO: 19 - DNA, 149 bp, synthetic construct
  source 1..149: mol_type = other DNA
aggaagggctgagaagcacttcctgagagatctgtattgggaccagaagtgagtagcctgtagcccgcaggaactcagcagggctatgttctattcactggggtcatcaatacaattctcaactggagtgcttcttggtaatctggcct

SEQ ID NO: 20 - DNA, 68 bp, synthetic construct
  source 1..68: mol_type = other DNA
ggtgagatgcaaggaagcatccatctccttggccgcgaaagtggccattgaggagggctgggtctctc

SEQ ID NO: 21 - DNA, 89 bp, synthetic construct
  source 1..89: mol_type = other DNA
ctaaaggatcctcattaaggtgggtggaatagtataacaatatgctaaatgttgttatagtatcccacctaccctgatgtatctttaag

SEQ ID NO: 22 - DNA, 54 bp, synthetic construct
  source 1..54: mol_type = other DNA
gtgtaaacctgtggtagttaacagaaacagtctgtaaatatcagagaaacacac

SEQ ID NO: 23 - DNA, 114 bp, synthetic construct
  source 1..114: mol_type = other DNA
gtggtggaaaacaattttactttactaaatagtttctggtttggagttggagctctcatgcatttcatgctggtaaactccaccatgaagctagagggtaggatgaaacaccat

SEQ ID NO: 24 - DNA, 187 bp, synthetic construct
  source 1..187: mol_type = other DNA
gagaaaaactccacgcggtgggcagggtacttcctgccaacctcaagcgtgggctgtacccggccagagcagaagcctaccatccagaattatttctgcgtaggcactcagcccagggccaagacctgcttgctatggtgactctggtctggccgggcccaggagccccagatgggcagtttctctc

SEQ ID NO: 25-30 - skipped (6 entries)

SEQ ID NO: 31 - RNA, 151 nt, synthetic construct
  source 1..151: mol_type = other RNA
tcgccgtccagctcgaccaggatgggcaccaccccggtgaacagctcctcgcccttgctcactggcagagccctccagcatcgcgagcaggcgctgcctcctccgccgctgcctcctccgccgctgcctcctccgccctgcagcttgtaca

SEQ ID NO: 32 - DNA, 2793 bp, synthetic construct
  source 1..2793: mol_type = other DNA
atggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtaaaaggagattctctcagcctaaaaggagaaactgtcaatgactgccaagctgcaatcatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagta

SEQ ID NO: 33 - DNA, 2793 bp, synthetic construct
  source 1..2793: mol_type = other DNA
atggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtaaaaggagattctctcagcctaaaaggagaaactgtcaatgactgccatgccggcatcatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagta

SEQ ID NO: 34 - DNA, 2793 bp, synthetic construct
  source 1..2793: mol_type = other DNA
atggccgagatcaaggagaaaatctgcgactatctcttcaatgtgtctgactcctctgccctgaatttggctaaaaatattggccttaccaaggcccgagatataaatgctgtgctaattgacatggaaaggcagggggatgtctatagacaagggacaacccctcccatatggcatttgacagacaagaagcgagagaggatgcaaatcaagagaaatacgaacagtgttcctgaaaccgctccagctgcaatccctgagaccaaaagaaacgcagagttcctcacctgtaatatacccacatcaaatgcctcaaataacatggtaaccacagaaaaagtggagaatgggcaggaacctgtcataaagttagaaaacaggcaagaggccagaccagaaccagcaagactgaaaccacctgttcattacaatggcccctcaaaagcagggtatgttgactttgaaaatggccagtgggccacagatgacatcccagatgacttgaatagtatccgcgcagcaccaggtgagtttcgagccatcatggagatgccctccttctacagtcatggcttgccacggtgttcaccctacaagaaactgacagagtgccagctgaagaaccccatcagcgggctgttagaatatgcccagttcgctagtcaaacctgtgagttcaacatgatagagcagagtggaccaccccatgaacctcgatttaaattccaggttgtcatcaatggccgagagtttcccccagctgaagctggaagcaagaaagtggccaagcaggatgcagctatgaaagccatgacaattctgctagaggaagccaaagccaaggacagtggaaaatcagaagaatcatcccactattccacagagaaagaatcagagaagactgcagagtcccagacccccaccccttcagccacatccttcttttctgggaagagccccgtcaccacactgcttgagtgtatgcacaaattggggaactcctgcgaattccgtctcctgtccaaagaaggccctgcccatgaacccaagttccaatactgtgttgcagtgggagcccaaactttccccagtgtgagtgctcccagcaagaaagtggcaaagcagatggccgcagaggaagccatgaaggccctgcatggggaggcgaccaactccatggcttctgataaccagcctgaaggtatgatctcagagtcacttgataacttggaatccatgatgcccaacaaggtcaggaagattggcgagctcgtgagatacctgaacaccaaccctgtgggtggccttttggagtacgcccgctcccatggctttgctgctgaattcaagttggtcgaccagtccggacctcctcacgagcccaagttcgtttaccaagcaaaagttgggggtcgctggttcccagccgtctgcgcacacagcaagaagcaaggcaagcaggaagcagcagatgcggctctccgtgtcttgattggggagaacgagaaggcagaacgcatgggtttcacagaggtaaccccagtgacaggggccagtctcagaagaactatgctcctcctctcaaggtccccagaagcacagccaaagacactccctctcactggcagcaccttccatgaccagatagccatgctgagccaccggtgcttcaacactctgactaacagcttccagccctccttgctcggccgcaagattctggccgccatcattatgaaaaaagactctgaggacatgggtgtcgtcgtcagcttgggaacagggaatcgctgtgtaaaaggagattctctcagcctaaaaggagaaactgtcaatgactgccatgcagaaataatctcccggagaggcttcatcaggtttctctacagtgagttaatgaaatacaactcccagactgcgaaggatagtatatttgaacctgctaagggaggagaaaagctccaaataaaaaagactgtgtcattccatctgtatatcagcactgctccgtgtggagatggcgccctctttgacaagtcctgcagcgaccgtgctatggaaagcacagaatcccgccactaccctgtcttcgagaatcccaaacaaggaaagctccgcaccaaggtggagaacggagaaggcacaatccctgtggaatccagtgacattgtgcctacgtgggatggcattcggctcggggagagactccgtaccatgtcctgtagtgacaaaatcctacgctggaacgtgctgggcctgcaaggggcactgttgacccacttcctgcagcccatttatctcaaatctgtcacattgggttaccttttcagccaagggcatctgacccgtgctatttgctgtcgtgtgacaagagatgggagtgcatttgaggatggactacgacatccctttattgtcaaccaccccaaggttggcagagtcagcatatatgattccaaaaggcaatccgggaagactaaggagacaagcgtcaactggtgtctggctgatggctatgacctggagatcctggacggtaccagaggcactgtggatgggccacggaatgaattgtcccgggtctccaaaaagaacatttttcttctatttaagaagctctgctccttccgttaccgcagggatctactgagactctcctatggtgaggccaagaaagctgcccgtgactacgagacggccaagaactacttcaaaaaaggcctgaaggatatgggctatgggaactggattagcaaaccccaggaggaaaagaacttttatctctgcccagta

SEQ ID NO: 35 - RNA, 151 nt, synthetic construct
  source 1..151: mol_type = other RNA
catttctagtttggagatgacagtttccttagtaaccacagattgtgtcactagagtaacagtctgactggcagctagtgtttttgaaagaaagaagaaaagaaaaaaaaaagggaatatgagaaacacaagcaaaagagccaattttata

SEQ ID NO: 36 - RNA, 151 nt, synthetic construct
  source 1..151: mol_type = other RNA
acccaccatcactctctgtgattttataactcgatcaagcagagacagccagtctgtaagttctgtccaagctcggttgaagtctgccagtgcaggtacctccaacagcaaagaagatggcatttctagtttggagatgacagtttcctta

SEQ ID NO: 37 - RNA, 151 nt, synthetic construct
  source 1..151: mol_type = other RNA
gcagagacagccagtctgtaagttctgtccaagctcggttgaagtctgccagtgcaggtacctccaacagcaaagaagatggcatttctagtttggagatgacagtttccttagtaaccacagattgtgtcactagagtaacagtctgact

SEQ ID NO: 38 - RNA, 151 nt, synthetic construct
  source 1..151: mol_type = other RNA
cagtctgtaagttctgtccaagctcggttgaagtctgccagtgcaggtacctccaacagcaaagaagatggcatttctagtttggagatgacagtttccttagtaaccacagattgtgtcactagagtaacagtctgactggcagctagtg

SEQ ID NO: 39 - RNA, 71 nt, synthetic construct
  source 1..71: mol_type = other RNA
ttctagtttggagatgacagtttccttagtaaccacagattgtgtcactagagtaacagtctgactggcag

SEQ ID NO: 40 - RNA, 71 nt, synthetic construct
  source 1..71: mol_type = other RNA
caagcagagacagccagtctgtaagttctgtccaagctcggttgaagtctgccagtgcaggtacctccaac

SEQ ID NO: 41 - RNA, 31 nt, synthetic construct
  source 1..31: mol_type = other RNA
tccaagctcggttgaagtctgccagtgcagg

SEQ ID NO: 42 - RNA, 39 nt, synthetic construct
  source 1..39: mol_type = other RNA
gttctgtccaagctcggttgaagtctgccagtgcaggta

SEQ ID NO: 43 - RNA, 47 nt, synthetic construct
  source 1..47: mol_type = other RNA
gtctgtaagttctgtccaagctcggttgaagtctgccagtgcaggta

SEQ ID NO: 44 - RNA, 55 nt, synthetic construct
  source 1..55: mol_type = other RNA
gacagccagtctgtaagttctgtccaagctcggttgaagtctgccagtgcaggta

SEQ ID NO: 45 - RNA, 63 nt, synthetic construct
  source 1..63: mol_type = other RNA
gacagccagtctgtaagttctgtccaagctcggttgaagtctgccagtgcaggtacctccaac

SEQ ID NO: 46 - RNA, 111 nt, synthetic construct
  source 1..111: mol_type = other RNA
gaatctaaagtgcggggggacatcggcaggtcaatggtattgctgcaggtcgttccgtaggaaatggggggcagcaggaggggaaacggggggttgacaagacacaatgga